SEQUENCE LISTING (209 sequences)

SEQ ID NO: 1 - RNA, 21 nt, synthetic construct
  source 1..21: mol_type = other RNA
ttgcacctacgttcaattcca

SEQ ID NO: 2 - RNA, 21 nt, synthetic construct
  source 1..21: mol_type = other RNA
tttgaacgcgctctctcgctg

SEQ ID NO: 3 - RNA, 21 nt, synthetic construct
  source 1..21: mol_type = other RNA
ttgctcagatcgcaaacgttc

SEQ ID NO: 4 - RNA, 21 nt, synthetic construct
  source 1..21: mol_type = other RNA
ttgacatcaaatcctgccatc

SEQ ID NO: 5 - RNA, 21 nt, synthetic construct
  source 1..21: mol_type = other RNA
gtattgccaaaataagtgagt

SEQ ID NO: 6 - RNA, 21 nt, synthetic construct
  source 1..21: mol_type = other RNA
tactcttcttatgatacgtta

SEQ ID NO: 7 - RNA, 21 nt, synthetic construct
  source 1..21: mol_type = other RNA
tcgacaccgtaactgccgttc

SEQ ID NO: 8 - RNA, 21 nt, synthetic construct
  source 1..21: mol_type = other RNA
ttgaaaagagttgtgaattta

SEQ ID NO: 9 - RNA, 21 nt, synthetic construct
  source 1..21: mol_type = other RNA
ttaccggcgaaccaatctgta

SEQ ID NO: 10 - RNA, 21 nt, synthetic construct
  source 1..21: mol_type = other RNA
ttacgtttcttaattgctgta

SEQ ID NO: 11 - RNA, 21 nt, synthetic construct
  source 1..21: mol_type = other RNA
tcagatttttcaaggtaatct

SEQ ID NO: 12 - RNA, 21 nt, synthetic construct
  source 1..21: mol_type = other RNA
ttccgcccattcgttaccggc

SEQ ID NO: 13 - RNA, 21 nt, synthetic construct
  source 1..21: mol_type = other RNA
acatggcggcatgaccctgtc

SEQ ID NO: 14 - RNA, 21 nt, synthetic construct
  source 1..21: mol_type = other RNA
tcagatttttcaaggtaatct

SEQ ID NO: 15 - RNA, 21 nt, synthetic construct
  source 1..21: mol_type = other RNA
agagtaagaacttcgaacaaa

SEQ ID NO: 16 - RNA, 21 nt, synthetic construct
  source 1..21: mol_type = other RNA
atagtctgcactttcgacgaa

SEQ ID NO: 17 - RNA, 21 nt, synthetic construct
  source 1..21: mol_type = other RNA
aaatcagatttagtgtaaata

SEQ ID NO: 18 - RNA, 21 nt, synthetic construct
  source 1..21: mol_type = other RNA
actcagctcccgccacagaaa

SEQ ID NO: 19 - RNA, 21 nt, synthetic construct
  source 1..21: mol_type = other RNA
atggacaacccgttcaccaca

SEQ ID NO: 20 - RNA, 21 nt, synthetic construct
  source 1..21: mol_type = other RNA
aaacctaggagatggtttcaa

SEQ ID NO: 21 - RNA, 21 nt, synthetic construct
  source 1..21: mol_type = other RNA
aatgaagaatctctcagccac

SEQ ID NO: 22 - RNA, 21 nt, synthetic construct
  source 1..21: mol_type = other RNA
aaagcgacaaggagctcatca

SEQ ID NO: 23 - RNA, 21 nt, synthetic construct
  source 1..21: mol_type = other RNA
aatagccgcgaccaggtcttc

SEQ ID NO: 24 - RNA, 21 nt, synthetic construct
  source 1..21: mol_type = other RNA
ataaaagaacatttattaaac

SEQ ID NO: 25 - RNA, 21 nt, synthetic construct
  source 1..21: mol_type = other RNA
atacgcatgggtttgaccatc

SEQ ID NO: 26 - RNA, 21 nt, synthetic construct
  source 1..21: mol_type = other RNA
aaagcaccttccgcccattcg

SEQ ID NO: 27 - RNA, 21 nt, synthetic construct
  source 1..21: mol_type = other RNA
gaattgaacgtaggtgcaatg

SEQ ID NO: 28 - RNA, 21 nt, synthetic construct
  source 1..21: mol_type = other RNA
gcgagagagcgcgttcaaatc

SEQ ID NO: 29 - RNA, 21 nt, synthetic construct
  source 1..21: mol_type = other RNA
acgtttgcgatctgagcaacg

SEQ ID NO: 30 - RNA, 21 nt, synthetic construct
  source 1..21: mol_type = other RNA
tggcaggatttgatgtcaaga

SEQ ID NO: 31 - RNA, 21 nt, synthetic construct
  source 1..21: mol_type = other RNA
tcacttattttggcaatacca

SEQ ID NO: 32 - RNA, 21 nt, synthetic construct
  source 1..21: mol_type = other RNA
acgtatcataagaagagtata

SEQ ID NO: 33 - RNA, 21 nt, synthetic construct
  source 1..21: mol_type = other RNA
acggcagttacggtgtcgaca

SEQ ID NO: 34 - RNA, 21 nt, synthetic construct
  source 1..21: mol_type = other RNA
aattcacaactcttttcaaca

SEQ ID NO: 35 - RNA, 21 nt, synthetic construct
  source 1..21: mol_type = other RNA
cagattggttcgccggtaacg

SEQ ID NO: 36 - RNA, 21 nt, synthetic construct
  source 1..21: mol_type = other RNA
cagcaattaagaaacgtaatg

SEQ ID NO: 37 - RNA, 21 nt, synthetic construct
  source 1..21: mol_type = other RNA
attaccttgaaaaatctgatg

SEQ ID NO: 38 - RNA, 21 nt, synthetic construct
  source 1..21: mol_type = other RNA
cggtaacgaatgggcggaagg

SEQ ID NO: 39 - RNA, 21 nt, synthetic construct
  source 1..21: mol_type = other RNA
cagggtcatgccgccatgtga

SEQ ID NO: 40 - RNA, 21 nt, synthetic construct
  source 1..21: mol_type = other RNA
catgttagacttcgacagtct

SEQ ID NO: 41 - RNA, 21 nt, synthetic construct
  source 1..21: mol_type = other RNA
tgttcgaagttcttactctct

SEQ ID NO: 42 - RNA, 21 nt, synthetic construct
  source 1..21: mol_type = other RNA
cgtcgaaagtgcagactattc

SEQ ID NO: 43 - RNA, 21 nt, synthetic construct
  source 1..21: mol_type = other RNA
tttacactaaatctgatttct

SEQ ID NO: 44 - RNA, 21 nt, synthetic construct
  source 1..21: mol_type = other RNA
tctgtggcgggagctgagttg

SEQ ID NO: 45 - RNA, 21 nt, synthetic construct
  source 1..21: mol_type = other RNA
tggtgaacgggttgtccatcc

SEQ ID NO: 46 - RNA, 21 nt, synthetic construct
  source 1..21: mol_type = other RNA
gaaaccatctcctaggtttct

SEQ ID NO: 47 - RNA, 21 nt, synthetic construct
  source 1..21: mol_type = other RNA
ggctgagagattcttcatttc

SEQ ID NO: 48 - RNA, 21 nt, synthetic construct
  source 1..21: mol_type = other RNA
atgagctccttgtcgcttttg

SEQ ID NO: 49 - RNA, 21 nt, synthetic construct
  source 1..21: mol_type = other RNA
agacctggtcgcggctattta

SEQ ID NO: 50 - RNA, 21 nt, synthetic construct
  source 1..21: mol_type = other RNA
ttaataaatgttcttttattt

SEQ ID NO: 51 - RNA, 21 nt, synthetic construct
  source 1..21: mol_type = other RNA
tggtcaaacccatgcgtatcg

SEQ ID NO: 52 - RNA, 21 nt, synthetic construct
  source 1..21: mol_type = other RNA
aatgggcggaaggtgctttct

SEQ ID NO: 53 - RNA, 21 nt, synthetic construct
  source 1..21: mol_type = other RNA
acacacacacgcacacacaca

SEQ ID NO: 54 - RNA, 21 nt, synthetic construct
  source 1..21: mol_type = other RNA
ttaaagtcctactggtacctt

SEQ ID NO: 55 - RNA, 21 nt, synthetic construct
  source 1..21: mol_type = other RNA
tcacactcagtagccattttc

SEQ ID NO: 56 - RNA, 21 nt, synthetic construct
  source 1..21: mol_type = other RNA
attcaccaacatcatatccag

SEQ ID NO: 57 - RNA, 21 nt, synthetic construct
  source 1..21: mol_type = other RNA
ataactccttgtcgcccagat

SEQ ID NO: 58 - RNA, 21 nt, synthetic construct
  source 1..21: mol_type = other RNA
attgcccatctatgggagata

SEQ ID NO: 59 - RNA, 21 nt, synthetic construct
  source 1..21: mol_type = other RNA
taacgaaacgcattgcccatc

SEQ ID NO: 60 - RNA, 21 nt, synthetic construct
  source 1..21: mol_type = other RNA
aacaccgcttacgattcccaa

SEQ ID NO: 61 - RNA, 21 nt, synthetic construct
  source 1..21: mol_type = other RNA
aaacaccgcttacgattccca

SEQ ID NO: 62 - RNA, 21 nt, synthetic construct
  source 1..21: mol_type = other RNA
aaggcagtactagagtcttcc

SEQ ID NO: 63 - RNA, 21 nt, synthetic construct
  source 1..21: mol_type = other RNA
ttatatactaatacgcaccaa

SEQ ID NO: 64 - RNA, 21 nt, synthetic construct
  source 1..21: mol_type = other RNA
tacttatatactaatacgcac

SEQ ID NO: 65 - RNA, 21 nt, synthetic construct
  source 1..21: mol_type = other RNA
atagatatagtattatgtaca

SEQ ID NO: 66 - RNA, 21 nt, synthetic construct
  source 1..21: mol_type = other RNA
ttactaaaaccagatgtgttc

SEQ ID NO: 67 - RNA, 21 nt, synthetic construct
  source 1..21: mol_type = other RNA
aagaggaattgaacctcaaaa

SEQ ID NO: 68 - RNA, 21 nt, synthetic construct
  source 1..21: mol_type = other RNA
aacgtcttattaagtcgcgaa

SEQ ID NO: 69 - RNA, 21 nt, synthetic construct
  source 1..21: mol_type = other RNA
aacaagcttcttatcatattc

SEQ ID NO: 70 - RNA, 21 nt, synthetic construct
  source 1..21: mol_type = other RNA
ttctccacgactgaccatttt

SEQ ID NO: 71 - RNA, 21 nt, synthetic construct
  source 1..21: mol_type = other RNA
aaggcgcctcagagatttgta

SEQ ID NO: 72 - RNA, 21 nt, synthetic construct
  source 1..21: mol_type = other RNA
taccctgaaactagcacgttg

SEQ ID NO: 73 - RNA, 21 nt, synthetic construct
  source 1..21: mol_type = other RNA
tgtgtgtgcgtgtgtgtgtgt

SEQ ID NO: 74 - RNA, 21 nt, synthetic construct
  source 1..21: mol_type = other RNA
ggtaccagtaggactttaact

SEQ ID NO: 75 - RNA, 21 nt, synthetic construct
  source 1..21: mol_type = other RNA
aaatggctactgagtgtgacc

SEQ ID NO: 76 - RNA, 21 nt, synthetic construct
  source 1..21: mol_type = other RNA
ggatatgatgttggtgaatta

SEQ ID NO: 77 - RNA, 21 nt, synthetic construct
  source 1..21: mol_type = other RNA
ctgggcgacaaggagttatct

SEQ ID NO: 78 - RNA, 21 nt, synthetic construct
  source 1..21: mol_type = other RNA
tctcccatagatgggcaatgc

SEQ ID NO: 79 - RNA, 21 nt, synthetic construct
  source 1..21: mol_type = other RNA
tgggcaatgcgtttcgttaca

SEQ ID NO: 80 - RNA, 21 nt, synthetic construct
  source 1..21: mol_type = other RNA
gggaatcgtaagcggtgtttt

SEQ ID NO: 81 - RNA, 21 nt, synthetic construct
  source 1..21: mol_type = other RNA
ggaatcgtaagcggtgttttg

SEQ ID NO: 82 - RNA, 21 nt, synthetic construct
  source 1..21: mol_type = other RNA
aagactctagtactgcctttc

SEQ ID NO: 83 - RNA, 21 nt, synthetic construct
  source 1..21: mol_type = other RNA
ggtgcgtattagtatataagt

SEQ ID NO: 84 - RNA, 21 nt, synthetic construct
  source 1..21: mol_type = other RNA
gcgtattagtatataagtatt

SEQ ID NO: 85 - RNA, 21 nt, synthetic construct
  source 1..21: mol_type = other RNA
tacataatactatatctatag

SEQ ID NO: 86 - RNA, 21 nt, synthetic construct
  source 1..21: mol_type = other RNA
acacatctggttttagtaagc

SEQ ID NO: 87 - RNA, 21 nt, synthetic construct
  source 1..21: mol_type = other RNA
ttgaggttcaattcctcttac

SEQ ID NO: 88 - RNA, 21 nt, synthetic construct
  source 1..21: mol_type = other RNA
cgcgacttaataagacgttag

SEQ ID NO: 89 - RNA, 21 nt, synthetic construct
  source 1..21: mol_type = other RNA
atatgataagaagcttgtttc

SEQ ID NO: 90 - RNA, 21 nt, synthetic construct
  source 1..21: mol_type = other RNA
aatggtcagtcgtggagaaat

SEQ ID NO: 91 - RNA, 21 nt, synthetic construct
  source 1..21: mol_type = other RNA
caaatctctgaggcgcctttg

SEQ ID NO: 92 - RNA, 21 nt, synthetic construct
  source 1..21: mol_type = other RNA
acgtgctagtttcagggtacg

SEQ ID NO: 93 - RNA, 21 nt, synthetic construct
  source 1..21: mol_type = other RNA
aaagagcagttttgtcaacaa

SEQ ID NO: 94 - RNA, 21 nt, synthetic construct
  source 1..21: mol_type = other RNA
aagcttctctttcccgaattt

SEQ ID NO: 95 - RNA, 21 nt, synthetic construct
  source 1..21: mol_type = other RNA
aaaagcgtttatcgtcgatgg

SEQ ID NO: 96 - RNA, 21 nt, synthetic construct
  source 1..21: mol_type = other RNA
aagctgaggatagcgcttgta

SEQ ID NO: 97 - RNA, 21 nt, synthetic construct
  source 1..21: mol_type = other RNA
atatttctcttcttggccttc

SEQ ID NO: 98 - RNA, 21 nt, synthetic construct
  source 1..21: mol_type = other RNA
atgggccgatggagtggagca

SEQ ID NO: 99 - RNA, 21 nt, synthetic construct
  source 1..21: mol_type = other RNA
atttcgaaaatgtccacgtct

SEQ ID NO: 100 - RNA, 21 nt, synthetic construct
  source 1..21: mol_type = other RNA
aatccttttgacccattccaa

SEQ ID NO: 101 - RNA, 21 nt, synthetic construct
  source 1..21: mol_type = other RNA
aaagtcaagatacctcttttg

SEQ ID NO: 102 - RNA, 21 nt, synthetic construct
  source 1..21: mol_type = other RNA
atgttcttcaggagcaacacg

SEQ ID NO: 103 - RNA, 21 nt, synthetic construct
  source 1..21: mol_type = other RNA
agaacggcttggatggcaaca

SEQ ID NO: 104 - RNA, 21 nt, synthetic construct
  source 1..21: mol_type = other RNA
aagcatacaaagaaagaacgg

SEQ ID NO: 105 - RNA, 21 nt, synthetic construct
  source 1..21: mol_type = other RNA
agaagcatacaaagaaagaac

SEQ ID NO: 106 - RNA, 21 nt, synthetic construct
  source 1..21: mol_type = other RNA
aagcaacatagcaaagcttct

SEQ ID NO: 107 - RNA, 21 nt, synthetic construct
  source 1..21: mol_type = other RNA
gttgacaaaactgctcttttc

SEQ ID NO: 108 - RNA, 21 nt, synthetic construct
  source 1..21: mol_type = other RNA
attcgggaaagagaagcttca

SEQ ID NO: 109 - RNA, 21 nt, synthetic construct
  source 1..21: mol_type = other RNA
atcgacgataaacgcttttga

SEQ ID NO: 110 - RNA, 21 nt, synthetic construct
  source 1..21: mol_type = other RNA
caagcgctatcctcagctttg

SEQ ID NO: 111 - RNA, 21 nt, synthetic construct
  source 1..21: mol_type = other RNA
aggccaagaagagaaatatcg

SEQ ID NO: 112 - RNA, 21 nt, synthetic construct
  source 1..21: mol_type = other RNA
ctccactccatcggcccatag

SEQ ID NO: 113 - RNA, 21 nt, synthetic construct
  source 1..21: mol_type = other RNA
acgtggacattttcgaaatcg

SEQ ID NO: 114 - RNA, 21 nt, synthetic construct
  source 1..21: mol_type = other RNA
ggaatgggtcaaaaggattcg

SEQ ID NO: 115 - RNA, 21 nt, synthetic construct
  source 1..21: mol_type = other RNA
aaagaggtatcttgactttga

SEQ ID NO: 116 - RNA, 21 nt, synthetic construct
  source 1..21: mol_type = other RNA
tgttgctcctgaagaacatcc

SEQ ID NO: 117 - RNA, 21 nt, synthetic construct
  source 1..21: mol_type = other RNA
ttgccatccaagccgttcttt

SEQ ID NO: 118 - RNA, 21 nt, synthetic construct
  source 1..21: mol_type = other RNA
gttctttctttgtatgcttct

SEQ ID NO: 119 - RNA, 21 nt, synthetic construct
  source 1..21: mol_type = other RNA
tctttctttgtatgcttctgg

SEQ ID NO: 120 - RNA, 21 nt, synthetic construct
  source 1..21: mol_type = other RNA
aagctttgctatgttgctttg

SEQ ID NO: 121 - RNA, 21 nt, synthetic construct
  source 1..21: mol_type = other RNA
ccctccaactcgcgctccatt

SEQ ID NO: 122 - RNA, 21 nt, synthetic construct
  source 1..21: mol_type = other RNA
taacacttcctcttcgcaatc

SEQ ID NO: 123 - RNA, 21 nt, synthetic construct
  source 1..21: mol_type = other RNA
taagggaagcgagtgagactt

SEQ ID NO: 124 - RNA, 21 nt, synthetic construct
  source 1..21: mol_type = other RNA
ttgtcgcaagtgaattcgagg

SEQ ID NO: 125 - RNA, 21 nt, synthetic construct
  source 1..21: mol_type = other RNA
ttgcttgcatccgggattgtc

SEQ ID NO: 126 - RNA, 21 nt, synthetic construct
  source 1..21: mol_type = other RNA
tatctcttgcttgcatccggg

SEQ ID NO: 127 - RNA, 21 nt, synthetic construct
  source 1..21: mol_type = other RNA
ctcatcaaagactgtagaaag

SEQ ID NO: 128 - RNA, 21 nt, synthetic construct
  source 1..21: mol_type = other RNA
tatcctcatcaaagactgtag

SEQ ID NO: 129 - RNA, 21 nt, synthetic construct
  source 1..21: mol_type = other RNA
tttgacgataccatacatgtt

SEQ ID NO: 130 - RNA, 21 nt, synthetic construct
  source 1..21: mol_type = other RNA
tttcacttaattctaccccag

SEQ ID NO: 131 - RNA, 21 nt, synthetic construct
  source 1..21: mol_type = other RNA
tttttccctttcgaagcaaac

SEQ ID NO: 132 - RNA, 21 nt, synthetic construct
  source 1..21: mol_type = other RNA
tatcaggaaggagcttttgga

SEQ ID NO: 133 - RNA, 21 nt, synthetic construct
  source 1..21: mol_type = other RNA
tatttgtgcgacttgctctct

SEQ ID NO: 134 - RNA, 21 nt, synthetic construct
  source 1..21: mol_type = other RNA
taataacatatataggtttcc

SEQ ID NO: 135 - RNA, 21 nt, synthetic construct
  source 1..21: mol_type = other RNA
tttcgtactatcaatcgagat

SEQ ID NO: 136 - RNA, 21 nt, synthetic construct
  source 1..21: mol_type = other RNA
taaccccgctttcgtactatc

SEQ ID NO: 137 - RNA, 21 nt, synthetic construct
  source 1..21: mol_type = other RNA
ttactcttcaataaccccgct

SEQ ID NO: 138 - RNA, 21 nt, synthetic construct
  source 1..21: mol_type = other RNA
ttcttttgtgtgcttctactg

SEQ ID NO: 139 - RNA, 21 nt, synthetic construct
  source 1..21: mol_type = other RNA
tatccccccttcttcgagatc

SEQ ID NO: 140 - RNA, 21 nt, synthetic construct
  source 1..21: mol_type = other RNA
tatcagattttccttcagtga

SEQ ID NO: 141 - RNA, 21 nt, synthetic construct
  source 1..21: mol_type = other RNA
tttcgctcgatgtgaagttgc

SEQ ID NO: 142 - RNA, 21 nt, synthetic construct
  source 1..21: mol_type = other RNA
tttccctgagacttagctttc

SEQ ID NO: 143 - RNA, 21 nt, synthetic construct
  source 1..21: mol_type = other RNA
tatcctgccatctatatacat

SEQ ID NO: 144 - RNA, 21 nt, synthetic construct
  source 1..21: mol_type = other RNA
ttcaaccccaagaaaatgaac

SEQ ID NO: 145 - RNA, 21 nt, synthetic construct
  source 1..21: mol_type = other RNA
tatacacttttcaagtcgcac

SEQ ID NO: 146 - RNA, 21 nt, synthetic construct
  source 1..21: mol_type = other RNA
tatcggtgagagcatatacac

SEQ ID NO: 147 - RNA, 21 nt, synthetic construct
  source 1..21: mol_type = other RNA
taacatctgcggaataagtgg

SEQ ID NO: 148 - RNA, 21 nt, synthetic construct
  source 1..21: mol_type = other RNA
tatccctgcattcaagactac

SEQ ID NO: 149 - RNA, 21 nt, synthetic construct
  source 1..21: mol_type = other RNA
ttgcgcagcttcatcgtcgga

SEQ ID NO: 150 - RNA, 21 nt, synthetic construct
  source 1..21: mol_type = other RNA
ttcctcagctgatcgagggta

SEQ ID NO: 151 - RNA, 21 nt, synthetic construct
  source 1..21: mol_type = other RNA
tggagcgcgagttggagggat

SEQ ID NO: 152 - RNA, 21 nt, synthetic construct
  source 1..21: mol_type = other RNA
ttgcgaagaggaagtgttagc

SEQ ID NO: 153 - RNA, 21 nt, synthetic construct
  source 1..21: mol_type = other RNA
gtctcactcgcttcccttacc

SEQ ID NO: 154 - RNA, 21 nt, synthetic construct
  source 1..21: mol_type = other RNA
tcgaattcacttgcgacaatc

SEQ ID NO: 155 - RNA, 21 nt, synthetic construct
  source 1..21: mol_type = other RNA
caatcccggatgcaagcaaga

SEQ ID NO: 156 - RNA, 21 nt, synthetic construct
  source 1..21: mol_type = other RNA
cggatgcaagcaagagatacc

SEQ ID NO: 157 - RNA, 21 nt, synthetic construct
  source 1..21: mol_type = other RNA
ttctacagtctttgatgagga

SEQ ID NO: 158 - RNA, 21 nt, synthetic construct
  source 1..21: mol_type = other RNA
acagtctttgatgaggatatg

SEQ ID NO: 159 - RNA, 21 nt, synthetic construct
  source 1..21: mol_type = other RNA
catgtatggtatcgtcaaatt

SEQ ID NO: 160 - RNA, 21 nt, synthetic construct
  source 1..21: mol_type = other RNA
ggggtagaattaagtgaaacg

SEQ ID NO: 161 - RNA, 21 nt, synthetic construct
  source 1..21: mol_type = other RNA
ttgcttcgaaagggaaaaaca

SEQ ID NO: 162 - RNA, 21 nt, synthetic construct
  source 1..21: mol_type = other RNA
caaaagctccttcctgatatc

SEQ ID NO: 163 - RNA, 21 nt, synthetic construct
  source 1..21: mol_type = other RNA
agagcaagtcgcacaaataca

SEQ ID NO: 164 - RNA, 21 nt, synthetic construct
  source 1..21: mol_type = other RNA
aaacctatatatgttattact

SEQ ID NO: 165 - RNA, 21 nt, synthetic construct
  source 1..21: mol_type = other RNA
ctcgattgatagtacgaaagc

SEQ ID NO: 166 - RNA, 21 nt, synthetic construct
  source 1..21: mol_type = other RNA
tagtacgaaagcggggttatt

SEQ ID NO: 167 - RNA, 21 nt, synthetic construct
  source 1..21: mol_type = other RNA
cggggttattgaagagtaata

SEQ ID NO: 168 - RNA, 21 nt, synthetic construct
  source 1..21: mol_type = other RNA
gtagaagcacacaaaagaacg

SEQ ID NO: 169 - RNA, 21 nt, synthetic construct
  source 1..21: mol_type = other RNA
tctcgaagaaggggggatatg

SEQ ID NO: 170 - RNA, 21 nt, synthetic construct
  source 1..21: mol_type = other RNA
actgaaggaaaatctgatatc

SEQ ID NO: 171 - RNA, 21 nt, synthetic construct
  source 1..21: mol_type = other RNA
aacttcacatcgagcgaaagc

SEQ ID NO: 172 - RNA, 21 nt, synthetic construct
  source 1..21: mol_type = other RNA
aagctaagtctcagggaaatg

SEQ ID NO: 173 - RNA, 21 nt, synthetic construct
  source 1..21: mol_type = other RNA
gtatatagatggcaggatacg

SEQ ID NO: 174 - RNA, 21 nt, synthetic construct
  source 1..21: mol_type = other RNA
tcattttcttggggttgaagt

SEQ ID NO: 175 - RNA, 21 nt, synthetic construct
  source 1..21: mol_type = other RNA
gcgacttgaaaagtgtatatg

SEQ ID NO: 176 - RNA, 21 nt, synthetic construct
  source 1..21: mol_type = other RNA
gtatatgctctcaccgatagg

SEQ ID NO: 177 - RNA, 21 nt, synthetic construct
  source 1..21: mol_type = other RNA
acttattccgcagatgttaaa

SEQ ID NO: 178 - RNA, 21 nt, synthetic construct
  source 1..21: mol_type = other RNA
agtcttgaatgcagggatagg

SEQ ID NO: 179 - RNA, 21 nt, synthetic construct
  source 1..21: mol_type = other RNA
cgacgatgaagctgcgcaacc

SEQ ID NO: 180 - RNA, 21 nt, synthetic construct
  source 1..21: mol_type = other RNA
ccctcgatcagctgaggaaag

SEQ ID NO: 181 - RNA, 170 nt, synthetic construct
  source 1..170: mol_type = other RNA
gggcgaattgggtaccgggcattacgtttcttaattgctgtatactcttcttatgatacgttatcagatttttcaaggtaatcttgatgagctccttgtcgcttttttgttcgaagttcttactctttcgtcgaaagtgcagactattctcagatctgccggtctcccta

SEQ ID NO: 182 - RNA, 178 nt, synthetic construct
  source 1..178: mol_type = other RNA
gggcgaattgggtaccgggccattacgtttcttaattgctgtaatactcttcttatgatacgttattcagatttttcaaggtaatcttatgatgagctccttgtcgctttatttgttcgaagttcttactctgttcgtcgaaagtgcagactattcatcagatctgccggtctcccta

SEQ ID NO: 183 - RNA, 170 nt, synthetic construct
  source 1..170: mol_type = other RNA
gggcgaattgggtaccggggaataaccgggtacatcgcgagacgagatatctatctgagcgtcgtcggcttcacactcttcactgtagtagaaatcagatttagtgtaaatagccgcgaccaggtcttcaaaacacttcatggttcggttcagatctgccggtctcccta

SEQ ID NO: 184 - RNA, 170 nt, synthetic construct
  source 1..170: mol_type = other RNA
gggcgaattgggtaccgggcaagatctgagtccggacttgtatagttcatccatgccatgtgtaatcccagcagctgttacaaactcaagaaggaccatgtggtctctcttttcgttgggatctttcgaaagggcagattgtgtggacatcagatctgccggtctcccta

SEQ ID NO: 185 - RNA, 170 nt, synthetic construct
  source 1..170: mol_type = other RNA
atcccgcttaacccatggcccgtaatgcaaagaattaacgacatatgagaagaatactatgcaatagtctaaaaagttccattagaactactcgaggaacagcgaaaaaacaagcttcaagaatgagaaagcagctttcacgtctgataagagtctagacggccagaggg

SEQ ID NO: 186 - RNA, 178 nt, synthetic construct
  source 1..178: mol_type = other RNA
atcccgcttaacccatggcccggtaatgcaaagaattaacgacattatgagaagaatactatgcaataagtctaaaaagttccattagaatactactcgaggaacagcgaaataaacaagcttcaagaatgagacaagcagctttcacgtctgataagtagtctagacggccagaggg

SEQ ID NO: 187 - RNA, 170 nt, synthetic construct
  source 1..170: mol_type = other RNA
atcccgcttaacccatggccccttattggcccatgtagcgctctgctctatagatagactcgcagcagccgaagtgtgagaagtgacatcatctttagtctaaatcacatttatcggcgctggtccagaagttttgtgaagtaccaagccaagtctagacggccagaggg

SEQ ID NO: 188 - RNA, 170 nt, synthetic construct
  source 1..170: mol_type = other RNA
atcccgcttaacccatggcccgttctagactcaggcctgaacatatcaagtaggtacggtacacattagggtcgtcgacaatgtttgagttcttcctggtacaccagagagaaaagcaaccctagaaagctttcccgtctaacacacctgtagtctagacggccagaggg

SEQ ID NO: 189 - RNA, 3050 nt, Cucumber mosaic virus
  source 1..3050: mol_type = transcribed RNA
gtttatttacaagagcgtacggttcaacccctgcctcccctgtaaaactccctagacttaaatcttttctttctagtatcttttctatggctttccctgcccccgcattctcactagccaatcttttgaacggcagttacggtgtcgacactcccgaggatgtggaacgtttgcgatctgagcaacgcgaagaggctgctgcggcctgtcgtaattacaggcccctacccgctgtggatgtcagcgagagtgtcacagaggacgcgcattccctccgaactcctgacggagctcccgctgaagcggtgtctgatgagtttgtaacttatggtgctgaagattaccttgaaaaatctgatgatgagctccttgtcgcttttgagacgatggtcaaacccatgcgtatcggacaactatggtgccctgcgtttaataaatgttcttttatttccagcattgctatggccagagctttgttgttggcacctagaacatcccaccgaaccatgaagtgttttgaagacctggtcgcggctatttacactaaatctgatttctactacagtgaagagtgtgaagccgacgacgctcagatagatatctcgtctcgcgatgtacccggttattctttcgaaccgtggtcccgaacgtctggatttgaaccgccgcccatttgtgaagcgtgcgacatgatcatgtaccagtgcccgtgttttgattttaatgctttaaagaaatcgtgcgctgagaggaccttcgctgatgattatgttattgaaggtttagatggtgttgttgataatgcgactctgttgtcgaatttgggtccatttttggtacccgtgaagtgtcaatatgaaaaatgtccaacgccaaccatcgcgattcctccggatttaaaccgtgctactgatcgtgttgatatcaatttagttcaatccatttgtgactcgactctgcccactcatagtaattacgacgactcttttcatcaagtgttcgtcgaaagtgcagactattctatagatctggatcatgttagacttcgacagtctgatcttattgcaaaaattccagattcagggcatatgataccggttctgaacaccgggagcggtcacaagagagtaggtacaacgaaggaggtccttacagcaattaagaaacgtaatgctgacgttccagagctaggtgattccgttaatttgtctagattgagtaaagctgtggctgagagattcttcatttcatacatcaatggtaactctctagcatccagtaactttgtcaatgtcgttagtaacttccacgattacatggaaaaatggaagtcctcaggtctttcttatgatgatcttccggatcttcatgctgagaatttgcagttttatgaccacatgataaaatccgatgtgaaacctgtggtgagcgacacactcaatatcgacagaccggttccagctactataacgtatcataagaagagtataacctcccagttctcaccgttattcacagcgctattcgagcgcttccagagatgccttcgagaacgtattattcttcctgttggtaagatttcatcccttgagatggcaggatttgatgtcaagaacaagcactgcctcgagattgacctgtctaagtttgataagtctcaaggtgaatttcacttgctaatccaggaacacattttgaatggtctaggatgtccagctccgataactaagtggtggtgtgatttccatcgattctcttacattagagaccgtagagctggtgttggtatgcctattagtttccagagacgaactggcgatgcactcacttattttggcaataccatcgtcaccatggctgagtttgcctggtgttatgacaccgaccaattcgaaaagcttttattctcaggcgatgattctctaggattttcactgcttccccctgttggtgacccgagtaaattcacaactcttttcaacatggaagctaaggtgatggaacctgccgtaccatatatttgttcgaagttcttactctctgacgagttcggtaacacattttccgttccagatccattgcgcgaggttcagcggttaggaacaaagaaaattccctattctgacaatgatgaattcttgtttgctcacttcatgagctttgttgatcgattgaagtttttggaccgaatgtctcagtcgtgtatcgatcaactttcgattttcttcgaattgaaatacaagaagtctggggaagaggctgctttaatgttaggcgcctttaagaagtataccgctaatttccagtcctacaaagaactctattattcagatcgtcgtcagtgcgaattgatcaattcgttttgtagtacagagttcagggttgagcgtgtaaattccaacaaacagcgaaagaattatggaattgaacgtaggtgcaatgacaaacgtcgaactccaactggctcgtatggtggaggcgaagaagcagagacgaaggtctcacaaacagaatcgacgggaacgaggtcacaaaagtcccagcgagagagcgcgttcaaatctcagactattccgcttcctaccgttctatcaagtggatggttcggaactgacagggtcatgccgccatgtgaacgtggcggagttacccgagtctgaggcctctcgtttagagttatcggcggaagaccatgattttgacgatacagattggttcgccggtaacgaatgggcggaaggtgctttctgaaacctccccttccgcatctccctccggttttctgtggcgggagctgagttggcagtattgctataaactgtctgaagtcactaaacacattgtggtgaacgggttgtccatccagcttacggctaaaatggtcagtcgtagaggaatctacgccagcagacttacaagtctctgaggcacctttgaaaccatctcctaggtttcttcggaaggacttcggtccgtgtacttctagcacaacgtgctagtttcagggtacgggtgccccccacttttgtggggcctccaaaaggagacca

SEQ ID NO: 190 - RNA, 2216 nt, Cucumber mosaic virus
  source 1..2216: mol_type = transcribed RNA
gtaatcttaccactgtgtgtgtgcgtgtgtgtgtgtcgagtcgtgttgtccgcacatttgagtcgtgctgtccgcacatattttatcttttgggtacagtgtgttagatttcccgaggcatggctttccaaggtaccagtaggactttaactcaacagtcctcagcggctacgtctgacgatcttcaaaagatattatttagccctgaagccattaagaaaatggctactgagtgtgacctaggccggcatcattggatgcgcgctgataatgctatttcagtccggcccctcgttcccgaagtaacccacggtcgtattgcttccttctttaagtctggatatgatgttggtgaattatgctcaaaaggatacatgagtgtccctcaagtgttatgtgctgttactcgaacagtttccactgatgctgaagggtctttgagaatttacttagctgatctgggcgacaaggagttatctcccatagatgggcaatgcgtttcgttacataaccatgatcttcccgctttggtgtctttccaaccgacgtatgattgtcctatggaaacagttgggaatcgtaagcggtgttttgctgtcgttatcgaaagacatggttacattgggtataccggtaccacagctagcgtgtgtagtaattggcaagcaaggttttcatccaagaataacaactacactcatatcgcagctgggaagactctagtactgcctttcaacagattagctgagcaaacaaaaccgtcagctgtcgctcgcctgttgaagtcgcaattgaacaacattgaatcttcgcaatatttgttaacgaatgcgaagattaatcaaaatgcgcgcagtgagtccgaggatttaaatgttgagagccctcccgccgcaatcgggagttcttccgcgtcccgctccgaagccttcagaccgcaggtggttaacggtctttagcactttggtgcgtattagtatataagtatttgtgagtctgtacataatactatatctatagtgtcctgtgtgagttgatacagtagacatctgtgacgcgatgccgtgttgagaagggaacacatctggttttagtaagcctacatcatagttttgaggttcaattcctcttactccctgttgagccccttactttctcatggatgcttctccgcgagattgcgttattgtctactgactatatagagagtgtttgtgctgtgttttctcttttgtgtcgtagaattgagtcgagtcatggacaaatctgaatcaaccagtgctggtcgtaaccgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtagacctgggtacacgttcacatctattaccctaaagccaccaaaaatagaccgtgggtcttattacggtaaaaggttgttactacctgattcagtcacggaatatgataagaagcttgtttcgcgcattcaaattcgagttaatcctttgccgaaatttgattctaccgtgtgggtgacagtccgtaaagttcctgcctcctcggacttatccgttgccgccatctctgctatgttcgcggacggagcctcaccggtactggtttatcagtatgccgcatctggagtccaagccaacaacaaactgttgtatgatctttcggcgatgcgcgctgatataggtgacatgagaaagtacgccgtcctcgtgtattcaaaagacgatgcgctcgagacggacgagctagtacttcatgttgacatcgagcaccaacgcattcccacatctggagtgctcccagtctgattccgtgttcccagaatcctccctccgatctctgtggcgggacgtgagttggcagttctgctataaactgtctgaagtcactaaacgttttttacggtgaacgggttgtccatccagcttacggctaaaatggtcagtcgtggagaaatccacgccagcagatttacaaatctctgaggcgcctttgaaaccatctcctaggtttcttcggaaggacttcggtccgtgtacctctagcacaacgtgctagtttcagggtacgggtgcccccccactttcgtgggggcctccaaaaggagacca

SEQ ID NO: 191 - RNA, 2259 nt, Meloidogyne incognita
  source 1..2259: mol_type = transcribed RNA
atgcctccaatagaaattttggtgcaggctccacaacagcctataaaggtttctggacgtgaggaagattcaatgaataaagaaccctcaatgagtggtcaacaaattgttggtttaattattccacctcctgatattcgaacaattgttgacaaaactgctcttttcgttgcacgtaatggtgttgaatttgaaaataaaattcgggaaagagaagcttcaaatatacgttttaattttcttacacctgtcgacccgtaccatgcttattacaaaaataaagtgaaggaattcgagactggagttgcttctgctgaattattaactaaacttaaaattcctgatgctgttcgtgaacatatacaaaaggctgaatttgttccaaaaaatccgccaaaaccttttgaattttgtgctgatccatcgacgataaacgcttttgatttagatcttatccaacttaccgctctatttgttgcaagaaatggacgtcaatttctcacacaattaatgaatcgagaggtccgcaattttcaattcgactttttaaaacctcaacattccaattttcaatattttacaaaattagtggaacaatacactaaggtgataattccacccaggaatgtgtacgatgaactacgcaattctgcttcacacaaaaaaattttggaagacgttcggtatcgtgttggttgggaaaaatttcaacgtgcagttaaggacagagaagatgctgaagttgaacaagaaaggatggcttataatcaaattgattggcatgattttgtagttgttcaaactgttgattttcaaccttctgaaaccttgaatttaccttcactttgtactcctcgagatgttggagctcgaattttacttcaacaacgaactgaagcagctaaagctgcagctgaaagtgttgcaatggatatggatgaatcagattctgaagatgaagaagagaaaaagaaacagaaaaatagggaagattttgatgatatacaacatcaagtcgatgggatggatattactgaaaaacaacatgctggctctactgttactcagccaacaccagctgcacctattgctgggaatgttgtaatccgtgattatgatcctaaaaaggctcgttccggcatagtggccaaaggacaaaatgaaaaattcatcatttcacctttaacaaatgaacgcattcctgctgataagcttcatgaacacgttcgatacaatactgttgaccctcaatttaaggaacagagagacagggagcaaatggaacgacaagatgaggatccgatcacagcatctggcacggaaatctcacgaaacattgcaaaattagcagagcgacgtactgatattttcggtattggagaaaagggagtagaacaaacaataattggaaagaaattgggtgaagaagagcgtcaattgcctcgttctgaccccaaaacaatctgggatggccaacaagctacaatcgatgccacaacgcgagcggctcaacaagctgtctcccttgaacaacaaataacagaaattcaacgtcaacatggttatttaccaaatccagctgctgatcgttttgcgccaagtacaccgagtacaactgcatcagcgacaacagcttcgaccactactccaatgccaccaccagcaacaacatctgtgccgactccacttccacctcgccctccacacttgcaaccaccaattgtctcaaatgttgccaaaattatacctggcattccaccacaacaacaaatgcgcatgccacgtatgccattacctgggatgcctccacacatgcttccaatgggcatgcaaatgcccggacttcctccacacatggcaatggcccatcacccgcaagcagggtttatgcctccaccacccccacctgatgaaagtggagcttccgctgcaaagaaattccgcgtagatgatgctttggaaccagaagagctttggcttcaaaaagtgtccggccaaataactgtaaagttgagcacaccacatacagatgaatggaatcttaaaggacaaacatttacaatacaattggagattacttcgactgtaagcgcactcaaatcgctaattcaagaacaaacgactgttcccgcatcgaaggccaagcttgtctatcagaatacgtttctcaaagataattttactttggcctattataattttgtgaatgaagcaaatgttcagttaatgttgaaagagcgtggtgggaagaaaaaatag

SEQ ID NO: 192 - RNA, 1128 nt, Meloidogyne incognita
  source 1..1128: mol_type = transcribed RNA
atgtcaaaggcaacgtatggaccaaaacccgagtcttttgtgttggacgatgggcagcggtattatgttggtgctgatgttggaatgtacctcaaatatcatcgtggaaccctttacaagcgctatcctcagctttggaaaagaatggcctctgtagatgaaaagaagaaaattcaagaattgggatgcgcgacttcctatattaattctaatattatgctggtcaaagctagtgaagtagaagaaatctttgaaggccaagaagagaaatatcgctctggaggtgttggctctggaaattatagccgttcagatgccgcaaatattaaaaatcgtggaggtccaaccccttggcttacacaacaggtcaccagtggttctcatcatctcgaatctgtcccttgctccactccatcggcccatagacgtggacattttcgaaatcgcgattatgcttatgctgccgatgacttggaaaattataaacgtgttttgcaaaatgcagagactgctgaatccttggttccgattaggcttgatatggagttggacaatattaaacttcgtgactatttttgctacaacaaaaacgacaaattaatgacccctgaaatgattgctgaagttctttgtgaagacttggatttgcccttaaacacatttttgcctgcaattgcccaatctatacatcaacaaattgaagcacattctgaagctgtacctgtagatgaacaaactactgatcaacgagctcttttaaaattgaatatacatgttgggaatcaatctctagtagatcaatttgaatgggatatgagcgatccaaaaaatagtcctgaagaatttgggcaaaaattgtctgcagaattgggtttgggtggagaatttgttgctgctatagcttattctattagaggtcaattatcctggaatcaacgtacttatgcatttagtgaatctccattaccaaccgtagaatgtccatttcgtaatcctagcgatgcagaacaatgggggccatttttggaaactttaactagcgcagaaattgaaaagaaaatgcgtgatcaggacagaaatactcgaagaatgcgtcgtttagttaatgcaattccttatattccttga

SEQ ID NO: 193 - RNA, 1128 nt, Meloidogyne incognita
  source 1..1128: mol_type = transcribed RNA
atggacgaagaagtagctgctcttgttgttgataatggctcaggcatgtgcaaggctggatttgcgggtgatgatgctcctcgtgccgtttttccaagtattgtcggtcgtcctcgacatcaaggagttatggttggaatgggtcaaaaggattcgtatgtgggagatgaagctcaaagcaaaagaggtatcttgactttgaaatacccgattgagcacggaattgtaacaaactgggacgacatggagaagatttggcatcatacgttctacaatgagttgcgtgttgctcctgaagaacatccagttctccttacagaggcacctctcaacccaaaggctaaccgtgaaaagatgactcaaatcatgtttgagacattcaacacgcctgccatgtatgttgccatccaagccgttctttctttgtatgcttctggacgtactactggaattgttcttgactctggagatggtgttacgcacacagttccaatttatgaaggttatgctttgccacacgcaatcttgcgtcttgacttggcaggacgtgatttgaccgactacttgatgaagatcctcactgaacgtggttattctttcaccacaaccgccgaacgtgagattgtccgtgacatcaaggagaagctttgctatgttgctttggactttgagcaagagatggctacagctgcttcgtcttcttctttggaaaagagctatgaattgcctgacggtcaagtcatcactgttggaaacgagcgcttccgttgcccagaatctctcttccagccatctttcttggggatggaatctgctggtattcatgagacttcttacaactcgatcatgaagtgcgacattgacatccgtaaagacctttacgccaacactgtcctttctggaggtacaaccatgtatccaggcattgctgatcgtatgcagaaggaaattactgctttggcaccatcaacaatgaagattaagattattgcacctcctgagcgtaaatactctgtttggattggaggatctatccttgcttcattgtctaccttccagcaaatgtggatcagcaagcaagagtatgacgaatctggtccatctattgttcaccgtaaatgcttctaa

SEQ ID NO: 194 - RNA, 516 nt, Botrytis cinerea betaendornavirus 1
  source 1..516: mol_type = transcribed RNA
ttcgttccaggagttacacgcaaagatcacttcactggtggaccagcaaaaagatagtaaacatatcgggaaaatatcgatatatattctccgaattctacgagatatcagagcagaattacctagtaaccctgcactacaaaagtttggactctcacttgtctcatcattcgtgctctcgtcgaaagcaaagacaataaagaagaacaaatggagcgcgagttggagggattgcgaagaggaagtgttagcaatcctactacgcatcgtactagtgccatgagcagcggaactgtgactcaggataggaattctctccaagacaataagagcacagttgtaagctggacgttgttcaaagtgcagtggcaaagcgagcacttgaaatgcagttaaagaatgagggactagatgtcactctacaaattgatcaaactcaacaatggttcaatactttgtgggccgacaatggtgacgccatttctaagcaatacgcttctacagcagcattgaagg

SEQ ID NO: 195 - RNA, 2628 nt, Botrytis cinerea betaendornavirus 1
  source 1..2628: mol_type = transcribed RNA
atgtctaccacaagaatctcaacaccgaaaaggtcccccaaaaaatcgacttttgtcaaaactggaatcttgaccaccaaatcaacgcccaatctcaacgcctcctataatttggcattactacaagcttcaggagctacacccgttcctgcatatccttccaataacggtcaaagttttgccctaaataatcctaggtcgcaaccgtctcgacaagtctcactcgcttcccttacctcgaattcacttgcgacaatcccggatgcaagcaagagataccctctttctacagtctttgatgaggatatgccaacagtaggcaacatgccgccatacacacctgctcgagttggcggtggaccggaagaactagaggttggtgatatagtcgatgtgccaggtaacatgtatggtatcgtcaaatttgttggcagtgtgcaaggcaaaaagggtgtatttgctggggtagaattaagtgaaacgtttgcttcgaaagggaaaaacaatggcgatgtcgaaggaattcaatactttgacacaaccatcgatggtgctgggatttttcttccagtcaacagggcgaagagacgtagcaccccttcgtcgcatgatgagtcatttcccctttcaccggcgtctccatcgatgggcaatagggctgggagattaggatctgaattaaatggtcagccaacacctttgttaccaaaattcggtcaatctgttggtccaggcagagcggcaaacccatatgtccaaaaaacacgtccatccatggctacacctaccacctcaagaccggaatcaccagttcgaagagcagccaatgccaacccatcattaaatacacctgcacaaagagtcccatctcgatatgcaagccctgcgcaggcaaactttggacagagcgttagaggaacacaagattctagagatccaagtaagaaagttggctacaccccccgaaatggcatgaaaacaccaatacctccacgaagtgtttctgcacttggaacggggaatagacctgcaccaatgaactcgatgaatttcagtgatgaagagacacctcctgcagagattgcacgtacggcaacaaacggaagcgtaggctcagtctcttctttcaacgcgaaattacgtccagcatcaagatccgcatcgcgtacaacttccagggctaccgacgacgaatttgagcgattgagaagtttgttagaagatcgcgatagggaaataaaagaacaggcttctattatagaagacatggagaaaactctcagtgaagcacaatcgttgatggagaacaataacgagaacgcaagtggtagacatagtcagggaagtgtggatgacaaggacgcaacacagttgagagcaataatacgtgaaaagaacgacaaaatcgccatgctgactgccgagtttgatcagcatcgagctgatttcagaagcacgatagacacgctcgaaatggccggtgcggaaaccgagcgagtgtacgacgagcgcatgcgtgttctcgtaatggagctcgatacaatgcacgagaatagtcatgatgtaaagcacgttgctgtacaactgaaacagctagaagagctcgttcaggagctcgaggaaggtcttgaagatgcacgacgtggtgaagccgaagctcggggagaagttgagttcttgcgtggagaggttgaaagaactcgatctgaactccgccgcgagcgagagaagactgccgaagctcttagcaacgcaaattctcctacgagcgcaagtgcggaaacacattccaaagagattgctcagagagatgacgagattcgtggattgaaagccatcatccactcgctcagcagagatgccatacctgatgggaatttctcggatcatgaggcaacaccaaatattctacgacctggactaaaccgaagtcgaacagaaagtgcttcggtttctgaggaggagcgccgtactcgggaaaagctagagcgagaagtgagtgagcttcgtgctctcgtcgaaagcaaagacaataaagaagaacaaatggagcgcgagttggagggattgcgaagaggaagtgttagcaatcctactacgcatcgtactagtgccatgagcagcggaactgtgactcaggataggaattctctccaagacaataagagcacagttgtaagctggcgagaacgtggtgcctcagatgctcgccgctacaatctggattcaatgccagagaatgacagctactcctctgcagctgaggatttctgtgaattatgcgaaacctcaggtcatgatgttctacattgcccgatgtttggccccaatggtaacagcagcaattctaaggatgagtcacctaaacagcaacgaacaggaaaagacgttgtcatggagggacttaaattatcacccaaaccttctcaagaagaatacaaaccggcgccgttagcgccagctaagaagtcgcctgatgcgtcgcctatcaagactgttcccaaccttatggaaccaggacctgccccaggaaaggaaagtggagtaatcaacatggataaatggtgcggtgtatgtgaaagagatggacatgacagtattgattgtccttttgaagatgctttttag

SEQ ID NO: 196 - RNA, 2892 nt, Botrytis cinerea betaendornavirus 1
  source 1..2892: mol_type = transcribed RNA
atgcctggcctcgttcgcaaactccttatctttgccgccatcgatgggttgattttgcaaccagcagcgccaaaaggccaacgccccgcccccgcaacgaagatcgcatacaaagataagcatatcgggccagtattgagtgatttgcaggatctggaggggtcgtctgcgaaaagtttcgaggcatttggtattgtcggtctcttgacggtttccaaaagctccttcctgatatcgattacgaaaagagagcaagtcgcacaaatacaagggaaacctatatatgttattactgaagtggctttgaccccattaagttccaagaacgaagcagagatctcgattgatagtacgaaagcggggttattgaagagtaatatcgaggggcagcatggcttggacgagagtgatagcgaggatgatgtcgttagcgatgaagtggaggacgatacagcagtagaagcacacaaaagaacgagtagcgtagctgaagatgtgatctcgaagaaggggggatatggaagatttgctcaaaaatggttctcgaagaaaggatgggccgtggaccagaagaagaacctggggatgagcgctgagccgtattccacagtggagcaagcttccaaggccaccgatgtaccagctacgatttcaggagtcactgaaggaaaatctgatatctcaattcccgataagggcaaggaaattgaggacattgaaactcctgaaaatattagcgacattgcagagagcatgctgccaaaattactacgaacatcgcagatattgtttggggcctctcggagttactacttttcttacgaccatgatatcacaagaagtttggcaaataagaggaatacaaattctgaattgccattgcacaaggaagttgatccactcttcttctggaatcggcatcttactttaccatttattgatgctggccagtcttctcttgccttgcctcttatgcagggctttgtaggacagcgtgcattttcaatggatagtaatccaccaaaccctgctataggttcagacactggaaagacttccgtgcagatgaaggatattacaacaagtagttcggatgagcaaatttacacagcacgtgctggtacagacaagtcgtatctattgacgttaatatctagaaggtcagtcaaacgtgccgggcttagatatttacgccggggtgtggatgaggacggcaatacagccaatggcgtggaaacagagcaaatcttatcggattctgcttggggcccttcgagtaagacatattcgttcgttcagatacgtggcagcattcccatattcttctcccagtcaccttactcttttaaacctgtacctcaagttcaccactctaccgaaacaaattatgaagctttcaagaagcattttgataatataagtgatcgctacggggccattcaagtggcttccttggtggagaagcatggaaacgaggcaatagtcggtggagagtacgagaaattgatgactctccttaatgtctcccgagctagcgagcttaggaaatccattgggtttgaatggtttgatttccatgctatttgcaaaggtatgaaatttgagaatgtcagcctgctcatggaaatactggacaagaagcttgactcgttttcgcacactgttgaaaccgatgggaaacttgtatcgaaacagaatggcgttttaaggactaactgtatggattgtctggatcgaacaaacgttgttcaaagtgcagtggcaaagcgagcacttgaaatgcagttaaagaatgagggactagatgtcactctacaaattgatcaaactcaacaatggttcaatactttgtgggccgacaatggtgacgccatttctaagcaatacgcttctacagcagcattgaagggagactttactcgtactaggaagcgggattataagggggccatcacagatatggggctttctatctccagattttatagcggcattgtaaatgactacttcagtcaagctgccattgatttcctgcttggaaatgtgagctatcttgtttttgaagacttcgaggcaaacatgatgagcggtgatcctggcgtttcgatgcaaaaaatgaggcaacaagccattgatgtttctcagaaactcgttgttgctgacgaccgtgaagaatttattggaggatggacatttctcactccgcaggtacccaatacgatcaaatctagtccttttgaggaatccgtcctcctattgacagatgctgcattgtatatgtgcaattttgattggaatatcgagaaagtatcatctttcgtgagagtggacttgaaccaggtgaacggcatcaagtttggaacatacatcacgagtactttgtcacaagcccaggcagatgagaagaggaatgtgggctttgtaataacttataaggctggttcaaacgacattattcgcgtgaacacgagatctatggctacggaatttccttcttcgaaactctctctcgaagacaaaacatccacgcccgcttctacatctaccaccaactctgtcgtcgccccaattgccgccgggtttgcaaacctaatctcaggtttacaaaatcaaagtatagcggaacctaaagatctcgtgaaggttctcgcattcaaggctctaccctccagatctgcggtatcagatgaaggagttagtgaggccgagcaagtgaagagtgtctgtggagagattagaagaatggttgagattggaagtataagagaggctggagaggagagaaaggatattgtagaggagggtactatcattagtttggccgaggccaagaaaagcacgggactattcgatgtgctgggacatcaggtgaagaaactggtttgggcttaa

SEQ ID NO: 197 - RNA, 1608 nt, Botrytis cinerea betaendornavirus 1
  source 1..1608: mol_type = transcribed RNA
atgggattaccaccatgggagacaagtgagagccagacttttgccttaataacaggcgcaaacagcggcttgggttttgccattgcttctagattgattgacgaattcctgacttcatctgatactcctcctactaaacatttgattctcatcctttgcacgcgtacacctctgaagacacgattcacaatctcccgcctgcgtgctcatctacgaaaactggccgactactctgaattcgcaacttcacatcgagcgaaagctaagtctcagggaaatgtatatagatggcaggatacggtggctcgggttcattttcttggggttgaagttgacttgtgcgacttgaaaagtgtatatgctctcaccgataggcttgtcaatggaactgttggtagcccagatgctactacaatggatggtctccgattacctgatggctctcctggtaccgccacttattccgcagatgttaaacaagacagatgggcattgagtcagaaggaagggtctgaaggcgagctgagatcttggggatggggattgagcggaattaggataccaaggctggatgttgtagtcttgaatgcagggataggaggatggtcaggaatcgattggccaaaggctatatggactgtcataacagatatgacggaagctgtaacatggccaacttacaagcttgcagaaattggagttatcaccaaacctcaactatcctctgcctcttcaaaacaagccaaaccctccgacgatgaagctgcgcaacctttactcgacggtgaatctccggaagaaccacctcttggagcaacattctgttctaacgttttcggacattacgttctcgcacacgagctgatgcctttactatcgcgaactgcttctccttcggccactaccagcggaagaataatatgggtgtctagtatcgaggcacaggcacaacattttgatgaagatgatttacaaggtttggaaagtcgcacgccatacgaaagctccaaacgtcttacagatgttctcgtcctttcaagaaaattacgagcagcaggccaagcctcctcttcatggttcgattgttctgacgttcgcgtggagaactctaaatcggaggaagaaccagcagcaaaattgataagaccgaaaatgtacgtggttcaacctggaatttttgtttcggagatcatgcccttgaattttgtgttggtatttatctacagattgatcatctaccttgtaagatggatgggaagccaatggcataccatcaagccttacactgctgctgtagcaccagtctgggtagcattatctcccgacgatgttctagataacatggacggggcagcttcgaaatggggatcagctacagatgctagtgggaaagaacgcgttatacgaaccgaagttcctggttggggatgggaaggcaaaactccaaagacaattgatactggagagagaagaaaggggaggcaaagaaatgcggaaccattgacgagggaagcgagagaggattttgaggtcttgggtgttaagtgttggacggagatggagaatttgaggaaagaatgggagggacttttgaaggttaaaaaatga

SEQ ID NO: 198 - RNA, 2604 nt, Botrytis cinerea betaendornavirus 1
  source 1..2604: mol_type = transcribed RNA
atggtcttgttggtatcaaaaagagggtgtcgagtatggcatcgatacttgacacaagttacatggccttgtctggctaagcctagtcaattgaatgcgttcattcgttatagttcacagcagactgaaagtcatgatacgttaacagagacaactcgaaatattggcatcatagctcatatcgatgcaggcaaaactacaactactgagcgtatgctctattacagtggctatactcgacgcataggagacgttgatgatggctccaccgtcacagacttcctccccgccgagagggctagagggattactattcagtcggcagccgttacgatgcactggccccctgtgactgagaaagcagcatcctcgaaagcgtctgacccatctcgatcacttatatcgcacactcttaacctgattgatactcccgggcacgcggattttacgtttgaagtgcttcgttctttgagaatccttgatggtgccgtatgtattcttgatggtgtggcaggagtagaagctcaaacagaaaaagtatggaaacaggcaaataattatagcattcccaggatcgctttcgtcaataagcttgatcgtgaaggggctgccttcgcgagaacagtaaaagagattgcttcgaggctccggggctggcctgcggtgtgtcaaattccatggtgggaaggtgggaaaggaaagtttacgggagtcggtgatgcagtcaatctttgtgcgctgaaatgggaagaaggtggggatggcaaatcaatccaacattttaccctcgatcagctgaggaaagataatcctgaattttccgaagaaatcatcaaggcgcgagctgcgctcgtagaggcattatgcgaatttgatgatgggctccttgaaacctggctagaatgtaatgatgacagtctgcttatatcatcagcggacattacaaagagtttgcgaaaatgcgtactcgacggttcgggtaaactcatacctgtctttgctggggccagttttcgaaatattggcgttcaaccactccttgatgccatcaataatcttctacccaaccctaccgaaaggcctgaaccagaagttcgacttggcaatgttcatggcagtttatcccaactcctagatggaaagctggaccttagcgcagcgcaaccaagacatctctccaagaaatccaatgcgcgtacgtctctcatttctcaggtcgaagcttgtgcccttgctttcaaggtagtcaatgatgctcgacgcggcgtgttagtctacatcagagtttattccggtacagtcaagcggaacactactttatggaatacgaatcttcatgtttcagaaagagcacaaaggctgttacagatgtacgcatctgacgcggtagagatacaaagcattccctccggtcagattggcgttattgcgggcttgaagcacgctcgtacaggcgatactctattgtcatatacaggtgctcatccaaaaaatggcccgccaccacctttgaatactcttcaactgagaccaattaatgttcctccgccagtattttttgccgctatagaacctcacagtctaagcgaagaaaaaaatgttgccgacatattgcagctcttattgagggaagacccaagtctccatgttaatattgatgaagaatctgggcaaatgcagttgagtggtatgggtgagctgcatcttgaaatcgcaaaagatcgactcgtaaacgatttcaaagccaaggcaaccatgggcaatatcgaaattgggtaccgagaatgcgtcctgtctccaacattacctcacaaatatatatttgatcgtgaagttgctggcaaaaaaggaaaagcaagctgcgaagctagtatcagtccagacgcgcagccgttagaagattctgaccacactatgaacattgatggaaatacaatcacaatccgtatcccaaaaccttctaacaatgatggggttgatggaactatgacactcccctccgatatgaccctcagcgaagttcatacagcattgatcaatggggctgttgctggtctagcccgtggtccgcgtcgtgcattcccacttcattctgcgcatgtgctattgaagtttgataccgttgaggatgtctttggttccgacactacttctgccgcattgtcttctgcgtcaagaaatgccgtacaagctgctctcaaagaagcatttcaagcgaactccatcggactgatggaaccagtcatgaatgtggttattagttgcgacgaatcatccctcggtgcaattgtgcacgatctatcatcagctcgtggtggccatgtcttatcacttgagaatgatagtgacgtagaggatacagaaaatgagctggcgccagttgatgtttcaagagtctatactcctccagatccattcgcgtcaggaaccagtactgattcatcgggtcctggccagcagcgtcaaatcaccgctagagtaccattgaaagagatggttggatatttgaagcatttgaggagcatgactggcggtagaggaacattcgtcatgagcgttgataggtttgaaagactttctgggccaagagaaaaggctttgtag

SEQ ID NO: 199 - RNA, 2793 nt, Botrytis cinerea betaendornavirus 1
  source 1..2793: mol_type = transcribed RNA
atgtcctataatcgattagatccggcgggccaaggccaagacgattattacaatatgaataatagacatcaatcgccgcaccctcaagggtaccaactagaagatgcgccatatggcagaccaaatactacttcacccggtcctggaatgcacaatctagagatacctatgggtcctggggcacatagaattggaacgccgagtgatcaattgcaagctcaaccatcttattcggtcgaacatttagatcaaaatcaataccatcagcgcatgcccctcaatcccagtcaaagctacgactcggaatattctttagatcctaacgcgcatcacgatgcttactaccaacctccataccaaccctctcctcatgaagaacaccctctacagaactatgccccagggcaagacccctacgcttacaatgatgatgatgaccatcaaccaattctacaatcgcatgaaccttatgggccagatccgcactcagctagtggcactgattacaaaggtggttatgacgggacggtacaatctccatcagcgacacctgtacctgcgttaagaagatacaagacggtcaaggaagtccaactgttcaatggaaatctcgtactcgattgtccgattcctcccaagcttttaaatcaagtcaatcatgcaccaccgccggagagagatgagttcacacatatgagatattcagccgcaacatgcgacccctctgagttttttgaggagcgctttacattgcgacagaaactcttcgcaaagcctcgacacacagaactcttcatcgttgtcacaatgtataatgaagatgatgtattgtttgctagaacaatgcacggagtattcaagaacattgaatttatgtgcactcgtaaagacagtaaaacatggggcaaggatgcttggaagaagatcgtagtgtgtgtggtcagtgatggacgtgccaaaatcaatccaagaacaagggctgttttggctggtttgggggtttatcaggacggaattgcgaagcaacaagtcaatggcaaggacgtgacagcacacatctatgaatatacgactcaagtcggaatttctttgaaaaaggatattgttacactcacaccaaaacagcaacccgttcaattgcttttctgtttgaaggaaaagaatcagaaaaagatcaactctcacagatggttcttccaagcctttggtagagtacttgatcccaacatttgtgtactcctcgatgctggtacaaaaccaggaaaggactcaatttatcatctatggaaagctttcgatttggaaccacattgtgcaggagcttgtggtgagatcaaagctatgttgggcccaggaggaaagaatttggtcaatccattggttgctacccagaatttcgagtacaagatgagtaatattctggacaaacctcttgaatcggcatttgggttcatttcggtgttgccaggtgctttttctgcatatcgatatgtcgcgttacaaaacgataaaactggaaatggaccactggagaagtactttgcaggagagaagatgcatggagcaaacgcgggtatttttactgccaacatgtaccttgccgaagatcgtattttgtgtttcgaactggtatcaaagagaaattgtcactggatcttacaatacgtcaagtctgctactggagaaaccgatgtaccagatacaatggcggaacttattttgcaacgtcgaagatggcttaacggaagtttcttcgctgctatctatgctcttgctcatttttatcaaattttccgaagtgatcacagctttttacgaaaaatcatgtttttgattgaattcacatatcaaactattaacatgatcttcgcttggttcgctcttggtaatttcttcttggtttttcacatcttgactacatctctcggaagttctgacttactgggtaacgttggtgtaattcttggtgttgttttcgaatggctttacttgtttacgcttttgacctgttttattctggcgctgggaaatcgacctcaaggaacgaacaaagtttatatgtctatggtttatttctggatcattatcatgatatacctaatgtttgcttcgattttcatcacggttaaatcaatccaaactcaacttgctaaagacgaattcaactggacggatattatcaagaatcagatcttctatactctgatcatttctctcgcctcaacttatctactctggttcatttcttcctttttattcttcgacccatggcacatgtttacatctttccttcaataccttcttctcactccaacatatatcaatattctcaacgtttacgctttctgtaacactcacgatattacatggggtacaaaaggagatgacaaagccgagaaacttccatccgctgtgctcaaagctggtggtaaagtcgacgtcaatattcccaccgacgacggtgatctcaacgcccaatatgaatcggaaatgcgcaagattcagatcaaagctccacctgagaaacgtgaattctcggcagaagagaagcaagaggactattataaatggttcagatcttcggtagtgttgttctggttggtttgtaattttgcactcacgagtgcggtgcttagtacggcgggcttggaaaaatttggtaccggtactgatgatacgacgaagagtacgatttacatggctgtggttttatggagtgtggcaggattgagttgtttcaagtttattggtgcgatgtggtttttgattgttaggatgtttagaggtgtttag

SEQ ID NO: 200 - DNA, 766 bp, synthetic construct
  source 1..766: mol_type = other DNA
ggactagttaatacgactcactatagggcgaattgggtaccgggcattacgtttcttaattgctgtatactcttcttatgatacgttatcagatttttcaaggtaatctatagtctgcactttcgacgaaagagtaagaacttcgaacaaaaaagcgacaaggagctcatcatggtatgttctagcgcttgcaccatcccatttaactgtaagaagaattgcacggtcccaattgctcgagagatttctcttttacctttttttactatttttcactctcccataacctcctatattgactgatctgtaataaccacgatattattggaataaataggggcttgaaatttggaaaaaaaaaaaaaactgaaatattttcgtgataagtgatagtgatattcttcttttatttgctactgttactaagtctcatgtactaacatcgattgcttcattctttttgttgctatattatatgtttagagtgatgagctccttgtcgcttttttgttcgaagttcttactctttcgtcgaaagtgcagactatagattaccttgaaaaatctgataacgtatcataagaagagtatacagcaattaagaaacgtaatgcccggtacccaattcgcccggccggcatggtcccagcctcctcgctggcgccggctgggcaacattccgaggggaccgtcccctcggtaatggcgaatgggaccctagcatatccatgatatctgttagtttttttcctgaaaggtctagagc

SEQ ID NO: 201 - DNA, 942 bp, synthetic construct
  source 1..942: mol_type = other DNA
ggactagttaatacgactcactatagggcgaattgggtaccgggcattacgtttcttaattgctgtatactcttcttatgatacgttatcagatttttcaaggtaatcttgatgagctccttgtcgcttttttgttcgaagttcttactctttcgtcgaaagtgcagactattctcagatctgccggtctcccggccggcatggtcccagcctcctcgctggcgccggctgggcaacattccgaggggaccgtcccctcggtaatggcgaatgggaccctagcatatccatgatatctgttagtttttttcctgaaagaatgttctagcgcttgcaccatcccatttaactgtaagaagaattgcacggtcccaattgctcgagagatttctcttttacctttttttactatttttcactctcccataacctcctatattgactgatctgtaataaccacgatattattggaataaataggggcttgaaatttggaaaaaaaaaaaaaactgaaatattttcgtgataagtgatagtgatattcttcttttatttgctactgttactaagtctcatgtactaacatcgattgcttcattctttttgttgctatattatatgtttaatacgactcactatagggagaccggcagatctgagaatagtctgcactttcgacgaaagagtaagaacttcgaacaaaaaagcgacaaggagctcatcaagattaccttgaaaaatctgataacgtatcataagaagagtatacagcaattaagaaacgtaatgcccggtacccaattcgcccggccggcatggtcccagcctcctcgctggcgccggctgggcaacattccgaggggaccgtcccctcggtaatggcgaatgggaccctagcatatccatgatatctgttagtttttttcctgaaaggtctagagc

SEQ ID NO: 202 - DNA, 760 bp, synthetic construct
  source 1..760: mol_type = other DNA
cccaagcttggggtgcttcggatgctgatgagtccgtgaggacgaaacagggcaacctgtccatccggtatcccaccgggcattacgtttcttaattgctgtatactcttcttatgatacgttatcagatttttcaaggtaatctatagtctgcactttcgacgaaagagtaagaacttcgaacaaaaaagcgacaaggagctcatcatggtatgttctagcgcttgcaccatcccatttaactgtaagaagaattgcacggtcccaattgctcgagagatttctcttttacctttttttactatttttcactctcccataacctcctatattgactgatctgtaataaccacgatattattggaataaataggggcttgaaatttggaaaaaaaaaaaaaactgaaatattttcgtgataagtgatagtgatattcttcttttatttgctactgttactaagtctcatgtactaacatcgattgcttcattctttttgttgctatattatatgtttagagtgatgagctccttgtcgcttttttgttcgaagttcttactctttcgtcgaaagtgcagactatagattaccttgaaaaatctgataacgtatcataagaagagtatacagcaattaagaaacgtaatgcccggtgggataccggatgggccggcatggtcccagcctcctcgctggcgccggctgggcaacattccgaggggaccgtcccctcggtaatggcgaatgggactctagagc

SEQ ID NO: 203 - DNA, 900 bp, synthetic construct
  source 1..900: mol_type = other DNA
ggactagttgaagtttttctcggcgagaaatacgtagttaaggcagagcgacagagagggcaaaagaaaataaaagtaagattttagtttgtaatgggagggggggtttagtcatggagtacaagtgtgaggaaaagtagttgggaggtacttcatgcgaaatgggtaccgggcattacgtttcttaattgctgtatactcttcttatgatacgttatcagatttttcaaggtaatctatagtctgcactttcgacgaaagagtaagaacttcgaacaaaaaagcgacaaggagctcatcatggtatgttctagcgcttgcaccatcccatttaactgtaagaagaattgcacggtcccaattgctcgagagatttctcttttacctttttttactatttttcactctcccataacctcctatattgactgatctgtaataaccacgatattattggaataaataggggcttgaaatttggaaaaaaaaaaaaaactgaaatattttcgtgataagtgatagtgatattcttcttttatttgctactgttactaagtctcatgtactaacatcgattgcttcattctttttgttgctatattatatgtttagagtgatgagctccttgtcgcttttttgttcgaagttcttactctttcgtcgaaagtgcagactatagattaccttgaaaaatctgataacgtatcataagaagagtatacagcaattaagaaacgtaatgcccggtacccatttcgcatggccggcatggtcccagcctcctcgctggcgccggctgggcaacattccgaggggaccgtcccctcggtaatggcgaatgggacctctataaattttatttgtcttaagaattctatgatccgggtaaaaatctagagc

SEQ ID NO: 204 - DNA, 1210 bp, synthetic construct
  source 1..1210: mol_type = other DNA
ggactagttgaagtttttctcggcgagaaatacgtagttaaggcagagcgacagagagggcaaaagaaaataaaagtaagattttagtttgtaatgggagggggggtttagtcatggagtacaagtgtgaggaaaagtagttgggaggtacttcatgcgaaatgggtaccgggcattacgtttcttaattgctgtatactcttcttatgatacgttatcagatttttcaaggtaatcttgatgagctccttgtcgcttttttgttcgaagttcttactctttcgtcgaaagtgcagactatgggagaccggcagtttcgcatggccggcatggtcccagcctcctcgctggcgccggctgggcaacattccgaggggaccgtcccctcggtaatggcgaatgggacctctataaattttatttgtcttaagaattctatgatccgggtaaaaaatgttctagcgcttgcaccatcccatttaactgtaagaagaattgcacggtcccaattgctcgagagatttctcttttacctttttttactatttttcactctcccataacctcctatattgactgatctgtaataaccacgatattattggaataaataggggcttgaaatttggaaaaaaaaaaaaaactgaaatattttcgtgataagtgatagtgatattcttcttttatttgctactgttactaagtctcatgtactaacatcgattgcttcattctttttgttgctatattatatgtttgaagtttttctcggcgagaaatacgtagttaaggcagagcgacagagagggcaaaagaaaataaaagtaagattttagtttgtaatgggagggggggtttagtcatggagtacaagtgtgaggaaaagtagttgggaggtacttcatgcgaaactgccggtctcccatagtctgcactttcgacgaaagagtaagaacttcgaacaaaaaagcgacaaggagctcatcaagattaccttgaaaaatctgataacgtatcataagaagagtatacagcaattaagaaacgtaatgcccggtacccatttcgcatggccggcatggtcccagcctcctcgctggcgccggctgggcaacattccgaggggaccgtcccctcggtaatggcgaatgggacctctataaattttatttgtcttaagaattctatgatccgggtaaaaatctagagc

SEQ ID NO: 205 - RNA, 21 nt, synthetic construct
  source 1..21: mol_type = other RNA
tagttcatccatgccatgtgt

SEQ ID NO: 206 - RNA, 21 nt, synthetic construct
  source 1..21: mol_type = other RNA
acatggcatggatgaactata

SEQ ID NO: 207 - RNA, 21 nt, synthetic construct
  source 1..21: mol_type = other RNA
aagttcatccatgccatgtgt

SEQ ID NO: 208 - RNA, 21 nt, synthetic construct
  source 1..21: mol_type = other RNA
acatggcatggatgaacttta

SEQ ID NO: 209 - RNA, 1727 nt, synthetic construct
  source 1..1727: mol_type = other RNA
gaaccagatcggtaccgctagcaagatctcctaggaagctttccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagaattcgatatcatcgatctcga

(19) United States
(12) Patent Application Publication
(10) Pub. No.: US 2026/0174085 A1
(43) Pub. Date: Jun. 25, 2026

(54) NUCLEIC ACID ACTIVE AGENTS AGAINST VARIOUS PLANT PATHOGENS

(71) Applicant: Martin-Luther-Universität Halle-Wittenberg, Halle (Saale) (DE)

(72) Inventors: Aysan GHASEMZADEH, Ardebil (IR); Marie KNOBLICH, Leipzig (DE); Sven-Erik BEHRENS, Halle (Saale) (DE); Selma Persida GAGO-ZACHERT, Halle (Saale) (DE); Torsten GURSINSKY, Halle (Saale) (DE)

(21) Appl. No.: 19/128,143

(22) PCT Filed: Nov. 7, 2023
(86) PCT No.: PCT/EP2023/080990

§ 371 Date: May 7, 2025

(30) Foreign Application Priority Data
Nov. 7, 2022 (EP) 22205810.9

(51) Int. Cl. A01N 63/60; A01P 1/00; A01P 3/00; A01P 5/00; C12N 15/113

(52) U.S. Cl. CPC A01N 63/60; A01P 1/00; A01P 3/00; A01P 5/00; C12N 15/1137; C12N 2310/14

(57) ABSTRACT

The invention relates to newly identified nucleic acids, ribonucleic acids (RNAs) and deoxyribonucleic acids (DNAs), specifically esiRNAs/ERNAs (effective small interfering RNAs) and RNAs derived therefrom, as well as eASO (effective antisense DNA oligonucleotides), collectively referred to as eNAs (effective nucleic acids), which can be used in RNA silencing/RNAi or RNA silencinglantisense methods, as active agents against various variable plant pathogens. To identify the eNAs, an “eNA screen” was applied for the first time in standardized form to target RNAs of various plant pathogens. As a result, a new class of active agents against pathogens was identified and successfully used. The invention further relates to the construction of double-stranded ribonucleic acids, edsRNAs (effectively double-stranded RNAs), which contain nucleotide sequences of identified esiRNAs/ERNAs or related RNAs derived therefrom, and which can be successfully used in the RNA silencing/RNAi methods as active agents in plant protection against variable plant pathogens.

### CROSS REFERENCE TO RELATED APPLICATIONS

The present application is a U.S. National Phase of International Application No. PCT/EP2023/080990 entitled “NUCLEIC ACID ACTIVE AGENTS AGAINST VARIOUS PLANT PATHOGENS,” and filed on Nov. 7, 2023. International Application No. PCT/EP2023/080990 claims priority to European Patent Application No. 22205810.9 filed on Nov. 7, 2022. The entire contents of each of the above-listed applications are hereby incorporated by reference for all purposes.

### SEQUENCE LISTING

The material in the accompanying sequence listing is hereby incorporated by reference in its entirety into this application. The accompanying file, named Sequence Listing_MAI25303PCTUS, was created on May 2, 2025, and is 210 KB.

### FIELD OF THE INVENTION

The invention relates to newly identified nucleic acids, ribonucleic acids (RNAs) and deoxyribonucleic acids (DNAs), specifically esiRNAs/ERNAs (effective small interfering RNAs) and RNAs derived therefrom, as well as eASO (effective antisense DNA oligonucleotides), collectively referred to as eNAs (effective nucleic acids), which can be used in RNA silencing/RNAi or RNA silencinglantisense methods, as active agents against various variable plant pathogens. To identify the eNAs, a screening method (WO2019001602 A1), hereinafter also referred to as “eNA screen,” was applied for the first time in standardized form to target RNAs of various plant pathogens. As a result, a new class of these active agents against these pathogens was identified and successfully used against them.

The invention further relates to the construction of double-stranded ribonucleic acids, edsRNAs (effective double-stranded RNAs), which contain nucleotide sequences of identified esiRNAs/ERNAs or related RNAs derived therefrom, and which can be successfully used in the RNA silencing/RNAi methods as active agents in plant protection against these same variable plant pathogens*.

The term “plant pathogens” refers here overall to plant-infecting viruses and also plant-infecting organisms such as nematodes and fungi that have a damaging effect on plants.

### BACKGROUND OF THE INVENTION

RNA interference (RNAi) is a mechanism that is best characterized in higher eukaryotes, but is active in cells of almost all organisms. The RNAi mechanism serves to switch off (RNA silencing) or modulate gene expression and, as will be explained below, can also be used in a targeted manner for these purposes (see inter alia Shabalina and Koonin 2008; Carthew and Sontheimer 2009). In the process, cellular factors, which are described in detail in the text below, inactivate various forms of small ribonucleic acid molecules, which are referred to here with the general term small RNAs, “sRNAs,” target ribonucleic acid molecules (hereinafter referred to as “target RNAs”) or modulate the function, for example the translation, of these target RNAs. The term sRNAs includes, for example, small interfering RNAs, siRNAs, and, for example, micro RNAs, miRNAs, but also other forms of small RNAs which can trigger RNA silencing (see for example Borges and Martienssen, 2015; Zhan and Meyers, 2023). The target RNAs originate from pathogens that infect foreign cells or originate directly from a cell that is part of an organism, including an organism acting as a pathogen, in which RNAi is effective.

RNAi probably originated as an evolutionarily conserved cell defense mechanism. RNAi is a major component of the immune response to pathogens in plants, but also in insects, nematodes, oomycetes and fungi (see inter alia Ding 2010; Zvereva and Pooggin 2012; Gammon and Mello 2015; Guo et al. 2019). The RNAi process is further described below primarily for plant cells and in relation to its function as a defense mechanism, as it is one of the best studied, in particular with regard to its antiviral efficacy (schematically shown in FIG. 1). RNAi is also active, with modifications, in a similar form in nematode, oomycete, fungal and insect cells.

RNAi is triggered by ribonucleic acids containing double-stranded (ds) regions, i.e., two nucleotide strands of different RNA molecules or of the same RNA molecule that are paired (hybridized) via complementary base pairing. RNA molecules with nucleotide building blocks which can form double strands over large areas, potentially over hundreds or thousands of nucleotides, are referred to as “dsRNAs.” The induction of RNAi in viral infections has been well studied: triggers can be structured, double-stranded regions of viral messenger RNAs (mRNAs) or viral RNA genomes. RNAi is particularly effectively triggered by dsRNA replication intermediates. These are created during the replication of RNA viruses and consist of complementary RNA double strands comprising hundreds or thousands of base pairs. Double-stranded regions of RNAs can be perceived in the cell as “pathogen-associated molecular patterns” (PAMP) and therefore as “foreign.” Detectors include cellular enzymes belonging to the family of type III ribonucleases and referred to as Dicer or Dicer-like proteins (DCLs) (Fukudome and Fukuhara 2017; Song and Rossi 2017). The proteins DCL2 and DCL4, first characterized in the model plant Arabidopsis thaliana (A. thaliana), play a central role in the plant antiviral RNAi immune response (Deleris et al. 2006; Parent et al. 2015). DCL2 and DCL4 bind dsRNAs and process them into siRNAs through endonucleolytic hydrolysis (cleavage). siRNAs are short RNA molecules 21-25 nucleotides (nt) in length, consisting of two complementary single-stranded RNA components. These “RNA duplexes” are phosphorylated at the 5′ end and at the 3′ end they have a 2 nt-long, single-stranded overhang and 3′ hydroxyl groups (Elbashir et al. 2001a; Elbashir et al. 2001b). siRNAs with lengths of 21 or 22 nt are particularly effective antivirally (Deleris et al. 2006). After generation by the DCLs, one strand from the originally predominantly double-stranded siRNAs (see above), the “guide strand,” becomes active in RNA-induced silencing complexes (RISC). The main components of RISC are “Argonaute” (AGO) proteins; these resemble RNase H enzymes and can also have endonucleolytic activity. The AGOs bind the guide strand (gs) of the siRNA duplex, while the other strand of the duplex (passenger strand, ps) is removed and broken down (Meister 2013; Kobayashi and Tomari 2016) (FIG. 1). 10 different AGO proteins (AGO1-10) are encoded in A. thaliana; they have different functions, some of which are not yet fully understood. However, it has been well demonstrated that AGO1 and AGO2 proteins have a central, essential role in the plant's antiviral immune response (Carbonell and Carrington 2015). The AGO proteins have different binding priorities for siRNA guide strands. For example, AGO1 binds with high priority to RNAs containing a 5′-terminal U nucleotide, while AGO2 prioritizes binding to RNAs having a 5′-terminal A nucleotide (Mi et al. 2008; Takeda et al. 2008; Schuck et al. 2013). After binding of the siRNA guide strands by AGO, the corresponding RISC is associated with the target RNA. The sequence complementarity of the bound siRNA guide strand with the sequence in the target RNA to which it can hybridize, referred to here as the target site, plays an important role (Liu et al. 2014). The sequence complementarity is naturally highest to the target RNA from which the siRNAs originally originate. Accordingly, under natural circumstances, target RNAs are also those RNA molecules (“cognate RNAs”) that were originally processed into siRNAs. In viral infections, these are accordingly viral mRNAs, viral genomes or viral replication products; in other pathogens, these are usually mRNAs. After the AGO/RISC has associated with the regions of the cognate target RNA which are complementary to the siRNA guide strand, which regions can be coding or noncoding (untranslated) regions, endonucleoelytic hydrolysis (cleavage or “slicing”), catalyzed by the AGO protein, of the target RNA can take place (FIG. 1); this takes place between the nucleotides of the target RNA which lie opposite nucleotides 10 and 11 of the siRNA guide strands (Elbashir et al. 2001a; Elbashir et al. 2001c; Wang et al. 2008). Alternatively, there is evidence that siRNA-containing AGO/RISC associated with target RNAs inhibit the translation of these RNAs (Brodersen et al. 2008; Iwakawa and Tomari 2013). The result of the activity of the siRNAs in the RNAi process is therefore in any case a temporary inhibition of gene expression: the target RNAs are cleaved and broken down or the synthesis of the proteins encoded by these RNAs is inhibited. The result of RNAi directed against pathogens is therefore the inhibition of replication of the pathogen by inhibiting gene expression (RNA silencing) (Zvereva and Pooggin 2012). Accordingly, RNAi is already being used in a targeted manner to protect plants from pathogens such as viruses (Khalid et al. 2017; Pooggin 2017).

DsRNAs derived from the sequences of target RNAs of pathogens can accordingly be used as antipathogenic active agents: they are processed by the Dicer enzymes and generate siRNAs, some of which (see below) can then become effective in the manner described above. To achieve an effect against viruses, the dsRNAs have to be used in the plant. Accordingly, dsRNA active agents and the resulting siRNAs produced in the RNAi immune system of plants, nematodes, fungi, oomycetes and insects, which act against these pathogens' own RNAs (usually mRNAs), can be used as herbicides, nematicides, fungicides or insecticides in and on the plant. As an alternative to dsRNAs, sRNAs such as siRNAs, miRNAs or other forms of small RNAs effective in the RNAi process (e.g., piRNAs, tasiRNAs, vasiRNAs etc.) can also be used directly as antipathogenic active agents (see inter alia Tomilov et al. 2008; Banerjee et al. 2017; Majumdar et al. 2017; Price and Gatehouse 2008). As will be explained in more detail below, all of these sRNAs operate according to the same principle as siRNAs: basically, a single-stranded component of these sRNAs is incorporated analogously into RISC and is active in the RNA silencing/RNAi process against a target RNA by slicing or by inhibiting translation.

As already mentioned, RNAi is used for the artificial “knockdown” of gene expression in such a way that siRNAs or other related sRNAs or sRNAs derived therefrom, such as miRNAs, are introduced into cells as active agents in order to influence the function of target RNAs in a targeted manner. For example, as described, cellular gene expression, but also the gene expression of pathogens, can be suppressed or modulated at the mRNA level. In addition, the replication of viruses, the genomes of which are RNA molecules, can be suppressed via the silencing of these genomes.

For the purpose of artificially inducing RNAi, an organism can produce (express) RNA active agents itself. This is already practiced in plants. In this case reference is made to “host-induced gene silencing” (HIGS): plants express dsRNAs, siRNAs or other sRNAs such as miRNAs in the form of a transgene (Herrera-Estrella et al. 2005; Dong and Ronald 2019; Koch and Wassenegger 2021) to achieve protection against pathogens such as viruses, but also nematodes, fungi, insects, oomycetes or other parasitic plants. This can be achieved permanently (and it is only permanently that this process make sense) by stable integration of the respective nucleic acid-expressing foreign gene into the genome of the organism to be protected. Obtaining transgenes is complex, not possible with all plants and unsuitable for many applications. The latter applies in particular to applications against rapidly-changing pathogens: a transgenic crop obtained after years of development may nevertheless still become infected, namely by variants of these pathogens (Jan et al. 2000; Savenkov and Valkonen 2001; Simón-Mateo and García 2006; Tabashnik et al. 2013; Kung et al. 2015). In addition, the use of transgenic plants (genetically modified organisms (GMOs)) in agriculture and horticulture is prohibited in many countries (Lucht 2015).

Alternatively, RNA active agents can be used in “topical”/“transient” applications. This involves dissolved RNA, e.g., in the simplest case in the form of a spray, being applied to a target organ or target cells of an organism. The RNA is partially taken up via mechanisms that have not yet been fully clarified. Improving the uptake mechanisms of negatively-charged RNA molecules into target cells, which are inefficient, is the subject of intensive research. Due to the biodegradability of RNAs, “spray-induced silencing” (SIGS), in which RNA is used in the form of a solution (e.g., as irrigation water or spray) on and in plants, is also ecologically much less critical than HIGS and is therefore attractive for the administration of RNA active agents (Dalakouras et al. 2020). In addition, in the last 5 years or so, methods have been developed that allow the production of RNA on a gram or even kilogram scale (Robinson et al. 2014; Kaur et al. 2018). For example, the price of one gram of dsRNA, which is needed for treatment on a small field of crops, has dropped from over USS100,000 to less than USS2 (Le Page 2017).

As already mentioned above, in the case of plant protection, the aim of topical/transient applications of RNAs is either that plants internalize the RNA active agents, e.g., in order to inhibit viral replication, or that attacking pathogens such as fungi, nematodes, insects or parasitic plants take up RNA active agents via the surface of the plant to be protected and these then become active in the RNAi system of the organisms in question against essential target RNAs of the pathogens (see also above: Tomilov et al. 2008; Banerjee et al. 2017; Majumdar et al. 2017; Price and Gatehouse 2008).

However, the use of RNA active agents in topical/transient applications is not yet fully developed, for various reasons. One reason is the already mentioned inefficient uptake of RNA into target cells. Therefore, intensive work is being carried out on methods to stabilize RNA active agents by chemical modifications of the nucleotide building blocks and/or to deliver them to the site of action in the cytoplasm of the target cells by physical or biochemical methods (Dowdy 2017; Setten et al. 2019; Dalakouras et al. 2020). However, there are major differences from application to application and from organism to organism.

Another reason relates to the fact that both natural and artificially induced RNA silencing/RNAi processes are usually inefficient. This is particularly due to the fact, which has now become well established by a large amount of data, that, while an enormous number (a “pool”) of siRNAs result from target RNAs by means of the Dicer/DCI activities, only a few siRNAs from such a final pool are actually effective on the target RNA (FIG. 1). This is in particular caused by the fact that the target RNAs are usually highly structured and have only a few “accessible sites” (referred to as “a-sites”) for effective association of sRNA/AGO/RISC to the complementary target sites and thus for silencing.

This is especially true for target RNAs from pathogens: the target RNAs of pathogens are often adapted (attenuated) in their structure through coevolution with the target organism so that they can evade silencing/RNAi as much as possible. a-sites are accordingly defined here as regions of a target RNA that are accessible to nucleic acid active agents. An a-site can correspond to a “target site.” A target site is defined here as the sequence of a target RNA to which a single-stranded nucleic acid such as an siRNA guide strand or an antisense oligonucleotide (see below) can hybridize by means of complementary base pairing. An a-site can also contain larger regions of a target RNA, e.g., RNA structural motifs that are particularly readily accessible and contain one or more target sites of single-stranded nucleic acids.

To date, double-stranded versions of target RNAs, e.g., dsRNA versions of mRNAs or viral genomes, have mainly been used in SIGS for RNAi (Robinson et al. 2014). It is important to note here that ALL dsRNAs used for RNAi to date comprise either the complete sequence or a longer uninterrupted section (usually several hundred nucleotides) of the corresponding target RNA on one strand (see also control RNA dsCMV used in FIGS. 9, 10, 11 and 15). The second RNA strand was a second molecule having a completely complementary sequence. Alternatively, “hairpins” are used. In this case, the two complementary RNA strands are located in the same molecule and are separated by a “spacer,” a largely unstructured, single-stranded sequence of any nucleotide composition and length. They thus form an incomplete double strand, a “hairpin.” After entering the plant cell, dsRNAs or dsRNA hairpins are accordingly processed analogously to the normal target RNAs into an siRNA pool by the DCLs.

However, this pool is again subject to the above-mentioned problem: only very few of the siRNAs generated from these “conventional dsRNAs” are effective (see also schematic depiction in FIG. 1 and FIGS. 10 and 15).

“Conventional dsRNAs” means that one strand of these RNAs consists of an exact copy of the targeted target RNAs and that this strand is then hybridized with a complementary RNA strand: as explained, dsRNAs constructed according to this principle are currently used in RNAi-mediated plant protection.

In contrast, the remaining siRNAs of the pool, i.e., the overwhelming majority, can nonspecifically saturate cellular AGO/RISC (they thus act as a “decoy” for the AGO/RISC), which can even lead to the RNA silencing/RNAi being inhibited by the use of conventional dsRNAs or dsRNA hairpins. There is also a risk that siRNAs of a pools can trigger silencing on non-target RNAs (“off-target effects”) e.g., via complementary base pairing of guide strands with these other RNAs which is incomplete but still sufficient to form functional RISC (Jackson and Linsley 2004; Jackson et al. 2006; Senthil-Kumar and Mysore 2011; Casacuberta et al. 2015; Kamola et al. 2015). In summary, the use of dsRNAs in which a particularly large number of siRNAs are generated which are not characterized in more detail and are not active on the target RNA is problematic (Qu et al. 2012; Dalakouras et al. 2016). The same applies to dsRNAs, from which other forms of sRNAs are generated.

Until recently, it was not possible to reliably identify and use the few siRNAs which were effective in RNAi, here referred to as “esiRNAs” or “ERNAs” and hereinafter referred to in combination as “esiRNAs/ERNAs,” from siRNA pools. Many siRNA or sRNA active agents have accordingly been designed so as to be directed against regions of a target mRNA encoding conserved motifs of a protein, or they were based on unreliable in silico predictions of a-sites in the target RNAs and then tested for their effect in very complex empirical studies (“trial and error”) (Birmingham et al. 2007; Cerritelli and Crouch 2009; Miozzi et al. 2013; Fakhr et al. 2016; Carbonell et al. 2018; Eastman et al. 2018; Han et al. 2018; Qureshi et al. 2018; Setten et al. 2019).

It is only in recent years, mainly through work from our laboratory, that it has become possible to establish an experimental screening method, referred to as the “eNA screen,” which makes it possible to reliably detect a-sites in a wide variety of target RNAs in a short period of time (Schuck et al. 2013; Gago-Zachert et al. 2019; WO2019001602 A1; WO 2022/200407; schematically depicted in FIG. 1). Knowledge of the a-sites accordingly makes it possible to identify esiRNAs/ERNAs which are reliably and efficiently effective in the RNA silencing/RNAi process against the target RNAs in question.

The terms reliable and efficient can be precisely defined: the “eNA-screen” is a multi-stage in vitro method (see further description below), in which ultimately the efficiency of hydrolysis of the target RNA by an identified siRNA is measured in a “slicer” (cleavage) assay. According to the invention, siRNAs which hydrolyze at least 25% of the quantity of target RNA used in a standardized and stringent slicer assay are denoted as efficiently effective, and thus as esiRNAs/ERNAs (see also tables 1, 2, 7 and 8). It was possible to demonstrate that these esiRNAs/ERNAs reliably demonstrate a high degree of antipathogenic efficacy in the respective in vivo system, which in the majority of cases correlates with said ability to hydrolyze the target RNA in the slicer assay (Gago-Zachert et al. 2019; see, for example, FIG. 2-6).

With the possibility of being able to exclusively use esiRNAs/ERNAs against target RNAs and thus against pathogens, the efficiency of RNA silencing/RNAi processes is significantly increased. In addition, the use of esiRNAs/ERNAs increases the specificity of RNAi: the probability of off-target effects on unaddressed RNA molecules and thus unfavorable and undesirable side effects of RNA active agents are significantly reduced.

As already explained, esiRNAs/ERNAs can be defined by the identification of a-sites in target RNAs. In other words, sequences, target-sites, are accessible in the a-sites, via which the esiRNA/ERNA guide strands can bind to the target RNA (hybridize via complementary base pairing) and, by the activity of RISC, the target RNA is then deactivated in the described way (i.e., via endonucleolytic cleavage or inhibition of translation (see above)). An analogous effect can also be achieved by related sRNAs, such as miRNAs, the sequences of which can be derived from the sequences of esiRNAs/ERNAs and which can then be used in an analogous RNA silencing/RNAi process like esiRNAs/ERNAs.

It has only recently been demonstrated that the identification of a-sites in target RNAs and the definition of esiRNAs/ERNAs, the guide strands of which can bind to target sites in these a-sites, also simultaneously enables antisense deoxyribonucleic acid (DNA) oligonucleotides (ASO) to be identified which can also bind to these target-sites (WO 2022/200407). Using analogous terminology, ASOs derived from esiRNAs/ERNAs are referred to as eASOs. eASOs have homologous DNA sequences to single strands of esiRNAs/ERNAs (i.e., deoxynucleotides instead of ribonucleotides; thymidine instead of uridine) and can correspondingly also hybridize to the respective target sites in the a-sites of the target RNAs and also cause RNA silencing. Just like sRNAs, ASOs can be used transiently; they act in the “antisense method” via other mechanisms than sRNAs. In the given context, two of these mechanisms are important: firstly, the formation of a DNA: RNA heteroduplex between the single-stranded ASO and the target RNA can inhibit the translation process of RNA in the cytoplasm. Secondly, the formation of DNA: RNA heteroduplexes can cause the activation of RNase Hendonucleases (in eukaryotic cells RNAase H1 and/or RNAase H2) in the cell nucleus or cytoplasm, which then, similar to AGO/RISC, guided by the binding of the single-stranded ASO to the complementary RNA, catalyze the breakdown of this target RNA (Shen and Corey 2018; Bennett 2019; WO 2022/200407; Wdowikowska and Janicka, 2021, Crooke et al., 2021). Optimally active ASOs have a length of 12-20 nucleotides (Crooke et al., 2021). Accordingly, eASOs can be readily derived from 21, 22, 23 or 24 nt-long esiRNAs/ERNAs.

The following text is substantially limited to the description of the activity of esiRNAs/ERNAs. Analogous findings also apply to other related sRNAs such as miRNAs and eASOs, the RNA or DNA sequences of which can be derived from the sequences of identified esiRNAs/ERNAs: due to the matching sequence, the single strands of such sRNAs or eASOs can bind to the same target sites in the corresponding target RNAs and thus also be used in RNA silencing/RNAi or RNA silencing/antisense methods for pathogen control (WO 2022/200407). The esiRNAs/ERNAs and the related sRNAs and eASOs derived therefrom, which are identified by means of the “eNA screen,” are hereinafter collectively referred to as e NAs (effective Nucleic Acids).

It should also be noted here that RNA and DNA active agents can be used in a chemically modified form: chemical modifications, conjugates or structural, inter alia as “gapmers” or “mixmers,” can considerably improve the potential of siRNA- or ASO-based active agents in RNAi orantisense methods in vivo (Setten et al. 2019; Wdowikowska and Janicka, 2022; Crooke et al., 2021; see also the claims).

The knowledge of a large number of a-sites (and thus target sites) of a target RNA is particularly important in methods aimed at combating pathogens that are variable. Specifically, viruses having an RNA genome and an RNA-dependent RNA polymerase (RdRp) as the main enzyme of viral replication exhibit high plasticity (variability) and thus the potential to rapidly develop resistance to antiviral active agents. Viral RdRps have no, or only an inefficient, editing function that can correct errors in the incorporation of nucleotides into newly synthesized daughter nucleic acid strands. The mutation and evolution rate of RNA viruses during replication is accordingly high: this is referred to as “antigenic drift.” The mutation and evolution rate of viruses which have a genome divided into several segments is particularly high. When a cell is co-infected by different viruses, these segments can sort into completely new combinations—this is referred to as “reassortment.” This leads to “antigenic shifts,” significant genetic changes that can lead to the generation of viruses having completely new properties and which pose a large threat to the respective host because an effective immune response to these viruses may not exist (“viral escape”).

However, high variability can also be found in other organisms, especially those that are subject to strong selection pressure. This is the case with plant pathogens that are routinely treated with antipathogenic substances. These mutate “under these substances” and form resistant forms. This applies, for example, to nematodes and fungi that are conventionally treated with nematicides and fungicides in agriculture and horticulture.

However, by using a broad spectrum of eNAs which were identified or derived using the “eNA screen” in HIGS or SIGS, the potential to develop pathogenic forms which become resistant to an antipathogenic method (escape) should be drastically reduced in the various organisms. Broad spectrum means that two or more eNAs are used against a target RNA or that two or more eNAs are used, which are directed against different target RNAs of the target organism. The same approaches could also achieve a broad-spectrum effect against pathogen variants (which may arise, for example, during a virus epidemic). Finally, using different eNAs against various target RNAs of different pathogens makes it possible to achieve broad-spectrum protection against different pathogens.

The use of effective eNAs therefore results in the following application advantages:

(i) the combinability of various esiRNAs/ERNAs makes it possible to increase the specificity and efficiency of the RNA silencing/RNAi method as much as possible (schematically depicted in FIG. 1). The same applies to other sRNAs, the sequence of which is derived from esiRNAs/ERNAs. The same applies to eASOs, the sequence of which is derived from esiRNAs/ERNAs and which are used in the antisense method.

(ii) a new combination of antipathogenic esiRNAs/ERNAs makes it possible to quickly and specifically adapt an RNAi immune response to pathogens which can be subject to significant changes by antigenic drifts or antigenic shifts or can arise in a completely new form. The same applies to other sRNAs derived from esiRNA/ERNA. The same applies to eASOs derived from esiRNA/ERNA that can be used in the antisense method.

(iii) economical production of nucleic acid-based active agents should considerably increase acceptance of the use of RNAi or antisense methods, e.g., in plant production. SIGS approaches can be carried out more efficiently and the use of transgenic approaches can be significantly reduced.

(iv) in order to optimally enable the simultaneous use of esiRNAs/ERNAs and at the same time also simultaneously improve the usability of RNA in HIGS and SIGS methods, these should preferably be used in the form of dsRNAs. Such “edsRNAs,” as they are referred to here, should contain the nucleotide sequences of several esiRNAs/ERNAs (or other sRNAs derived therefrom) identified using “eNA screens” (schematically depicted in FIG. 1). As a general advantage, dsRNAs have a significantly reduced degradation rate (increased half-life) in HIGS and SIGS methods (Bachman et al. 2020) compared to siRNAs which have single-stranded sequence overhangs at the 3′ ends, as described, and therefore have clear application advantages.

### Problem/Object

There is the problem in plant protection, when combating highly variable pathogens, of inactivating target RNAs of these pathogens using adaptable effective nucleic acid active agents and thereby suppressing the replication of said pathogens. The objective technical problem of the invention is therefore that of protecting plants against various, variable pathogens using sustainable nucleic acid-based methods.

To solve this problem, by using the “eNA-screen,” the invention provides various nucleic acid-based active agents (eNAs) which are efficiently effective in RNAi or antisense processes against three important plant pathogens, and also provides methods for the production thereof and methods for the use thereof. As stated above, the term “efficient” is specifically defined as a common technical feature of the eNAs used.

In one aspect, the invention relates to RNAi methods using one or more esiRNAs/ERNAs or related sRNAs, or antisense methods using of one or more eASOs, the sequences of which were derived from esiRNAs/ERNAs. Active esiRNAs/ERNAs or other sRNAs or eASOs derived therefrom (collectively referred to as eNAs) should have a reliably high, effective antipathogenic efficacy and, in the combinations used, should be directed against various target RNAs or various regions (target sites) of target RNAs, in order to also be able to reliably combat highly variable pathogens.

In a further aspect, the invention relates to RNAi methods with edsRNAs, a completely new form of dsRNAs designed according to the invention, which contain nucleotide sequences of esiRNAs/ERNAs or sRNAs derived therefrom and which are processed in the course of the RNA silencing/RNAi process into said esiRNAs/ERNAs or SRNAs.

The nucleic acid-based active agents of the invention can be generated or used against representatives of various classes of variable or highly variable pathogens that infect the host organism plant. As stated, the most important variable plant pathogens include viruses, nematodes and fungi. The invention is particularly advantageous because the nucleic acid-based active agents according to the invention can be generated or used against particularly economically important representatives of these pathogens. One representative of an economically important, highly variable viral pathogen in plants is cucumber mosaic virus (CMV). One representative of an economically important, variable nematode pathogen in plants is Meloidogyne incognita (M. incognita). One representative of an economically important, variable fungal pathogen in plants is Botrytis cinerea (B. cinerea).

The solution to the problem of the invention provides a wide range of effective eNAs that can be used individually or in combination against these important pathogens as active agents in plant protection.

### “eNA Screen” Method Used

In order to identify eNAs, use was made of the published method, described in a patent application (WO 2019/001602) and referred to here as “eNA screen”: it was used for the first time in a new, standardized and stringent form on the target RNAs mentioned below of cucumber mosaic virus, Meloidogyne incognita and Botrytis cinerea, and thus led to the first possible identification of a class of effective eNAs against these pathogens in RNA silencing, i.e., esiRNAs/ERNAs and sRNAs, eASOs and edsRNAs derived therefrom (see the following description).

The eNA screen method takes place in three steps.

(i) a target RNA, which can be a genomic RNA, an mRNA or a dsRNA, is added to a cytoplasmic extract from plant cells (Nicotiana tabacum BY-2 cells), what is referred to as “BYL” (L stands for “lysate”). The (endogenous) DCLs present in the extract generate an siRNA pool from the target RNA, which pool contains, inter alia, 21, 22, 23 and 24 nt siRNAs as the main products. The siRNA pool is captured in its entirety by next-generation RNA sequencing (NGS, RNA-Seq).

(ii) in the extracts, RISC is reconstituted with the siRNA pool resulting from (i) and with an AGO protein of choice. To this end, the AGO protein is translated in vitro in the extract from an added mRNA. The AGO/RISC formed are immunoprecipitated, and the siRNA strands enriched by binding to AGO are again identified by RNA-Seq. The RNA Seq data from step (i) are used here for comparison to define an enrichment in the relevant AGO/RISC.

(iii) finally, from the siRNAs captured in (ii), using further in vitro assays in which testing for endonucleolytic cleavage (slicing) with the respectively used AGO/RISC is carried out (referred to hereinafter and in the figures as “slicer assay”), those siRNAs which were previously identified as being enriched in the relevant AGO/RISC and which induce efficient slicing of the target RNA are identified. As already mentioned, siRNAs are defined as esiRNAs/ERNAs if, in a standardized and stringent in vitro slicer assay with the corresponding AGO/RISC, they induce the hydrolysis of 25% or more of the quantity of target RNA used in the assay in question.

Further validation steps then test the efficacy of the respective esiRNAs/ERNAs in vivo. Different methods adapted to the respectively addressed target pathogens are used here. Other sRNAs and eASOs constructed with an analogous nucleotide sequence are derived from esiRNAs/ERNAs identified in this way. These can also be tested in BYL in slicer assays. The active components in eASOs are RNAase H enzymes (see, for example, WO 2022/200407).

### Addressed Target Organisms

### The Plant Pathogenic Cucumber Mosaic Virus (CMV)

CMV, the type-determining virus of the genus Cucumovirus (Bromoviridae family), is a plant pathogen of great economic importance (Scholthof et al., 2011; Rybicki, 2015; Gallitelli, 2000). CMV has a tripartite-segmented, single-stranded (ss) positive (+)-strand RNA genome. The three genomic RNAs 1 (3.3 kb), 2 (3.0 kb) and 3 (2.2 kb), which are packaged in three different virus capsids and only trigger an infection together, have a cap at the 5′ end and a tRNA-like structure at the 3′ end. Due to the segmentation of the genome, CMV is a reassorting virus with the associated high mutation rate: in other words, in addition to antigenic drift caused by the viral RdRP, the virus exhibits antigenic shifts. After entering the host cell, the three genomic RNAs act as mRNAs due to their (+) orientation ((+) is sense orientation, like an mRNA). RNA 1 and RNA 2 encode proteins “1a” (111 kDa) and “2a” (97 kDa), respectively. The 2a protein is the RdRp; together with 1a, it forms the viral component of the replicase, which catalyzes genome replication and transcription of subgenomic (sg) RNAs.

During the RNA replication process (not described in detail here), complementary (−)RNA copies of the viral RNAs are transcribed by the replicase. These then serve both as templates for the synthesis of new (+)RNA molecules and as templates for the synthesis of subgenomic sgRNAs. An sgRNA, sgRNA 4 (1.1 kb), is transcribed from the (−)RNA copy of RNA 3 produced during replication and is also packaged together with it into a capsid. RNA 3 itself encodes the 30 kDa “movement protein 3a”; the sgRNA 4 for the 24 kDa “capsid protein CP.” 3a and CP are essential for both cell-to-cell and systemic movement of the virus through the plant during the infection process. Another subgenomic RNA, sgRNA 4A (0.7 kb), is transcribed from the (−)RNA copy of RNA 2. This encodes a viral suppressor of RNA silencing (VSR), “2b” (15 kDa). 2b interferes with the RNA silencing/RNAi process, inter alia by the sequestering (high-affinity binding) of siRNAs generated in the process. All five gene products influence the spread of the virus in the plant, and hence also virulence, in a host-specific manner. During infections, additional satellite RNAs can be produced during viral RNA replication, the presence of which can considerably influence pathogenesis (Gallitelli, 2000; Garcia-Arenal et al., 2008; Jaquemond, 2012; Roossinck et al., 2001; Roossinck et al., 2002, Palukaitis, 2016; Nouri et al., 2014; Mochizuki and Ohki 2012).

CMV strains are roughly divided into two subgroups, 1 (I) and 2 (II), with the strains of subgroup I being further subdivided into two (A and B) subgroups (see also below and FIG. 7). While sequence similarities are high within a subgroup (subgroup I: >88%; subgroup II: >96%), they are only 70-75% between the subgroups. CMV strains of subgroups IA and II are present around the world; those of subgroup IB are mainly found in eastern Asia (Garcia-Arenal et al., 2008; Jaquemond, 2012; Nouri et al., 2014; Mochizuki and Ohki 2012). The serological differentiation index (SDI) between the CMV subgroups is approximately 1-2. These differ from the other cucumoviruses PSV (peanut stunt virus) and TAV (tomato aspermy virus) with an SDI of 6-7. Like other segmented RNA viruses, CMV has high variability. This is caused by the accumulation of mutations by antigenic drift and shift processes (Scholthof et al., 2011; Rybicki, 2015; Gallitelli, 2000).

In contrast to other members of the Bromoviridae family, CMV strains have a very broad collective host range and infect more than 1200 plant species in over 100 families of monocotyledons and eudicotyledons/dicotyledons. These include important fruit, vegetable and ornamental plants such as Fabaceae, Cucurbitaceae, Convolvulaceae and Solanaceae. CMV is thus the plant virus that has the broadest host range and infects important crops such as beans, beets, carrots, celery, lettuce, peppers, melons, pumpkins, tomatoes and spinach (Scholthof et al., 2011; Garcia-Arenal et al., 2008; Jaquemond, 2012; Mochizuki and Ohki 2012). CMV infections cause, inter alia, severe systemic mosaic symptoms, leaf deformation, systemic necrosis, chlorosis, dwarf growth, and fruit lesions (Garcia-Arenal et al., 2008; Jaquemond, 2012; Holeva et al., 2021). CMV can interact synergistically with potyviruses, tobamoviruses and potato virus X (PVX) in the Solanaceae, as well as with potyviruses in Cucurbitaceae hosts (Scholthof et al., 2011; Gallitelli, 2000; Jaquemond, 2012).

CMV particles are transmitted by more than 80 species of aphids across 33 genera by the non-persistent “stylet-borne” mode. In addition, depending on the plant species, transmission can occur through infected seeds (Jaquemond, 2012; Ali and Kobayashi, 2010; O'Keefe et al., 2007). There are also indications that the virus can survive the winter months in seeds, and this then becomes a significant source of primary infections at the beginning of the growing season.

Due to its wide range of host plants, its presence around the world and its non-persistent transmissibility by numerous aphids, CMV is counted among the plant viruses of greatest economic importance and is classed as one of the most important viruses in annual crops around the world (Scholthof et al., 2011; Rybicki, 2015). Crop losses vary from year to year at different locations and are difficult to quantify, in particular in cases of mixed infections. Estimates from the 1990s and 2000s provide some insight; for example in China they assume a tomato harvest of 25%-50%, or in Spain a melon harvest or a pepper harvest of 60% or 80%, respectively. When necrogenic satellite RNA arose with certain CMV strains, losses of 80%-100% of tomato plants were recorded in 70% of the growing areas in Spain and Italy (Gallitelli, 2000). Remarkably, more CMV hosts and new CMV-induced plant diseases are described every year. It is anticipated that increased aphid activity in the northern temperate regions due to climate change will lead to further epidemics (Scholthof et al., 2011; Nicaise, 2014). However, CMV is also becoming increasingly important in tropical and subtropical regions, in particular where mixed crops are grown.

Control measures based solely on the use of pesticides against aphids are not very effective (Gallitelli, 2000). However, approaches using transgenic plants or RNA-based approaches for plant protection have also not been very successful to date, mostly due to the high variability of the virus (Nicaise, 2014).

### SUMMARY OF THE INVENTION

To solve the problem, it was possible in the scope of the invention, by means of the eRNA screen method, to characterize esiRNAs/ERNAs against various genomic CMV RNAs which reliably have high antiviral efficacy. In addition to a generally high antiviral efficacy against the cognate target RNAs, these esiRNAs/ERNAs or variants of these esiRNAs/ERNAs should also be antivirally effective against various CMV variants. In addition, dsRNAs, referred to as “edsRNAs,” which are preferably used for HIGS and/or SIGS methods were generated, from which, in the course of the RNA silencing/RNAi process, significant quantities of esiRNAs/ERNAs are generated and which therefore also have a reliable and efficient antiviral effect. From the esiRNAs/ERNAs characterized in this way, it was also possible to derive other sRNAs or eASOs which can also be used in RNA silencing/RNAi methods or in RNA silencinglantisense methods against CMV.

The Plant Pathogenic Nematode Meloidogyne incognita

In evolutionary terms, nematodes are the oldest multicellular worms. In the numerous moist habitats in which they are present, they often represent the largest group in the metazoan fauna, both in terms of the number of individuals and species diversity (Wikipedia). They have a very simple structure: their body is limbless, cylindrically elongated and smooth, and surrounded by an elastic cuticle. The cuticle is secreted by an epidermal cell layer and forms the nematode exoskeleton (Bird and Bird, 1991a). The cuticle is permeable to ions and water and regulates the hydrostatic pressure of the nematode body. Most nematodes undergo four molts during their development from the juvenile stage (stages J1 to J4) until they reach the adult stage. In the process, the cuticle is either completely shed or, as in the case of Meloidogyne, partially absorbed (Perry and Moens, 2011). Beneath the epidermis, muscles are aligned longitudinally along the inside of the body and are activated by two nerves on the dorsal and ventral sides, also running longitudinally and connected by nerve rings (Bird and Bird, 1991b). The worm's head has some sensory organs and a “mouth” that opens into a muscular pharynx. The latter serves as a pump to draw food into the adjoining intestine. The intestine opens into a long, simple muscleless intestinal cavity and finally an anus near the tip of the body. There is no vascular system for the distribution of digested food and no respiratory system for the absorption or distribution of oxygen. Instead, nutrients and waste are distributed in the pseudo-celomic body cavity, the contents of which are regulated by an excretory canal along each side of the body (Bird and Bird, 1991c).

One subgroup of nematodes is the plant-parasitic nematodes (PPN). PPNs infect many plant species and cause enormous crop losses around the world (Blok et al., 2008). Despite very different lifestyles and feeding strategies, all PPNs have a hollow, protruding stylet that serves to pierce plant cell walls and to inject secretions and/or enzymatically active proteins that facilitate infection and the uptake of nutrients. The secretions or proteins are produced by three esophageal “salivary glands,” the cuticle and chemoreceptor organs (Perry, 1996; Semblat et al., 2001; Curtis, 2007).

Among the PPNs, what are referred to as “sedentary endoparasites” cause the greatest economic damage. These include the root knot nematodes (RKNs), among which the Meloidogyne species, and in this case Meloidogyne incognita, are the most important plant pathogens (Trudgill and Blok, 2001), causing estimated crop losses of approximately 10 billion euros per year around the world. RKNs are found particularly in temperate and tropical regions of the world (Blok et al., 2008; Abad and Williamson, 2010). They infect thousands of plant species, including virtually all crops, and they cause typical root deformations (galls), which lead to weak and low-yielding plants.

The life cycle of RKNs extends over 3-10 weeks, depending on the nematode species and environmental conditions. The worm-shaped juveniles of the second stage (J2) hatch from the eggs into the soil to infect the roots of the host. In the preparasitic phase, the J2 usually penetrate the root tissue behind the root tips by physically piercing the root cells with their stylet. At the same time, they release cell wall-altering enzymes that enable further migration into the root tissue and between the cells to the root tip. From there, they migrate into the vascular cylinder of the plant (Perry and Moens, 2011) and induce the formation of specialized feeding sites in the form of “giant cells” (GCs). GCs are hypertrophied and multinucleate. They are formed by repeated nuclear divisions and cell growth in the absence of cell division (Jones and Payne, 1978; Caillaud et al., 2008) and are the only source of nutrients for the nematodes. The nematode pierces the cells to gain access to the cytoplasm, while at the same time secretions change the hydrostatic pressure of the cells so that easy uptake of nutrients is possible (Abad and Williamson, 2010). The divisions of the vascular cells and feeding cells surrounding the nematode lead to the formation of a typical gall. After establishing the feeding sites, the J2 become sedentary and then grow into adult females or males through three further molts (parasitic J3 and J4). The females are sedentary, while the males become mobile again and migrate from the root into the soil. RKNs reproduce by mitotic parthenogenesis (Castagnone-Sereno et al., 2013); sex is determined by environmental conditions: under poor nutritional conditions, the number of males increases (Papadopoulou and Triantaphyllou, 1982). At the end of their development, the female RKNs become pear-shaped. They produce hundreds to thousands of eggs in a protective, gelatinous matrix on the outer surface of the root, which are released directly into the rhizosphere. In the egg, the first-stage juveniles molt after embryogenesis to J2, and then hatch to continue the life cycle under favorable conditions in a suitable host (Hussey and Mims, 1991; Chitwood and Perry, 2009; Curtis et al., 2009).

Various chemicals such as methyl bromides and carbamates were used as nematicides for a long time. However, most are now prohibited due to the potential environmental and health risks for consumers associated with their use. An alternative method for PPN control is crop rotation, in which nematode-resistant plants are grown one after the other in different seasons. However, this practice has limitations: some nematodes such as Meloidogyne have such a broad host range that it is difficult to select suitable crops to interrupt the infestation cycle. Biological control of PPNs involves the use of one or more organisms such as species of nematophagous fungi or bacteria. These are introduced into the soil where they attack the nematodes without affecting plant growth (Evans et al., 1993). However, such “nematode predators” are difficult to manage on a large scale. In addition, environmental factors such as soil condition, moisture, temperature and pH significantly influence the survival of biocontrol agents (Chen and Dickson, 2004; Stirling, 2014), which does not facilitate their use. For nematodes such as Caenorhabditis elegans (C. elegans), but also Meloidogyne incognita (M. incognita), it is described that they take up siRNAs and also dsRNAs via the above-described nutrient uptake mechanism, and that siRNAs can then also become active in the animal's body in the worm's RNA silencing/RNAi system by uptake into cells, e.g., active in a nematicidal manner, by inactivating mRNAs of essential proteins (Arguel et al., 2012; Bakhetia et al., 2005; Banakar et al., 2020; Chaudhary et al., 2019; Dalzell et al., 2010a; Dalzell et al., 2010b; Danchin et al., 2013; Dong et al., 2014; Dong et al., 2016; Dutta et al., 2015; Huang et al., 2006; Iqbal et al., 2020; Niu et al., 2012; Papolu et al., 2013; Shivakumara et al., 2016).

Analogously to that described above for CMV, esiRNAs/ERNAs and edsRNAs have been characterized that reliably have high nematicidal efficacy against various variants of M. incognita. From the esiRNAs/ERNAs characterized in this way, it was also possible to derive other sRNAs or eASOs which can also be used in RNA silencing/RNAi methods or in RNA silencinglantisense methods against M. incognita.

The Plant Pathogenic Fungus Botrytis cinerea

Botrytis cinerea (teleomorph: Botryotinia fuckeliana), the main cause of gray mold disease, is an aggressive necrotrophic fungal pathogen that can infect a large number of plant species (more than 200 species) (Elad, 1997; van Kan, 2006; Choquer et al., 2007; Williamson et al, 2007; Nakajima & Akutsu, 2014). B. cinera secretes nonspecific phytotoxins that kill the cells of a broad spectrum of plants (Pinedo et al., 2008). The economic damage caused, 10-100 billion US dollars per annum worldwide, with up to 40% losses in greenhouse and field crops if no chemical control agents are used, make B. cinerea one of the 10 most important plant fungal pathogens (Dean et al., 2012; Pedras et al., 2011; Villa Rojas et al., 2012). The use of chemical fungicides leads to significant environmental damage (Malhat et al., 2015; Oliveira et al., 2015; Tomenson and Matthews, 2009) and is limited by the nutritional versatility of B. cinerea. In addition, several cases of fungicide resistance in B. cinerea have been reported (Leroux, 2007).

Taxonomically, B. cinerea belongs to the Ascomycota phylum in the Leotiomycetes class and the Sclerotiniaceae family (Garfinkel, 2021). Two groups, I and II, have been proposed as phylogenetic species. The strains of group I (also referred to as “Botrytis pseudocinerea”) and Group II (“B. cinerea sensu stricto”) differ in their ecology and fungicide resistance pattern (Fournier et al., 2003, 2005). The genome of two strains (B05.10 and T4) has been completely sequenced (Choquer et al., 2007).

In contrast to many other plant pathogens, B. cinerea is present year round and under a wide range of environmental conditions (Nair et al., 1995). The infection/nutrition process of B. cinerea is usually described by the following phases: conidia produced on sclerotia of infected plants or plant debris attach to the surface of a new host and form a germ tube. This develops into an appressorium that facilitates penetration into the host surface. To overcome the host cuticle barrier, B. cinerea also secretes cell wall degrading enzymes (CWDEs). Before the infection hyphae penetrate, epidermal cells and mesophyll cells die. A number of metabolites and proteins secreted by the fungus induce symptoms of programmed cell death (PCD) or have been shown to cause cell death (Choquer et al., 2007; Nakajima and Akutsu, 2014).

HIGS has already been used to control B. cinerea. The basis for evidence of pathogenicity or virulence genes was provided by knockout mutants (Nakajima and Akutsu, 2014). Thus, it was also possible to determine, inter alia, potential target genes, some of which are used in the application example described in more detail below (ten Have et al., 1998; Li et al., 2019; Liu et al., 2018; Nerva et al., 2020; Qiao et al., 2021; Schumacher et al., 2008; Segmüller et al., 2008; Soulie' et al., 2006; Ren et al., 2018; Yang et al., 2013; Zheng et al., 2000). In addition to HIGS, topical/transient applications of dsRNA have also already been considered as an alternative approach (Wang et al., 2016; Weiberg et al., 2013), and some studies have also shown a certain effect in various fungi (McLoughlin et al., 2018; Gebremichael et al., 2021). The current state of scientific knowledge is that the success rate of this method is heavily dependent on the potential of the fungus to take up external RNA (Qiao et al., 2021).

Analogously to that described above for CMV and M. incognita, esiRNAs/ERNAs and edsRNAs have been characterized that reliably have high fungicidal efficacy against various variants of B. cinerea. From the esiRNAs/ERNAs characterized in this way, it was also possible to derive other sRNAs or eASOs which can also be used in RNA silencing/RNAi methods or in RNA silencinglantisense methods against M. incognita.

### DETAILED DESCRIPTION OF THE INVENTION

The problem of the invention is solved by a nucleic acid for the protection of plants against the plant pathogens cucumber mosaic virus, Meloidogyne incognita and Botrytis cinerea, wherein
a. the nucleic acid is a small interfering RNA (siRNA) composed of completely or partially complementary nucleic acids that consists of 21, 22, 23 or 24 base pairs and contains two single-stranded RNAs selected from a guide strand and a passenger strand, wherein the guide strand and the passenger strand are selected from the group consisting of the nucleic acids having SEQ ID NOs: 1-4, 6-11, 14-17, 21-25, 27-30, 32-37, 40-43, 47-51, 53, 55-66, 69-70, 73, 75-86, 89-90, 93-120, 124, 126-132, 134-138, 140-150, 154, 156-162, 164-168 and 170-180; or
b. the nucleic acid is an siRNA according to group a., wherein at least one of the single-stranded RNAs selected from a guide strand and a passenger strand has modifications at 1 to 7 positions in the nucleotide sequence; or
c. the nucleic acid is a small RNA (sRNA) selected from an siRNA and a micro RNA (miRNA), wherein the RNA double strand thereof consists of complementary or partially complementary nucleic acids of group a. and/or group b.; or
d. the nucleic acid is a double-stranded RNA containing nucleotide sequences of at least two siRNAs or sRNAs of groups a., b., or c.; or
e. the nucleic acid is a single-stranded DNA that consists of 12 to 25 nucleotides and contains a sequence of 12 or more nucleotides which are homologous (deoxyribonucleotide instead of ribonucleotide) to one of the nucleotide sequences of the single-stranded RNAs, selected from a guide strand and a passenger strand, of groups a. or b.; or
f. the nucleic acid is a single-stranded DNA according to group e. which has modifications at 1 to 7 positions in the nucleotide sequence;
wherein the nucleic acid is provided for protection against plant pathogens using a method for the targeted identification of effective small interfering RNAs (esiRNAs/ERNAs) and sRNAs derived therefrom, and also eASO (effective antisense DNA oligonucleotides) of different lengths, collectively referred to as effective Nucleic Acids (eNAs), comprising the steps of
   (i) an RNA which was selected as a target for RNA silencing (RNAi) is produced using in vitro transcription and converted to small interfering RNAs (siRNAs) in cytoplasmic extracts of plant cells by means of endogenous Dicer-like proteins (DCL);
   (ii) a DCL-generated siRNA pool being formed from the RNA used and the siRNAs contained in this pool being determined by means of RNA-seq analysis;
   (iii) a messenger RNA (mRNA) of an argonaute (AGO) protein, which mRNA was synthesized by in vitro transcription, is added to the cytoplasmic plant cell extract, the mRNA being constructed such that it encodes the AGO protein in question with a tag;
   (iv) AGO protein molecules are formed using in vitro translation, said AGO protein molecules forming RNA-induced silencing complexes (RISC) with the DCL-generated siRNAs that are present;
   (v) tagged siRNA-loaded AGO/RISC are immunoprecipitated from BYL and the bound siRNA guide strands are determined by means of RNA-seq analysis;
   (vi) those siRNAs that are enriched in AGO/RISC are determined using the comparison of the RNA-seq data with the RNA-seq data from step (ii);
   (vii) subsequently produced synthetically and tested for their functionality in a Slicer assay with labeled target RNA, and
   (viii) in this way, esiRNAs/ERNAs are identified, and sRNAs, derived therefrom, as well as eASO, collectively referred to as eNAs, are determined;
   characterized in that
      I. in order to form the RISC in step (vii), 0.5 μmol of the mRNA of the AGO protein to be used is translated in a reaction solution containing 50% (v/v) BYL, in the presence of 10-100 nM of the synthetic siRNA to be characterized and a 10-fold excess (0.1-1 μM) of a competitor siRNA (e.g., siR gf698: guide-und passenger-strand selected from the group consisting of SEQ ID NOs: 205, 206, 207, and 208; Iki et al., 2010);
      II. after an incubation time of 2.5 h at 25° C. per batch in step I., 3.4 μmol of a nonspecific mRNA (e.g., encoding the Firefly luciferase protein, SEQ ID NO: 209; Schuck et al., 2013) is added as a further competitor RNA, and also 10 fmol of the target RNA is added, the target RNA being labeled, and the reaction batches are incubated again for 15 min at 25° C.;
      III. during the incubation in step II., the target RNA is cleaved by the AGO/RISC formed;
      IV. after gel electrophoresis of the extracted RNA, the remaining quantity of target RNA or the resulting cleavage products compared to a control reaction (carried out without siRNA) is quantified by measuring the band intensities (ImageQuantTL or ImageJ);
      V. classification as esiRNAs/ERNAs takes place using the cleavage activity (slicer activity) on the target RNA, measured in this way, of each RISC formed with this siRNA,
      VI. esiRNAs/ERNAs which endonucleolytically convert at least 25% or more of the quantity of target RNA originally used in method step II. to cleavage products are selected; and
      VII. optionally other sRNAs and eASO are derived from the sequence of esiRNAs/ERNAs identified in this way.

In step I, BYL preferably has a defined protein quantity and translation activity: the protein quantity is determined using a conventional Bradford assay (Wikipedia) and should be 7-12 mg/ml extract (Gursinsky et al., 2009). The translation activity is determined under translation conditions with 85 fmol of firefly luciferase mRNA: the measurable activity of the luciferase translated under these conditions should be in the range of at least 106 RLU (relative light units) of a converted substrate (e.g., luciferol; Wikipedia) (Gursinsky et al., 2009; Schuck et al., 2013; Gago-Zachert et al., 2019).

The quantity used of the siRNA to be tested in step I is preferably adjusted to the activity of the respective AGO protein with the siRNA siR gf698 (guide and passenger strand selected from the group consisting of SEQ ID NOs: 205, 206, 207, and 208) on the mRNA target thereof, encoding GFP (green fluorescent protein): the measurable cleavage activity with siR gf698 and the GFP mRNA target in the slicer assay is usually 90%: in other words, 90% of the quantity used of target RNA is converted to cleavage products.

In step II, the target RNA can be labeled by any means known to a person skilled in the art. For example, the target RNA may have a fluorescent marker or be radiolabeled. Preferably, the target RNA in step II. is radiolabeled.

esiRNAs/ERNAs that have a cleavage efficiency of at least 25% in vitro (step VI) exhibit a clearly measurable antipathogenic effect in vivo compared to control siRNAs (see exemplary embodiments below and tables 1, 2 and 7). The invention thus advantageously provides eNAs (effective Nucleic Acids) which can be used in RNA silencing/RNAi or RNA silencing/antisense methods as active agents against various variably plant pathogens.

esiRNAs/ERNAs that have a cleavage efficiency of at least 25% or more in vitro are, for example, siRNAs of group a. that consist of 21, 22, 23 or 24 nucleotides and contain two nucleic acids selected from a guide strand and a passenger strand which are selected from the group consisting of nucleic acids having SEQ ID NOs: 1-4, 6-11, 14-17, 21-25, 27-30, 32-37, 40-43, 47-51, 53, 55-66, 69-70, 73, 75-86, 89-90, 93-120, 124, 126-132, 134-138, 140-150, 154, 156-162, 164-168 and 170-180.

esiRNAs/ERNAs that have a cleavage efficiency of at least 50% or more in vitro are preferred according to the invention. esiRNAs/ERNAs that have a cleavage efficiency of at least 50% or more in vitro are, for example siRNAs of group a. that consist of 21, 22, 23 or 24 nucleotides and contain two nucleic acids selected from a guide strand and a passenger strand which are selected from the group consisting of nucleic acids having SEQ ID NOs: 1, 2, 4, 6-11, 14-17, 21-25, 27, 28, 30, 32-37, 40-42, 47-51, 55-66, 75-86, 93-120, 128-132, 134, 135, 138, 140-150, 158-162, 164, 165, 168, and 170-180.

esiRNAs/ERNAs that have a cleavage efficiency of at least 75% or more in vitro are more preferred according to the invention. esiRNAs/ERNAs that have a cleavage efficiency of at least 75% or more in vitro are, for example siRNAs of group a. that consist of 21, 22, 23 or 24 nucleotides and contain two nucleic acids selected from a guide strand and a passenger strand which are selected from the group consisting of nucleic acids having SEQ ID NOs: 2, 4, 6, 8, 10, 11, 14-16, 21, 22, 24, 25, 28, 30, 32, 34, 36, 37, 40-42, 47, 48, 50, 51, 55, 60-63, 65, 69, 75, 80-83, 85, 89, 94-101, 103-106, 108-115, 117-120, 130, 134, 140, 143, 145, 146, 160, 164, 170, 173, 175, and 176.

esiRNAs/ERNAs that have a cleavage efficiency of at least 90% or more in vitro are particularly preferred according to the invention. esiRNAs/ERNAs that have a cleavage efficiency of at least 90% or more in vitro are, for example siRNAs of group a. that consist of 21, 22, 23 or 24 nucleotides and contain two nucleic acids selected from a guide strand and a passenger strand which are selected from the group consisting of nucleic acids having SEQ ID NOs: 4, 6, 10, 11, 14-16, 21, 22, 24, 30, 32, 37, 40-42, 47, 48, 50, 61, 81, 96, 97, 99, 101, 104-106, 110, 111, 113, 115, and 118-120.

esiRNAs/ERNAs that have a cleavage efficiency of at least 95% or more in vitro are particularly preferred according to the invention. esiRNAs/ERNAs that have a cleavage efficiency of at least 95% or more in vitro are, for example, siRNAs of group a. that consist of 21, 22, 23 or 24 nucleotides and contain two nucleic acids selected from a guide strand and a passenger strand which are selected from the group consisting of nucleic acids having SEQ ID NOs: 6, 10, 32, 36, 97 and 11.

The sequences of the guide strands and the associated passenger strands are each given in tables 1, 2, 7 and 8.

The method steps (i) to (vii) are described in WO2019001602 A1. WO2019001602 A1 is incorporated herein by reference.

The method steps I. to VII. are new and are based on the exemplary embodiments of the present application.

According to group a., the nucleic acid is an siRNA that consists of 21, 22, 23 or 24 nucleotides and contains two nucleic acids selected from a guide strand and a passenger strand, wherein the guide strand and the passenger strand are selected from the group consisting of the nucleic acids having SEQ ID NOs: 1-4, 6-11, 14-17, 21-25, 27-30, 32-37, 40-43, 47-51, 53, 55-66, 69-70, 73, 75-86, 89-90, 93-120, 124, 126-132, 134-138, 140-150, 154, 156-162, 164-168 and 170-180. The single-stranded nucleic acids having SEQ ID NOs: 1-4, 6-11, 14-17, 21-26, 27-30, 32-37, 40-43, 47-51, 53, 55-66, 69-70, 73, 75-86, 89-90, 93-120, 124, 126-132, 134-138, 140-150, 154, 156-162, 164-168 and 170-180 are components (single-stranded components such as guide strands and passenger strands) of the effective nucleic acids, newly identified in the scope of the invention, which can be used in the form of small RNAs, sRNAs, in RNA silencing/RNAi plant protection methods as active agents against various variable plant pathogens.

According to group b., the nucleic acid of the invention is an siRNA comprising two single-stranded RNAs selected from a guide strand and a passenger strand, according to group a., wherein the guide strand and/or the passenger strand have modifications at 1 to 7 positions in the nucleotide sequence. The nucleic acids of group b, thus represent variants of the nucleic acids of group a. Within the meaning of the invention, “modification” means “base substitution,” i.e., that a base selected from adenine (A), uracil (U), guanine (G) and cytosine (C) can be substituted by any of the other bases. Such base substitutions can occur at 1 to 7 positions in the nucleotide sequence of a nucleic acid of group a. The base substitutions are preferably independent of each other at each position, i.e., each of the 1 to 7 positions in the nucleotide sequence of a nucleic acid of group a. can be substituted independently of each other by any of the other bases.

According to group c., the nucleic acid of the invention is a small RNA (sRNA, such as a small interfering RNA, siRNA, or such as a micro RNA, miRNA), the RNA double strand of which consists of completely complementary or partially complementary nucleic acids of group a. and/or group b.

Small interfering RNAs, abbreviated to siRNAs, are short, double-stranded, noncoding ribonucleic acid molecules of 20 to 25 base pairs in length which are phosphorylated at the 5′ end and have a 2 nucleotide (nt)-long, single-stranded overhang at the 3′ end. As described above, siRNAs are generated under natural conditions in a cell from double-stranded regions of RNA molecules, usually non-self RNA; however, they can also be used in a targeted manner in synthetic form in RNA silencing/RNAi methods. Except for the mentioned 3′ overhang, the two constituent single strands of an siRNA are completely (i.e., over the entire length of the RNA) base-paired via complementary base pairing (usually adenine (A) with uracil (U) and guanine (G) with cytosine (C)) and form an RNA duplex. During the RNA silencing/RNAi process, one of the two single-stranded components of the siRNA associates with complementary single-stranded regions (target sites) of other ribonucleic acid molecules, referred to here as target RNAs. These are predominantly the cognate RNAs from which the siRNAs were originally generated. The function of the target RNAs can thus be inhibited or modulated in various ways. In eukaryotes, for example, siRNAs can be used to suppress the replication of pathogens such as viruses or the expression of cellular genes at the post-transcriptional level.

Micro RNAs, abbreviated to miRNAs, are short, noncoding ribonucleic acids that are encoded by the cellular genome in the form of precursor molecules. After transcription and processing, complementary regions of the miRNA, which generally consists of 21 to 23 nt-long RNA strands, form double strands via base pairing. In contrast to siRNAs, these double-stranded regions in miRNAs can be interrupted by single-stranded regions in the form of “mismatches” (relating to individual nucleotides) and/or “loops” and/or “bulges” (relating to several nucleotides). Like siRNAs, miRNAs inactivate or modulate by binding (hybridization) to completely or incompletely complementary regions of a target RNA, the function of which is determined by the RNA silencing/RNAi process. Accordingly, miRNAs play a central role in the post-transcriptional regulation of cellular gene expression and, like siRNAs, can also be used to artificially modulate cellular gene expression.

As already explained above, two nucleic acid strands are referred to as “complementary” if their nucleotides can base pair with each other. The pairing rule for DNA was originally defined according to the conventional pairing rule of nucleotide bases according to Watson and Crick, where, by means of hydrogen bonding, adenine (A) from one nucleic acid strand can interact (base pair) with thymine (T) from the other nucleic acid strand and guanine (G) from one nucleic acid strand can interact (base pair) with cytosine (C) from the other nucleic acid strand. As a result, DNA forms a double strand, with each strand being, in a manner of speaking, the negative version of the other strand.

RNA molecules can form double strands according to a similar pattern, i.e., via hydrogen bonding of adenine (A) with uracil (U) (RNA molecules do not contain thymine, but rather uracil) and of guanine (G) with cytosine (C), but also via other base pairings not described here. These double strands can be formed intermolecularly, i.e., between different RNA molecules, or intramolecularly, i.e., within one RNA molecule. As explained above, double-stranded regions of an RNA can comprise many nucleotide building blocks (potentially hundreds or thousands) and thus a corresponding number of base pairs. In this case, as explained above, the term “dsRNA” is used. Double strands formed intramolecularly are an essential determinant of the formation of complex structures within an RNA. It is also possible for complementary RNA and DNA molecules to form double strands, which are referred to as DNA: RNA hybrids or DNA: RNA heteroduplexes.

“Completely complementary” usually defines two nucleic acid strands that can form base pairs over their entire length and over their total number of nucleotide building blocks, and thus can form a double strand. As stated, functional siRNAs and other sRNAs have overhangs of one or more nucleotides at the termini. Likewise, dsRNAs can have overhangs of one or more nucleotides at the termini, or can have spacers (see definition above). Within the meaning of the invention, the term “completely complementary” refers here accordingly to the nucleotide sequences of the nucleic acids of groups a., b., c. and d., excluding these overhangs or spacers.

Within the meaning of the invention, “partially complementary” accordingly defines nucleic acid strands that are not completely complementary and therefore cannot form base pairings over the entire length and total number of nucleotide building blocks. Double-stranded regions of these nucleic acids are interrupted by single-stranded regions such as mismatches, loops and/or bulges.

It is particularly preferred in group c. when the nucleic acid is an siRNA, the RNA double strand (duplex) of which consists of completely complementary nucleic acids of group a. and/or group b. In the scope of the invention, these are siRNAs that are newly identified by means of “eNA screens” and, according to the above definition, in corresponding AGO/RISC, induce the hydrolysis of 25% or more, preferably 50% or more, more preferably 75% or more, particularly preferably 90% or more, particularly preferably 95% or more of the respectively used amount of target RNA used in a standardized and stringent in vitro slicer assay, and have therefore been classified as esiRNAs/ERNAs (effective siRNAs) and can be used in RNA silencing/RNAi plant protection methods as active agents against various variable plant pathogens.

It is also preferred in group c. when the nucleic acid is an sRNA, for example an miRNA, the RNA double strand of which consists of partially complementary nucleic acids of group a. and/or group b. These are sRNAs, the nucleotide sequence of which can be derived from the abovementioned esiRNAs/ERNAs and which can also be used in RNA silencing/RNAi plant protection methods as active agents against variable plant pathogens.

According to group d., the nucleic acid of the invention is a double-stranded RNA containing nucleotide sequences of at least two siRNAs or sRNAs of group c. In the scope of the invention, these are entirely newly designed and engineered double-stranded ribonucleic acids (edsRNAs; effective double-stranded RNAs) that contain the nucleotide sequences of identified esiRNAs/ERNAs or other sRNAs derived therefrom, such as miRNAs, and that can be used in RNA silencing/RNAi plant protection methods as active agents against variable plant pathogens.

Within the meaning of the invention, this means that the RNA double strand of these nucleic acids contains nucleotide sequences of at least two nucleic acids of groups a., b. or c.

In a preferred embodiment, a nucleic acid of group d. contains the nucleotide sequences of at least two small RNAs (sRNA, such as a small interfering RNA, siRNA, or such as a micro RNA, miRNA), the RNA double strand of which consists of completely or partially complementary nucleic acids of group a. and/or group b. In a further preferred embodiment, a nucleic acid of group d. has the nucleotide sequences of at least two siRNAs, the RNA double strand of which consists of completely complementary nucleic acids of group a. and/or group b. In a further preferred embodiment, a nucleic acid of group d. contains the nucleotide sequences of at least two sRNAs, such as miRNAs, the RNA double strand of which consists of partially complementary nucleic acids of group a. and/or group b.

In a further preferred embodiment, a nucleic acid of group d. contains the nucleotide sequences of two, three, four, five . . . to an infinite number of siRNAs and/or sRNAs of groups a, b, or c. Preferably, a nucleic acid of group d. contains the nucleotide sequences of 2 to 100 siRNAs and/or sRNAs of groups a, b, or c. It is particularly preferred if the nucleic acid of group d. contains the nucleotide sequences of 2 to 90, 2 to 80, 2 to 70, 2 to 60, 2 to 50, 2 to 40 or 2 to 30 siRNAs and/or sRNAs of groups a, b or c. In particular, it is preferred when a nucleic acid of group d. contains the nucleotide sequences of 2 to 20 or 2 to 10 siRNAs and/or sRNAs of groups a, b, or c. In another embodiment of the invention, it is preferred if the nucleic acid of group d. contains the nucleotide sequences of more than 2 siRNAs and/or sRNAs of groups a, b, or c, i.e., at least 3, 4, 5, 6, 7, 8, 9, 10 or more (up to 100) sRNAs of groups a, b, or c.

According to group e, the nucleic acid is a single-stranded DNA containing a sequence of 12 or more nucleotides that is homologous (deoxyribonucleotides instead of ribonucleotides) to one of the nucleotide sequences of the single-stranded RNAs of groups a or b.

Sequence homology means the similarity of nucleotide or amino acid sequences due to identical chemical building blocks in more or less large sections of the molecular chains of peptides, RNA or DNA. If molecular chains are completely identical, there is 100% sequence homology. In the present case, homology means 100% sequence homology over 12 or more nucleotides contained in the single-stranded DNA to one of the single-stranded RNAs of groups a. or b., the DNA consisting of deoxyribonucleotides and the RNA consisting of ribonucleotides.

Group e. comprises the antisense deoxyribonucleic acid (DNA) oligonucleotides (ASO) provided by means of the invention, the sequence of which can be derived from the esiRNAs/ERNAs identified in the scope of the invention. Using analogous terminology to the term esiRNAs/ERNAs, the ASOs derived here from the sequences of the single-stranded RNA components of the esiRNAs/ERNAs of groups a. and b. are accordingly referred to as eASOs. eASOs contain DNA sequences (i.e., deoxynucleotides instead of ribonucleotides; thymidine instead of uridine) which are homologous to the single-stranded RNA components of the esiRNAs/ERNAs of groups a. and b. and can therefore also hybridize via corresponding base pairing to the respective target sites on the a-sites of the target RNAs and become active. The operating principle of ASO is similar but not identical to that of sRNAs. As detailed above, ASOs bind via base pairing (hybridization) to completely or partially complementary regions (target sites) of a target RNA. However, the inactivating mode of action of ASOs on a target RNA does not take place via RNAi, as in sRNAs, but rather via antisense processes, e.g., inhibition of translation or endonucleolytic degradation by RNases of the RNase H type (see above).

According to group f., the nucleic acid of the invention is a single-stranded DNA which has modifications at 1 to 7 positions in the nucleotide sequence. The nucleic acids of group f, thus represent variants of the nucleic acids of group e. Within the meaning of the invention, “modification” means “base substitution,” i.e., that a base selected from adenine (A), thymine (T), guanine (G) and cytosine (C) can be substituted by any of the other bases. Such base substitutions can occur at 1 to 7 positions in the nucleotide sequence of a nucleic acid of group e. The base substitutions are preferably independent of each other at each position, i.e., each of the 1 to 7 positions of the nucleotide sequence of a nucleic acid of group e. can be substituted independently of each other by any of the other bases.

According to one aspect of the invention, the nucleic acid is a nucleic acid for the protection of plants against the plant pathogen cucumber mosaic virus (CMV).

Preferably, the nucleic acid for protection against CMV is a nucleic acid which is directed against a target RNA of CMV. It is particularly preferred when the nucleic acid for protection against CMV is a nucleic acid which is directed against a target RNA of CMV, wherein the target RNA of CMV is selected from the target RNAs having SEQ ID NOs: 189 and 190.

A nucleic acid which is directed against a target RNA of CMV selected from SEQ ID NOs: 189 and 190 is preferably a ribonucleic or deoxyribonucleic acid which contains or consists of at least one nucleic acid selected from the group consisting of the nucleic acids having SEQ ID NOs: 1 to 92.

The target RNA of CMV having SEQ ID NO: 189 is hereinafter also referred to as “CMV RNA 2.” The target RNA of CMV having SEQ ID NO: 190 is hereinafter also referred to as “CMV RNA 3.”

As described in application example 1, nucleic acids against CMV RNA 2 (SEQ ID NO: 189) were identified, a significant proportion of which induce efficient hydrolysis in the slicer (cleavage) assay (at least 25%, preferably at least 50%, more preferably at least 75%, particularly preferably at least 90%, particularly preferably at least 95% of the original quantity used in the assay in question) of the target RNA (CMV RNA 2) and protect plants against CMV infections (see also FIGS. 2-4). The screening was carried out with both AGO1 (L version; Gursinsky et al., 2015) and AGO2 from Nicotiana benthamiana (Nb).

As described in application example 1, nucleic acids against CMV RNA 3 (SEQ ID NO: 190) were identified, some of which induce efficient hydrolysis in the slicer (cleavage) assay (at least 25% of the original quantity used in the assay in question) of the target RNA (CMV RNA 3) and protect plants against CMV infections (see also FIGS. 5 and 6). The screening was carried out with both AGO1 (L version; Gursinsky et al., 2015) and AGO2 from Nicotiana benthamiana (Nb).

A nucleic acid which is directed against a target RNA, CMV RNA 2, of CMV having SEQ ID NO: 189 is preferably a ribonucleic or deoxyribonucleic acid which contains or consists of at least one nucleic acid selected from the group consisting of the nucleic acids having SEQ ID NOs: 1 to 52. A particularly suitable nucleic acid which is directed against a target RNA, CMV RNA 2, of CMV having SEQ ID NO: 189 is a ribonucleic or deoxyribonucleic acid which contains or consists of at least one nucleic acid selected from the group consisting of the nucleic acids having SEQ ID NOs: 1-4, 6-11, 14-17, 21-25, 27-30, 32-37, 40-43, and 47-51.

A nucleic acid which is directed against a target RNA, CMV RNA 2, of CMV having SEQ ID NO: 189 and which was identified in the screening with AGO1 is preferably a ribonucleic or deoxyribonucleic acid which contains or consists of at least one nucleic acid selected from the group consisting of the nucleic acids having SEQ ID NOs: 1 to 12 and SEQ ID NOs: 27 to 38, preferably having SEQ ID NOs: 1 to 4, 6 to 11, 27 to 30 and 32 to 37.

A nucleic acid which is directed against a target RNA, CMV RNA 2, of CMV having SEQ ID NO: 189 and which was identified in the screening with AGO2 is preferably a ribonucleic or deoxyribonucleic acid which contains or consists of at least one nucleic acid selected from the group consisting of the nucleic acids having SEQ ID NOs: 13 to 26 and SEQ ID NOs: 39 to 52, preferably having SEQ ID NOs: 14 to 17, 21 to 25, 40 to 43 and 47 to 51.

A nucleic acid which is directed against a target RNA, CMV RNA 3, of CMV having SEQ ID NO: 190 is preferably a ribonucleic or deoxyribonucleic acid which contains or consists of at least one nucleic acid selected from the group consisting of the nucleic acids having SEQ ID NOs: 53 to 92.

A nucleic acid which is directed against a target RNA, CMV RNA 3, of CMV having SEQ ID NO: 190 and which was identified in the screening with AGO1 is preferably a ribonucleic or deoxyribonucleic acid which contains or consists of at least one nucleic acid selected from the group consisting of the nucleic acids having SEQ ID NOs: 53-55, 59, 63, 64, 66, 67, 70-75, 79, 83, 84, 86, 87, 90-92 preferably having SEQ ID NOs: 53, 55, 59, 63, 64, 66, 70, 73, 75, 79, 83, 84, 86 and 90.

A nucleic acid which is directed against a target RNA, CMV RNA 3, of CMV having SEQ ID NO: 190 and which was identified in the screening with AGO2 is preferably a ribonucleic or deoxyribonucleic acid which contains or consists of at least one nucleic acid selected from the group consisting of the nucleic acids having SEQ ID NOs: 56 to 58, 60 to 62, 65, 68, 69, 76 to 78, 80 to 82, 85, 88, 89 preferably having SEQ ID NOs: 56-58, 60-62, 65, 69, 76-78, 80-82, 85 and 89.

According to a further aspect of the invention, the nucleic acid is a nucleic acid for the protection of plants against the plant pathogen Meloidogyne incognita.

Preferably, the nucleic acid for protection against Meloidogyne incognita is a nucleic acid which is directed against a target RNA of Meloidogyne incognita. It is particularly preferred when the nucleic acid for protection against Meloidogyne incognita is a nucleic acid which is directed against a target RNA of Meloidogyne incognita, wherein the target RNA of Meloidogyne incognita is selected from the target RNAs having SEQ ID NOs: 191, 192 and 193.

The target RNA of Meloidogyne incognita having SEQ ID NO: 191 is hereinafter also referred to as “SPF.” The target RNA of Meloidogyne incognita having SEQ ID NO: 192 is hereinafter also referred to as “INT.” The target RNA of Meloidogyne incognita having SEQ ID NO: 193 is hereinafter also referred to as “ACT.”

As described in application example 3, nucleic acids against SPF (SEQ ID NO: 191) were identified, a significant proportion of which induce efficient hydrolysis in the slicer (cleavage) assay and in vivo after being taken up into the nematodes (at least 25% of the original quantity used in the assay in question) of the target RNA (SPF) and protect plants against Meloidogyne incognita infections (see also FIGS. 16A and B). The screening was carried out with the AGO2 protein of Nicotiana benthamiana (Nb).

As described in application example 3, nucleic acids against INT (SEQ ID NO: 192) were identified, a significant proportion of which induce efficient hydrolysis in the slicer (cleavage) assay and in vivo after being taken up into the nematodes (at least 25% of the original quantity used in the assay in question) of the target RNA (SPF) and protect plants against Meloidogyne incognita infections (see also FIGS. 17A and B). The screening was carried out with the AGO2 protein of Nicotiana benthamiana (Nb).

As described in application example 3, nucleic acids against ACT (SEQ ID NO: 193) were identified, a significant proportion of which induce efficient hydrolysis in the slicer (cleavage) assay and in vivo after being taken up into the nematodes (at least 25% of the original quantity used in the assay in question) of the target RNA (SPF) and protect plants against Meloidogyne incognita infections (see also FIGS. 17A and B). The screening was carried out with the AGO2 protein of Nicotiana benthamiana (Nb).

A nucleic acid which is directed against a target RNA of Meloidogyne incognita selected from SEQ ID NOs: 191, 192 and 193 is preferably a ribonucleic or deoxyribonucleic acid which contains or consists of at least one nucleic acid selected from the group consisting of the nucleic acids having SEQ ID NOs: 93 to 120.

A nucleic acid which is directed against a target RNA of Meloidogyne incognita, SPF, having SEQ ID NO: 191 is preferably a ribonucleic or deoxyribonucleic acid which contains or consists of at least one nucleic acid selected from the group consisting of the nucleic acids having SEQ ID NOs: 93 to 95 and 107 to 109.

A nucleic acid which is directed against a target RNA of Meloidogyne incognita, INT, having SEQ ID NO: 192 is preferably a ribonucleic or deoxyribonucleic acid which contains or consists of at least one nucleic acid selected from the group consisting of the nucleic acids having SEQ ID NOs: 96 to 99 and 110 to 113.

A nucleic acid which is directed against a target RNA of Meloidogyne incognita, ACT, having SEQ ID NO: 193 is preferably a ribonucleic or deoxyribonucleic acid which contains or consists of at least one nucleic acid selected from the group consisting of the nucleic acids having SEQ ID NOs: 100 to 106 and 114 to 120.

According to a further aspect of the invention, the nucleic acid is a nucleic acid for the protection of plants against the plant pathogen Botrytis cinerea.

Preferably, the nucleic acid for protection against Botrytis cinerea is a nucleic acid which is directed against a target RNA of Botrytis cinerea. It is particularly preferred when the nucleic acid for protection against Botrytis cinerea is a nucleic acid which is directed against a target RNA of Botrytis cinerea, wherein the target RNA of Botrytis cinerea is selected from the target RNAs having SEQ ID NOs: 194, 195, 196, 197, 198 and 199.

The target RNA of Botrytis cinerea having SEQ ID NO: 194 is hereinafter also referred to as “VDS.” The target RNA of Botrytis cinerea having SEQ ID NO: 195 is hereinafter also referred to as “DCTN.” The target RNA of Botrytis cinerea having SEQ ID NO: 196 is hereinafter also referred to as “SAC.” The target RNA of Botrytis cinerea having SEQ ID NO: 197 is hereinafter also referred to as “ERG.” The target RNA of Botrytis cinerea having SEQ ID NO: 198 is hereinafter also referred to as “EF.” The target RNA of Botrytis cinerea having SEQ ID NO: 199 is hereinafter also referred to as “CHS.”

As described in application example 4, nucleic acids against VDS (SEQ ID NO: 194) were identified, a significant proportion of which induce efficient hydrolysis in the slicer (cleavage) assay (at least 25% of the original quantity used in the assay in question) of the target RNA (VDS) and protect plants against Botrytis cinerea infections (see also FIGS. 18 and 19 A and B). The screening was carried out with the AGO1 protein of Colletotrichum graminicula (Cg).

As described in application example 4, nucleic acids against DCTN (SEQ ID NO: 195) were identified, a significant proportion of which induce efficient hydrolysis in the slicer (cleavage) assay (at least 25% of the original quantity used in the assay in question) of the target RNA (DCTN) and protect plants against Botrytis cinerea infections (see also FIGS. 18 and 19 A and B). The screening was carried out with the AGO1 protein of Colletotrichum graminicula (Cg).

As described in application example 4, nucleic acids against SAC (SEQ ID NO: 196) were identified, a significant proportion of which induce efficient hydrolysis in the slicer (cleavage) assay (at least 25% of the original quantity used in the assay in question) of the target RNA (SAC) and protect plants against Botrytis cinerea infections (see also FIGS. 18 and 19 A and B). The screening was carried out with the AGO1 protein of Colletotrichum graminicula (Cg).

As described in application example 4, nucleic acids against ERG (SEQ ID NO: 197) were identified, a significant proportion of which induce efficient hydrolysis in the slicer (cleavage) assay (at least 25% of the original quantity used in the assay in question) of the target RNA (ERG) and protect plants against Botrytis cinerea infections (see also FIGS. 18 and 19 A and B). The screening was carried out with the AGO1 protein of Colletotrichum graminicula (Cg).

As described in application example 4, nucleic acids against EF (SEQ ID NO: 198) were identified, a significant proportion of which induce efficient hydrolysis in the slicer (cleavage) assay (at least 25% of the original quantity used in the assay in question) of the target RNA (ERG) and protect plants against Botrytis cinerea infections (see also FIGS. 18 and 19 A and B). The screening was carried out with the AGO1 protein of Colletotrichum graminicula (Cg).

As described in application example 4, nucleic acids against CHS (SEQ ID NO: 199) were identified, a significant proportion of which induce efficient hydrolysis in the slicer (cleavage) assay (at least 25% of the original quantity used in the assay in question) of the target RNA (CHS) and protect plants against Botrytis cinerea infections (see also FIGS. 18 and 19 A and B). The screening was carried out with the AGO1 protein of Colletotrichum graminicula (Cg).

A nucleic acid which is directed against a target RNA of Botrytis cinerea selected from SEQ ID NOs: 194, 195, 196, 197, 198 and 199 is preferably a ribonucleic or deoxyribonucleic acid which contains or consists of at least one nucleic acid selected from the group consisting of the nucleic acids having SEQ ID NOs: 121 to 180, preferably having SEQ ID NOs: 124, 126-132, 134-138, 140-150, 154, 156-162, 164-168 and 170-180.

A nucleic acid which is directed against a target RNA of Botrytis cinerea, VDS, having SEQ ID NO: 194 is preferably a ribonucleic or deoxyribonucleic acid which contains or consists of at least one nucleic acid selected from the group consisting of the nucleic acids having SEQ ID NOs: 121, 122, 151 and 152.

A nucleic acid which is directed against a target RNA of Botrytis cinerea, DCTN, having SEQ ID NO: 195 is preferably a ribonucleic or deoxyribonucleic acid which contains or consists of at least one nucleic acid selected from the group consisting of the nucleic acids having SEQ ID NOs: 123-131 and 153-161, preferably from SEQ ID NOs: 124, 126-131, 154 and 156-161.

A nucleic acid which is directed against a target RNA of Botrytis cinerea, SAC, having SEQ ID NO: 196, is preferably a ribonucleic or deoxyribonucleic acid which contains or consists of at least one nucleic acid selected from the group consisting of the nucleic acids having SEQ ID NOs: 132-140 and 162-170, preferably from SEQ ID NOs: 132, 134-138, 140, 162, 164 to 168 and 170.

A nucleic acid which is directed against a target RNA of Botrytis cinerea, ERG, having SEQ ID NO: 197 is preferably a ribonucleic or deoxyribonucleic acid which contains or consists of at least one nucleic acid preferably selected from the group consisting of the nucleic acids having SEQ ID NOs: 141 to 149 and 171 to 179.

A nucleic acid which is directed against a target RNA of Botrytis cinerea, EF, having SEQ ID NO: 198 is preferably a ribonucleic or deoxyribonucleic acid which contains or consists of at least one nucleic acid preferably selected from the group consisting of the nucleic acids having SEQ ID NOs: 150 and 180.

In a further aspect of the invention, the nucleic acid is a double-stranded RNA, this double-stranded RNA preferably containing nucleotide sequences consisting of what are referred to as “pseudo-siRNA sequences” and sequences of at least two and at most an infinite number of sRNAs according to group c. Preferably, the double-stranded RNA contains nucleotide sequences consisting of pseudo-siRNA sequences and sequences of 2 to 10,000 sRNAs according to group c. Particularly preferably, the double-stranded RNA contains nucleotide sequences consisting of pseudo-siRNA sequences and sequences of 2 to 5,000 or 2 to 2,500 sRNAs according to group c. Very particularly preferably, the double-stranded RNA contains nucleotide sequences consisting of pseudo-siRNA sequences and sequences of 2 to 1,000, 2 to 500, 2 to 250, 2 to 100 or 2 to 50 sRNAs according to group c. These are nucleic acids of group d., “edsRNAs,” as described above and in application example 2.

What is referred to as a “pseudo-siRNA sequence” or “pseudo-siRNA” within the meaning of the invention is a double-stranded ribonucleotide sequence of any composition which, according to the planned processing of the respective edsRNA by Dicer enzymes or DCLs, is 21, 22, 23 or 24 nt long (see also application example 2). The name “pseudo-siRNA sequence” or pseudo-siRNA was chosen to express the fact that this is a double-stranded ribonucleotide sequence that is constructed similarly to siRNA but does not actually function as an siRNA, but rather has other functions described below. Pseudo-siRNA sequences are placed at the termini of a double-stranded RNA (edsRNA) engineered according to the invention. Their presence forces the Dicers or DCLs active on this RNA into “synchronized processing.” As described in detail in exemplary embodiment 2, synchronized processing means that, depending on the position and length of the pseudo-siRNA sequences and accordingly on the activity of the Dicer/DCLs involved, the endonucleolytic cleavage of the double-stranded RNA occurs in such a way that the sRNAs preferably generated in the process have the same length as the pseudo-siRNAs. The fact that this occurs in this way was demonstrated according to the invention (FIG. 14). Furthermore, functional elements within the meaning of the invention can be incorporated in pseudo-siRNA sequences: these can be elements which are important for the transcription and processing of the RNA in question. These can be, for example, regions of a transcription promoter or terminator, but they can also be transport signals or parts of a ribozyme which generates the correct 5′ or 3′ end of the RNAs in question by self-splicing (self-catalyzed cleavage).

Transcription promoters are signal sequences in a DNA double strand, one strand of which (template strand) encodes an RNA molecule. The transcription promoter is recognized by a DNA-dependent RNA polymerase complex and, due to association with this promoter sequence, the RNA polymerase complex can initiate the transcription (synthesis) of an RNA molecule, the nucleotide sequence of which is complementary to the DNA template strand. Transcription activators that bind to the RNA polymerase complex and/or other DNA sequences can specifically induce transcription. Examples of transcription promoters are viral promoters such as the promoter of the T7 phage (T7 promoter). Examples of cellular promoters include the Pol I and Pol II promoters. Other examples of common viral promoters are: T3 promoter (promoter of the T3 phage), SP6 promoter (promoter of the SP6 phage), CMV promoter (promoter of human cytomegalovirus). Other examples of common cellular promoters are: GAL promoters, LAC4 promoters, actin promoter, Pol III promoter. Other suitable transcription promoters are known to a person skilled in the art.

Transcription terminators are nucleotide sequences that, like transcription promoters, are encoded by RNA-encoding DNA. When these sequences are transcribed by the RNA polymerase complex, protein-RNA complexes are formed, which cause the RNA polymerase complex to terminate transcription. One example of a transcription terminator is the transcription terminator of vesicular stomatitis virus (VSV). Other suitable transcription terminators are known to a person skilled in the art.

Transport signals in RNA molecules require the binding of proteins that enable the targeted transport of these RNA molecules into or out of specific cell compartments. One example is nuclear RNA export signals, which enable the export of RNA molecules from the nucleus into the cell cytoplasm. Other transport signals are known to a person skilled in the art.

Ribozymes are catalytically active RNA molecules that catalyze chemical reactions like enzymes. Examples include Hammerhead (hammerhead; HH) ribozymes (Meyer and Masquida 2014) or the Hepatitis Delta Virus (HDV) ribozyme (Avis et al. 2012). These ribozymes can independently catalyze endonucleolytic cleavage of the RNA molecule of which they are a constituent (self-cleavage or self-splicing).

The basic principle of the structure of edsRNAs designed within the meaning of the invention is thus presented as follows (see also FIGS. 8-11):

The sequence contains at least one pseudo-siRNA sequence which, as stated, enables synchronized processing by Dicer/DCLs. The sequence of the edsRNAs also contains a number of sequences, “in a row” from 5′ to 3′, of esiRNAs/ERNAs or of other sRNAs, for example miRNAs, derived from esiRNAs/ERNAs. These sequences can originate from various “eNA screens,” and the esiRNA/ERNAs or sRNAs derived therefrom can accordingly be active in different AGO/RISC. The esiRNA/ERNAs or sRNAs derived therefrom that constitute an edsRNA can thus be directed against different target RNAs that originate from one, or from different, organism(s).

An edsRNA can be generated in two ways: from two independently transcribed complementary RNA molecules or from one transcribed RNA molecule containing two complementary sections (FIG. 8). In the latter case, the two complementary sections of the transcribed RNA are connected to each other by a spacer and form a hairpin. The spacer is a sequence of any composition having a minimum length of 4 nucleotides (see definition above) which, however, can contain functional regions such as ribozymes (such as HH or HDV ribozymes), transcription promoters or transcription terminators, transport signals or splice sites for the specific purpose of edsRNA construction.

Splice sites are sequence motifs in regions of precursor RNA molecules such as introns that are recognized by the splicing machinery of a cell. The splicing machinery catalyzes the complete or partial removal of the intron sequence. In addition to the function of connecting the two complementary single-stranded components of a dsRNA, the spacer sequence also serves further purposes due to the presence of these elements: if it contains, for example, splice sites, the spacer can be truncated by the cell's splicing machinery in the course of in vivo RNA expression. Since, in contrast to the double-stranded RNA regions, the spacer is sensitive to ribonucleases (such as the single-strand-specific RNases T1 or A) that are present in the cell, this sequence can also be completely removed by these RNases (see also FIG. 8). Examples of spacers are sequences of introns from mRNA precursor molecules (“pre-mRNAs”), which contain all of the recognition sequences necessary for splicing. These recognition sequences are known to a person skilled in the art.

The transcription of RNAs can take place in vitro or in vivo. The double strand is obtained by hybridization of the complementary RNA strands (FIG. 8). Transcription can take place via a wide variety of promoters (see for example FIG. 11). Transcription termination can take place via any type of transcription terminators (such as VSV transcription terminator, see for example FIG. 11).

Depending on the manner of generation, the termini of the edsRNAs are either blunt or they contain an overhang (−Ü) (FIG. 15). They can be generated in various ways, for example by “run-off transcription” (“causing the RNA polymerase complex to fall off the DNA template”) or by terminating the RNA polymerase complex in question via a transcription terminator or using the activity of ribozymes via self-splicing.

The sequences of both strands are designed in such a way that each of the authentic sRNA guide and passenger strand sequences are generated during processing by DCLs. Processing, which predominantly leads to the generation of the constituent esiRNAs/ERNAs or sRNAs derived therefrom, is ensured by the presence of the pseudo-siRNA sequences and the associated “synchronized processing” by the DCLs/Dicer (see application example 2 and FIG. 8-14).

Thus, in one embodiment of the invention, the double-stranded RNA of the invention can have blunt ends, and in another embodiment it can have overhanging ends.

In a further embodiment of the invention, the double-stranded RNA may contain a spacer. In a further embodiment of the invention, the pseudo-siRNAs and/or spacer in the nucleic acid according to the invention contain elements selected from transcription promoters, transcription terminators, transport signals, splice sites and ribozymes.

In a particularly preferred embodiment of the invention, the double-stranded RNA of the invention is selected from the group consisting of the nucleic acids having SEQ ID NOs: 181, 182, 185, 186 and 200 to 204.

The functionality of such novel edsRNAs and their considerably improved protectiveness against pathogens compared to conventional dsRNAs was demonstrated according to the invention (see e.g., FIGS. 14 and 15).

In a further embodiment, the nucleic acid according to the invention has one or more chemical modifications, wherein the chemical modifications are selected from conjugates such as GalNac, base modifications such as 5-methylcytosine, 2′ sugar modifications such as 2′-O-methyl, 2′-fluoro, 2′-O-methoxyethyl(2′-MOE), cETBNA (ethyl bicylically (S)-bonded), other sugar modifications such as “locked” (LNA) or “unlocked” (UNA), “backbone” modifications such as phosphorothioate (PS) or “peptide nucleic acids” (PNA), and also sugar-phosphate modifications such as morpholino/PMO (phosphorodiamidate morpholino). Other modifications are known to a person skilled in the art.

In a further aspect, the invention relates to a composition comprising at least one, optionally a plurality of, nucleic acid(s) of the invention, as described herein.

The nucleic acids according to the present invention can be used in transgenic form, e.g., HIGS methods for pathogen control, or for the targeted transcriptional and post-transcriptional regulation of gene expression. They are particularly suitable for use in pathogen control in plants/crops. In one embodiment, the invention therefore relates to the use of a nucleic acid or composition according to the invention in plants for the prophylaxis and/or treatment of infestations and/or infections by pathogens, in particular for the prophylaxis and/or treatment of infestations and/or infections by pathogens selected from cucumber mosaic virus, Meloidogyne incognita and Botrytis cinerea.

Transgenic means that the genetic information encoding at least one, optionally a plurality, of the nucleic acid(s) of the invention is stably introduced into the genome of a host organism, preferably a plant, a microorganism or one of the pathogens mentioned, and is produced (expressed) in this organism in an inducible or non-inducible manner via corresponding promoters. If the organism is a plant, said plant can thus be made resistant to infestations and/or infections by one or more of the corresponding pathogens. If this organism is a microorganism such as bacteria or yeast, the corresponding nucleic acid can be produced in these microorganisms.

The nucleic acids according to the present invention can also be used in transient (non-transforming) form, e.g., SIGS methods for pathogen control, or for the targeted transcriptional and post-transcriptional regulation of gene expression. They are particularly suitable for use in pathogen control in plants/crops. In one embodiment, the invention therefore relates to the use of a nucleic acid or composition according to the invention in plants for the prophylaxis and/or treatment of infestations and/or infections by pathogens, in particular for the prophylaxis and/or treatment of infestations and/or infections by pathogens selected from CMV, Meloidogyne incognita and Botrytis cinerea.

In topical/transient applications, one or more nucleic acids of the invention are applied to target organs of a plant such as leaves, stems or roots. The nucleic acids are then either taken up by the plant, e.g., to inhibit the replication of infecting viruses, or attacking (infecting) pathogens such as nematodes and fungi take up the nucleic acids via the surface of the plant to be protected, and these are then active against essential target RNAs of the pathogen in the RNA silencing/RNAi or RNA silencinglantisense mechanism of the organism in question.

In one embodiment, the invention accordingly relates to a composition, comprising at least one nucleic acid of the invention and optionally one or more carrier substances and/or adjuvants, which composition is suitable for administration in/on plants.

The composition is preferably a solution that can be administered in direct form, e.g., as a nutrient solution or as an aerosol/spray. This makes it particularly easy to prevent or treat diseases in plants/crops.

Preferably, the composition comprises at least one physiologically compatible carrier, diluent, and/or adjuvant. The nucleic acids according to the present invention may be contained in a pharmaceutically compatible carrier, e.g., in a conventional medium such as an aqueous saline medium or a buffer solution as a composition for an aerosol/spray. Such a medium may also contain conventional adjuvants such as salts for adjusting osmotic pressure, buffers, preservatives, nanoparticles and the like.

Other suitable compatible carrier substances are known to a person killed in the art, for example, from Remington's Practice of Pharmacy, 13th edition, and J. of. Pharmaceutical Science & Technology, vol. 52, no. 5, September-October, pages. 238-311.

In a particularly preferred embodiment of the invention, the nucleic acids of the invention are used in transgenic or transient form in RNA silencing/RNAi methods, wherein the nucleic acids of the invention are in the form of double-stranded RNA molecules according to group d., characterized in that these are constructed so as to be a double-stranded RNA and this double-stranded RNA contains nucleotide sequences consisting of pseudo-siRNA sequences and sequences of at least two siRNAs or sRNAs according to group a., b. or c., and optionally having blunt or overhanging ends and/or including a spacer and/or the pseudo-siRNA sequences and spacer in the nucleic acid contain elements selected from transcription promoters, transcription terminators, transport signals, splice sites and ribozymes and/or the nucleic acid is selected from the group consisting of the nucleic acids having SEQ ID NOs: 181, 182, 185, 186 and 200 to 204 and are processed to give the relevant sRNAs by Dicer or Dicer-like enzymes. Such a use is described in application example 2.

The listed problems have been solved by the invention described below and the application examples described.

Nucleic acid active agents, esiRNAs/ERNAs and sRNAs and eASOs (eNAs) derived therefrom which can be used in various applications (RNA silencing/RNAi or RNA silencing/antisense methods) as active agents in plant protection against the pathogens Cucumber Mosaic Virus or Meloidogyne incognita or Botrytis cinerea, were newly identified.

The invention further relates to the construction of double-stranded ribonucleic acids (edsRNAs (effective double-stranded RNAs) which are constructed from identified esiRNAs/ERNAs and/or

sRNAs derived therefrom and which can be used in RNAsilencing/RNAi methods as active agents in plant protection against said pathogens.

The invention is explained in more detail below with reference to 19 figures, 8 tables and 4 application examples.

### BRIEF DESCRIPTION OF THE FIGURES

In the figures:

FIG. 1: effective esiRNAs/ERNAs and edsRNAs. Left: schematic depiction of an RNA silencing/RNAi process with a natural siRNA pool generated for example from a viral dsRNA by DCL activity: the siRNA pool contains only a few esiRNAs/ERNAs (labeled with an “e”); the RNA silencing/RNAi is accordingly inefficient. Middle: RNA silencing/RNAi carried out with esiRNAs/ERNAs identified using the “eNA screen”; the RNA silencing/RNAi is efficient. Right: sequence information of the identified esiRNAs/ERNAs is used to generate edsRNA; the RNA silencing/RNAi with these edsRNAs/esiRNAs/ERNAs generated therefrom is also efficient. DCL-Dicer-like proteins; AGO-Argonaute proteins; RISC-RNA induced silencing complex. The siRNA guide strand is highlighted in black for usual siRNAs and red for esiRNAs/ERNAs.

FIG. 2: Slicer assays with esiRNAs/ERNAs identified against CMV RNA 2 (SEQ ID NO: 189). As described in the application examples, “eNA screens” were carried out with CMV RNA 2 (Fny type, here with a double-stranded version of the RNA). In the last step shown here, slicer assays were carried out with the siRNA candidates obtained. It is important to note that here, as in all the following slicer assays shown, all siRNAs tested were directed against the (+)-oriented target RNA. AGO1 or AGO2 mRNA was translated in BYL (Gago-Zachert et al., 2019) in the presence of the siRNA to be tested. The RISCs formed according to the method described were thus programmed with the siRNA to be characterized and the endonucleolytic hydrolysis of a defined quantity of radiolabeled CMV RNA 2 (target RNA) was detected by gel electrophoresis and autoradiography. Asterisks (*) mark the cleavage products of the target RNA generated by the slicer activity of the AGO/RISC. The cleavage efficiency can be quantified using autoradiographic measurement of the quantity of target RNA originally used compared to the negative control and compared to the cleavage products formed (ImageQuantTL; see also table 1). As described in the text, here, as in all the figures shown below, a newly established standardized and stringent form of the slicer assay was used: the formation of the AGO/RISC with the respective siRNA to be tested (10 nm) was carried out in the presence of a 10-fold excess (100 nM) of a nonspecific competitor siRNA (“siR gf698” directed against the mRNA of green fluorescent protein, GFP) (see also application examples). This standardized and stringent type of the slicer assay served for the final definition of an esiRNA/ERNA: those siRNAs that, in a slicer assay carried out in this way, induce the AGO/RISC-mediated hydrolysis of at least 25% of the quantity of target RNA originally used in the assay in question are designated as effective- and therefore as esiRNAs/ERNAs (see also table 1). (A) SiRNA candidates from “eNA screen” tested with AGO1/RISC. (B) SiRNA candidates from “eNA screen” with AGO2/RISC. All esiRNAs/ERNAs identified are summarized in table 1. (C) Schematic depiction of the binding sites of the most efficient siRNAs on CMV RNA 2. Protein-coding regions are depicted as gray boxes. The numbers of the siRNA candidates correspond to the designations given below in the figures, tables and text (with the abbreviation siR or siRCV2).

FIG. 3: Characterization of the protective efficiency of CMV RNA 2-specific esiRNAs/ERNAs in planta. Four- to five-week-old N. benthamiana plants were mechanically inoculated with the genomic CMV RNAs 1, 2 and 3 generated from infectious cDNAs, and also with individual synthetic siRNA candidates, and were monitored for the emergence of CMV-specific symptoms over a period of 35 days (dpi=days post inoculation). (A) Representative images of individual plants illustrate the differences between asymptomatic and symptomatic plants 35 days after inoculation. The number of plants that remained asymptomatic is indicated. (B) The graph shows the percentage of asymptomatic plants over the entire course of the experiment: the trends with AGO1-specific siRNAs are shown in black, with AGO2-specific siRNAs and the siR gf698 control in gray. The results come from three independent plant experiments. The number of plants used is indicated (n=12-15). As shown, esiRNA/ERNAs protect plants very efficiently against CMV infection. (C) Shows agarose gels of an RT-PCR for the detection of viral infection at the RNA level. Leaf discs of asymptomatic and symptomatic plants were collected at 35 dpi, the RNA was extracted, and cDNA was synthesized using reverse transcriptase. A PCR was subsequently performed to detect a conserved sequence present in RNAs 1, 2 and 3 of cucumber mosaic virus. The CMV-specific sequence was amplified using plasmids containing the cDNA sequence of the respective viral RNA (positive control) and in samples of symptomatic plants. As can be seen, it was not possible to obtain any PCR product derived from CMV RNA from the cDNA of asymptomatic plants. The RT-PCR thus confirms the visual classification into symptomatic and asymptomatic plants. The numbers correspond to the numbering of the respective plants from the infection experiment. (−) RT assays (no addition of reverse transcriptase during the reaction) and the “water control” (addition of water instead of cDNA during the PCR reaction) served as negative controls. (M=GeneRuler 100 bp DNA-ladder, Thermo Scientific). The numbers of the siRNA candidates correspond to the designations given above and below in the figures, tables and text (with the abbreviation siR or siRCV2).

Table 1. esiRNA/ERNA candidates against CMV RNA 2 (SEQ ID NO: 189). 21 nt-long siRNAs from CMV RNA 2 identified in the scope of the “eRNA screens” are listed. The respective single-stranded guide and complementary passenger strands are specified. The siRNAs were named siRCV (CV for CMV) corresponding to the respective target RNA (CMV RNA 2) and the position of the 5′ end of the identified guide strand. The siRNAs were classified using slicer assays as described: the percentage quantity of the respective target RNA cleaved by the esiRNAs/ERNAs in the standardized and stringent slicer assay is given. esiRNAs/ERNAs which were identified in the “eNA-screen” according to the definition (see the text) are highlighted in bold: AGO1-selected in black; AGO2-selected in gray. Other siRNAs that were also identified in the screen but did not meet the definition of esiRNAs/ERNAs are shown in normal font. Some of these siRNAs (e.g., siRCV21844 and 2634) served as negative controls in the infection experiments in N. benthamiana, which is why they are listed here. The efficiency in protection experiments with N. benthamiana is given for siRNAs tested in planta (n.a.=not available): the proportion of asymptomatic plants 35 days after infection is given. Patent protection is accordingly preferably sought, according to the claims, for the siRNAs and variants thereof highlighted in bold (see table 6) used against CMV in the form of esiRNAs/ERNAs or sRNAs or eASOs (eNAs) derived therefrom. The respective SEQ ID NOs are given and listed separately as an “addendum to table 1.”

FIG. 4. Efficacy of 21 nt- and 22 nt-long esiRNAs/ERNAs against CMV RNA 2 in vitro and in planta.

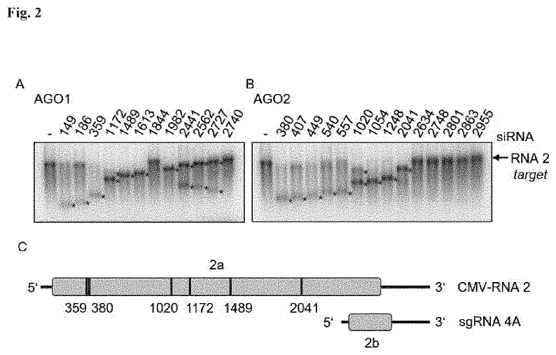

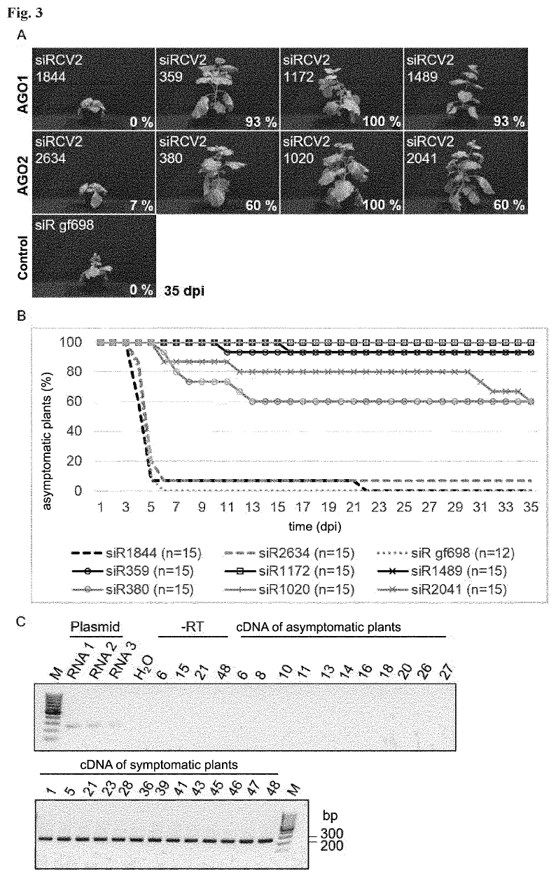

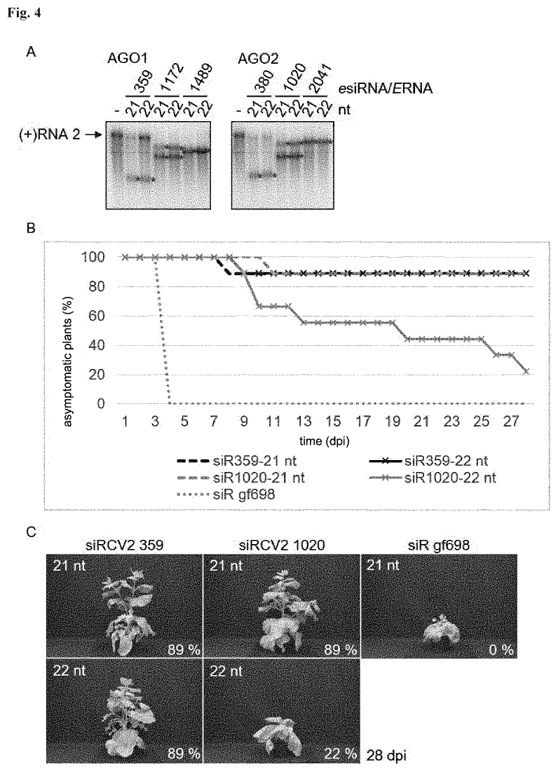

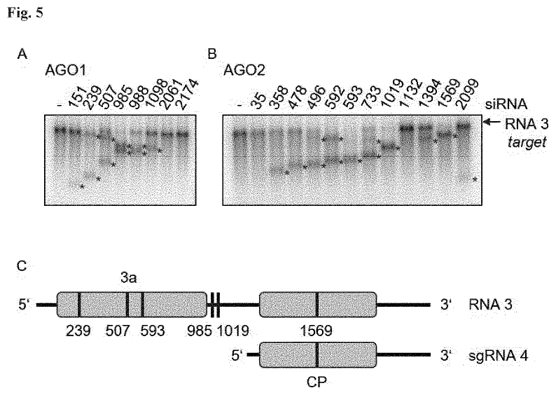

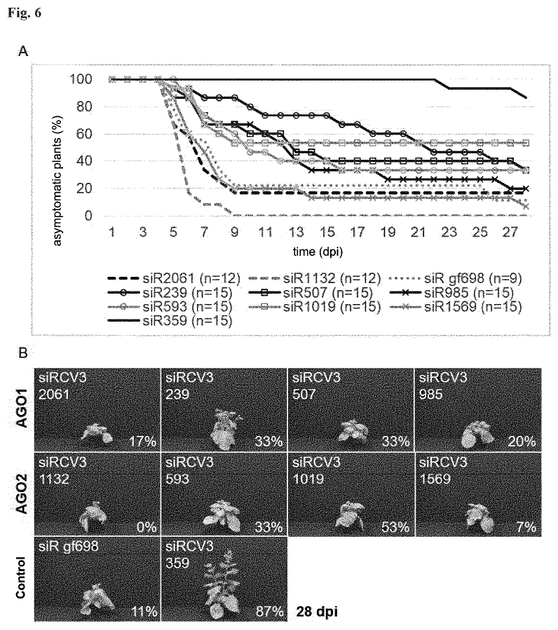

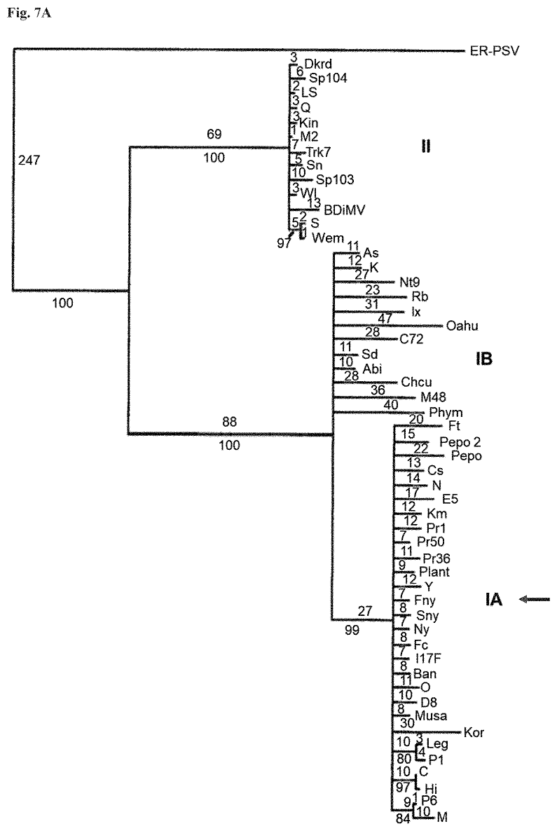

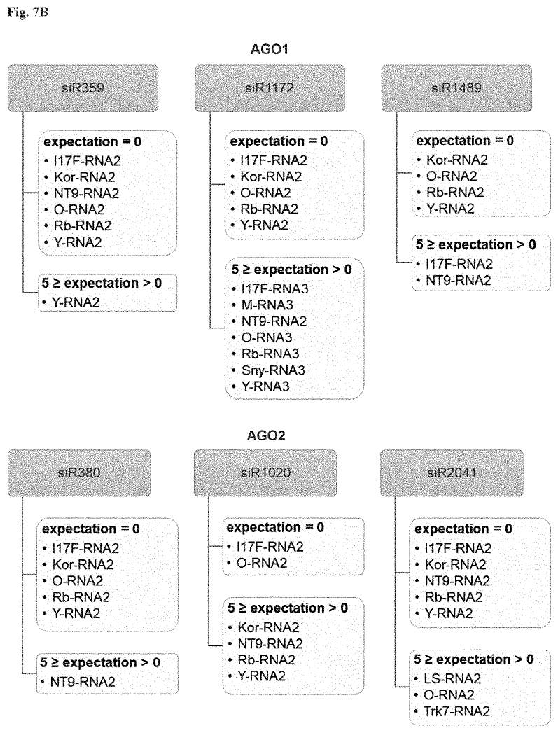

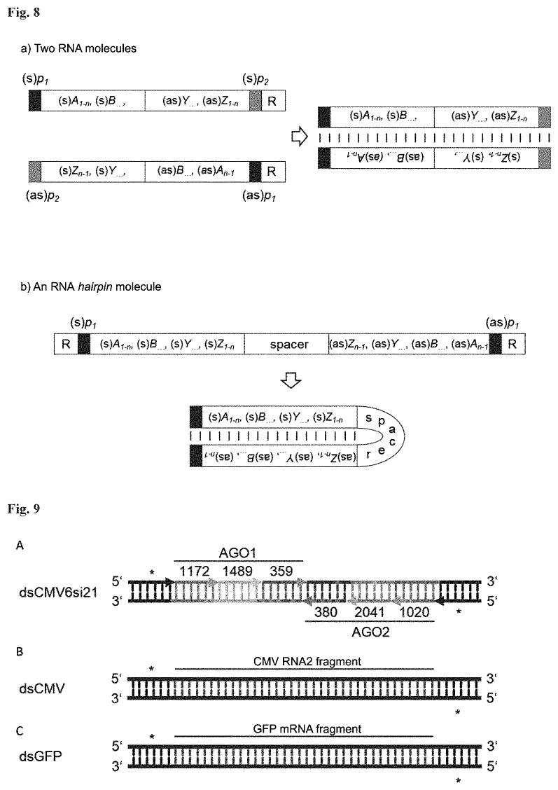

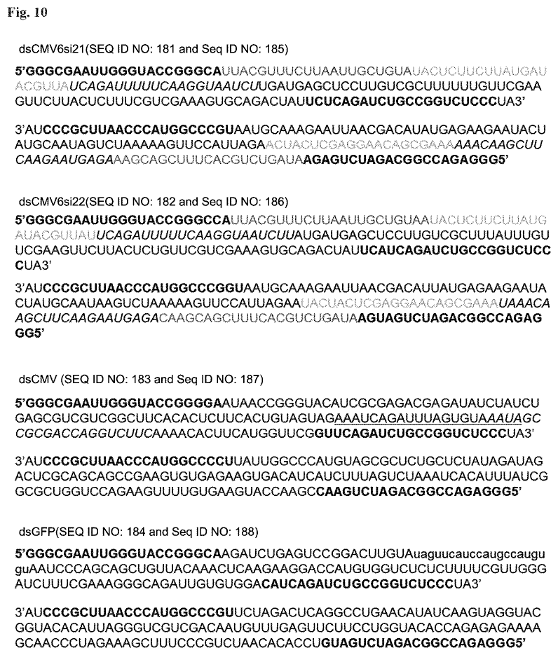

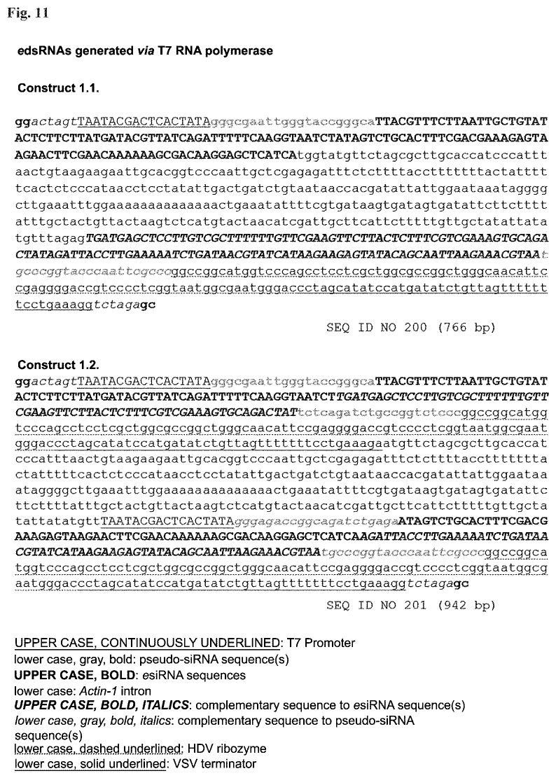

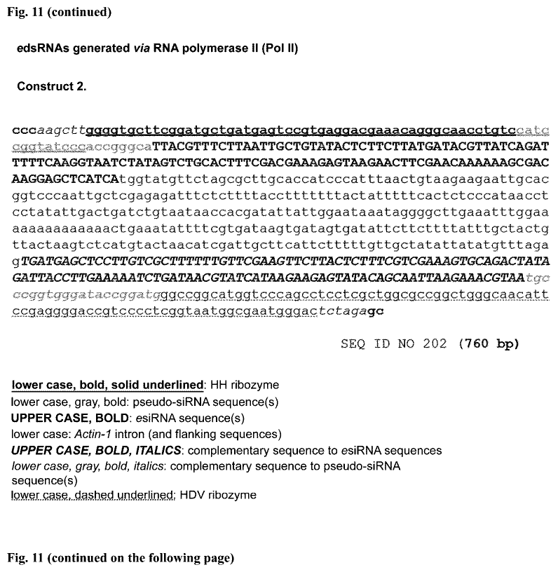

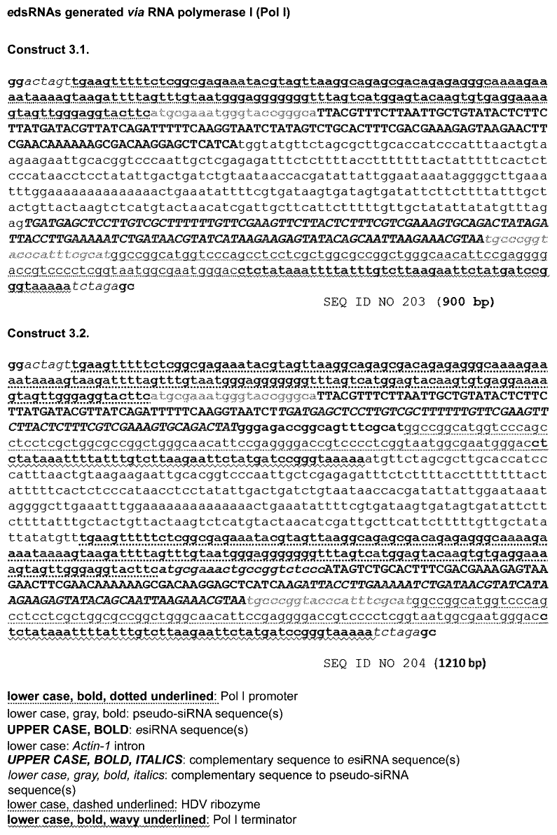

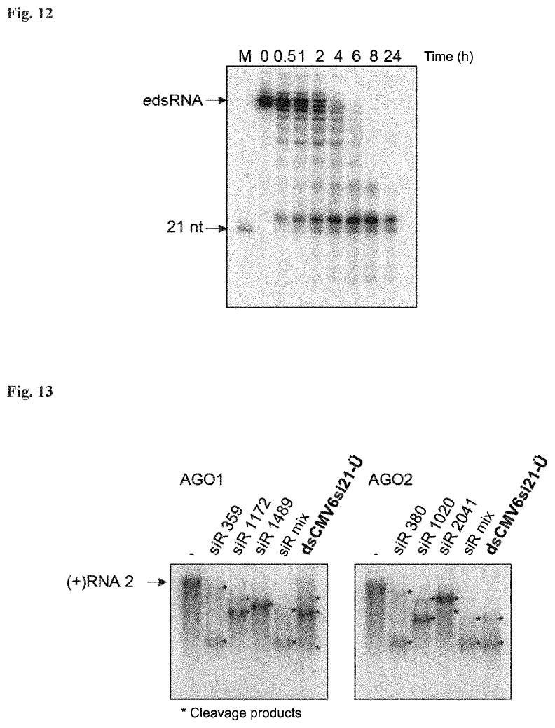

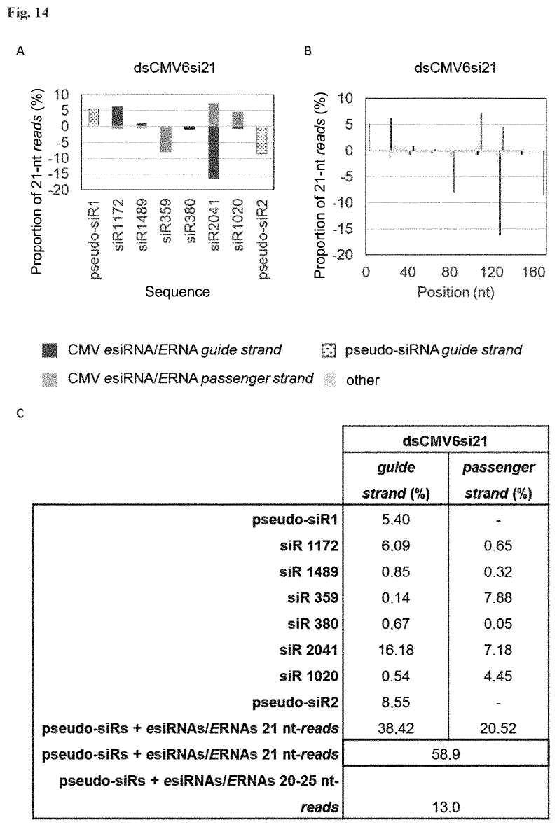

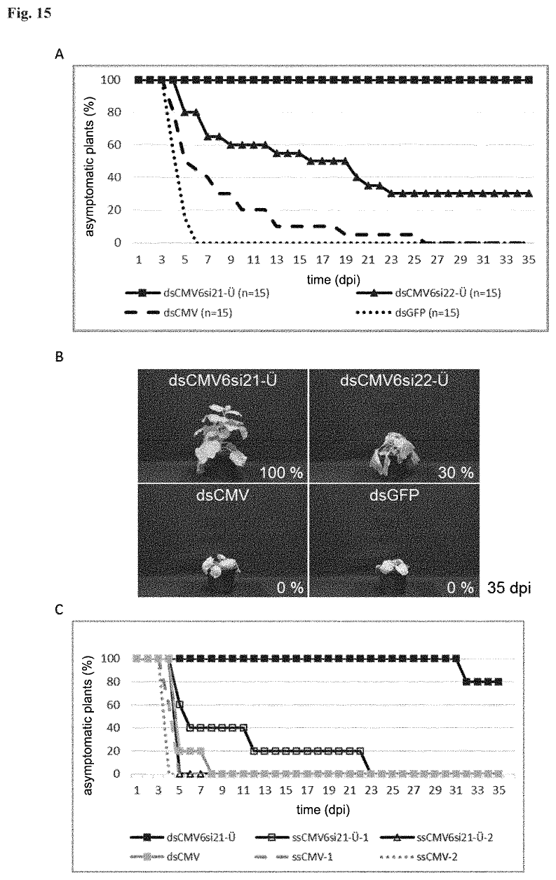

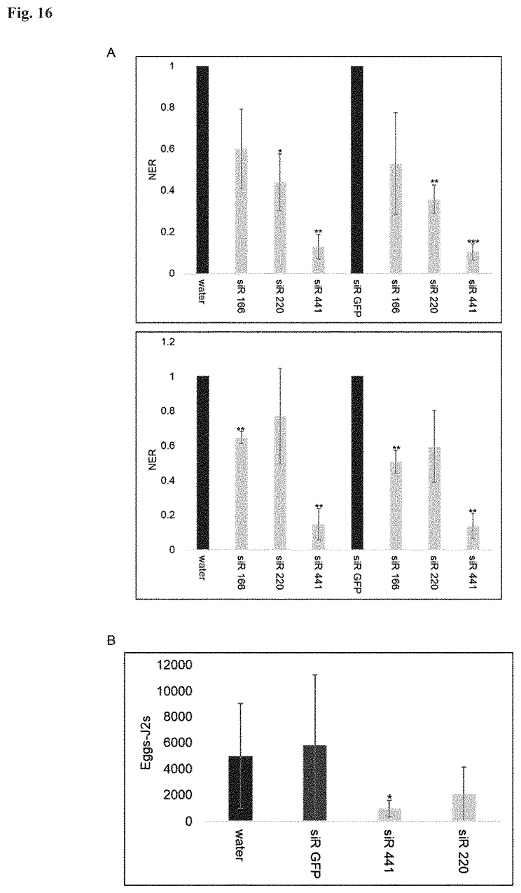

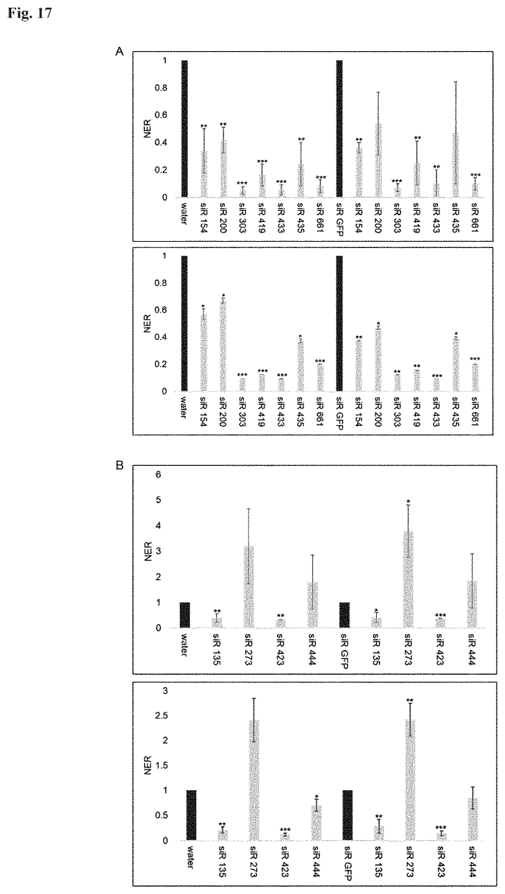

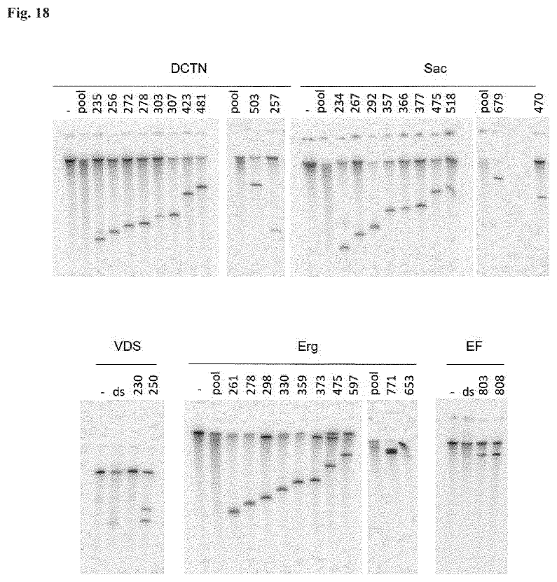

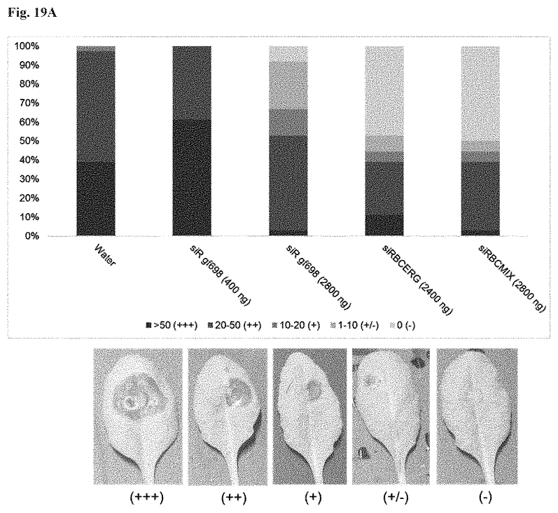

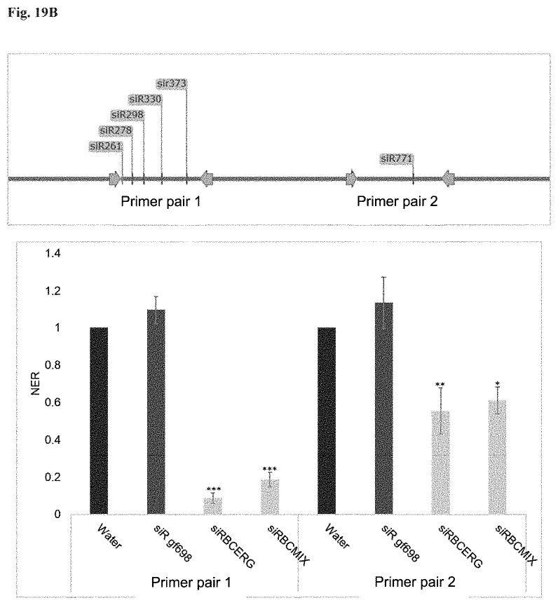

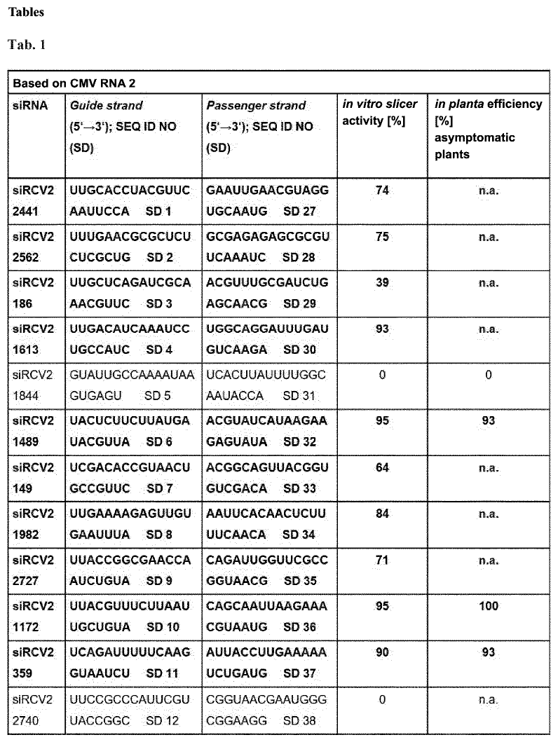

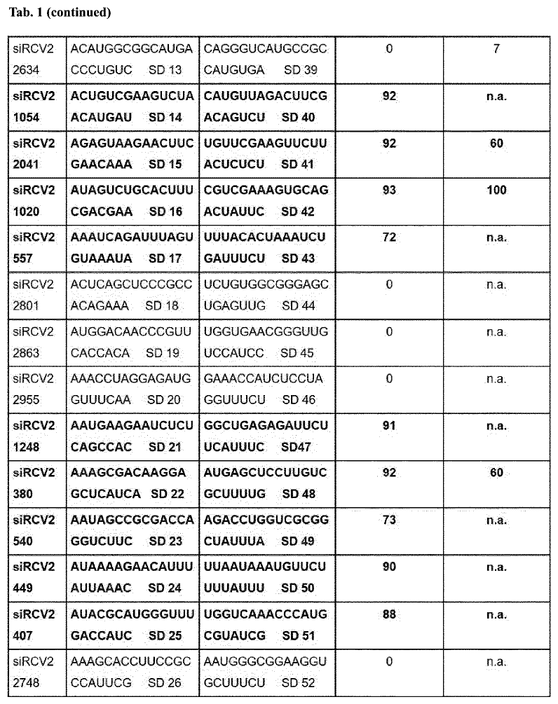

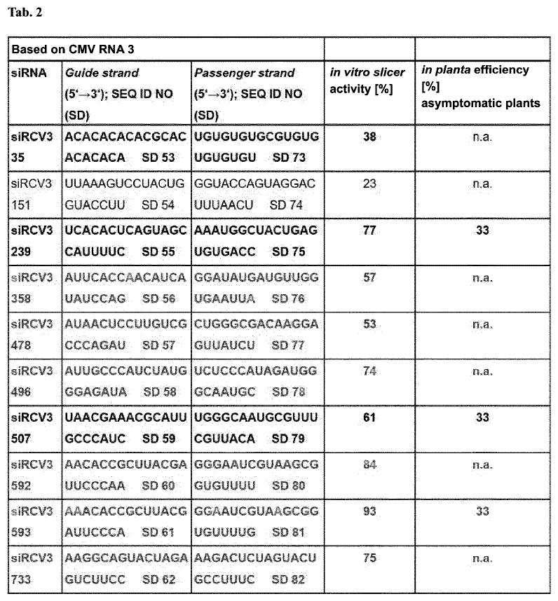

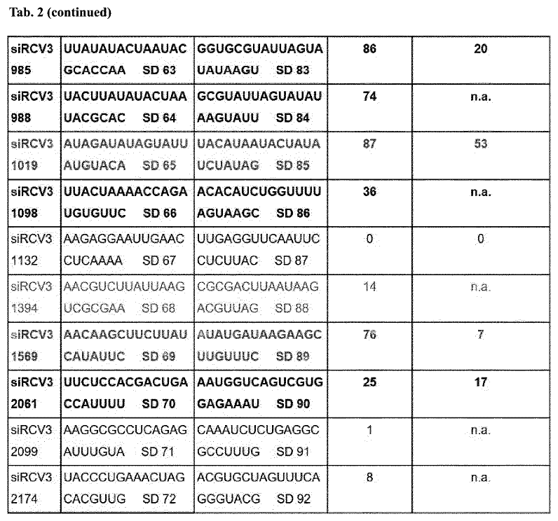

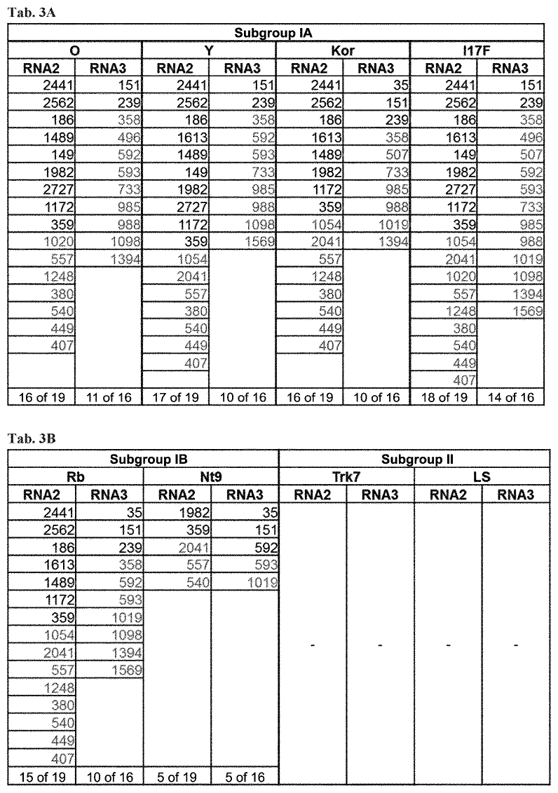

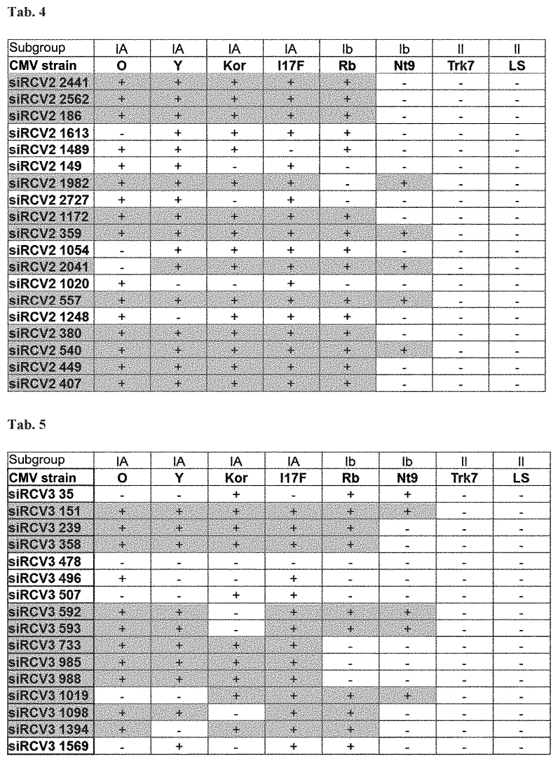

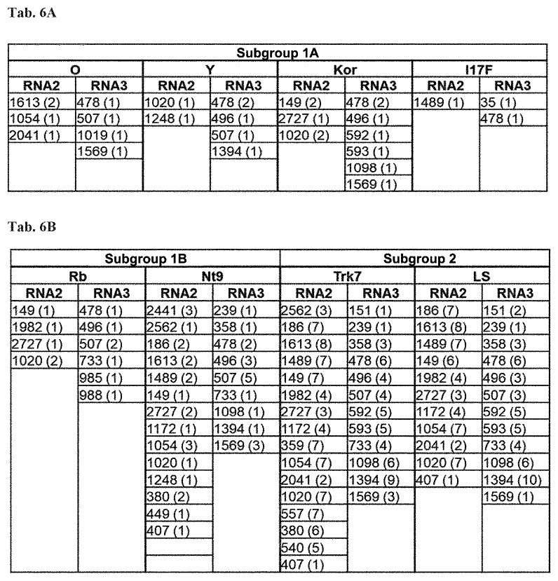

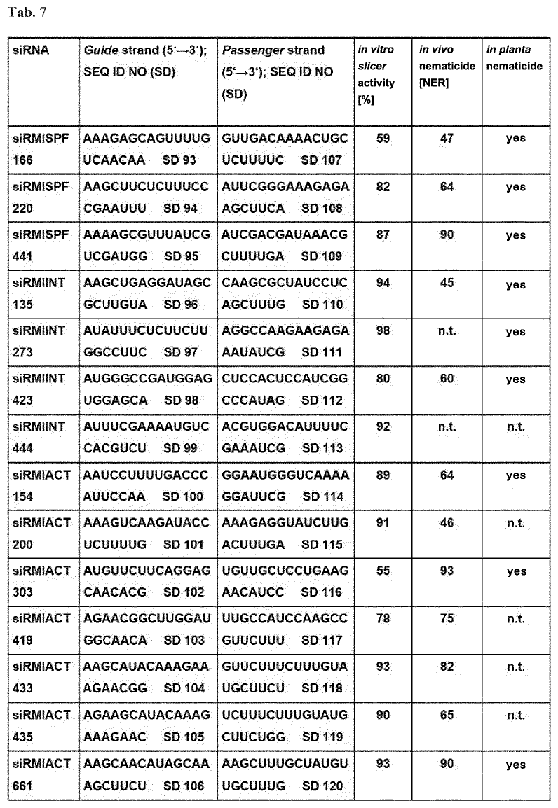

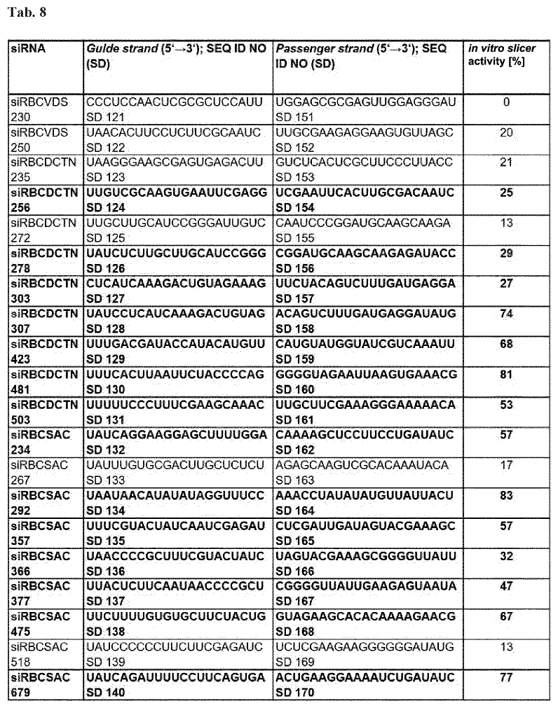

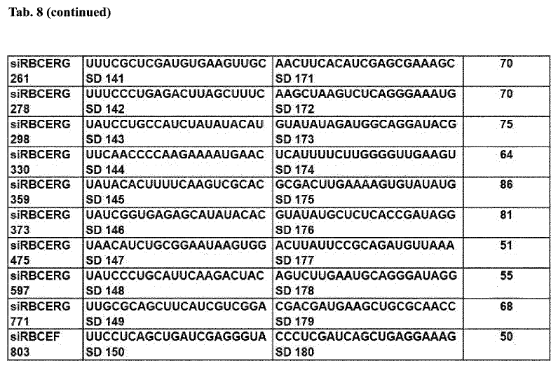

(A) Slicer assay (carried out in a standardized and stringent form as described in FIG. 2) using examples of 21 nt-long esiRNA/ERNAs and 22 nt-long variants derived therefrom. As shown, both variants have high slicer activity. In particular AGO1, but also AGO2, have a slightly higher slicer activity in combination with 21 nt-long esiRNAs/ERNAs than for 22 nt-long esiRNAs/ERNAs. In contrast, the slicer activity of AGO2 in combination with 21 nt- or 22 nt-long esiRNAs/ERNAs differs only minimally. Asterisks (*) mark the cleavage products generated by the slicer activity of the AGO/RISC. (B) Comparison of the protective effect of 21 nt- and 22 nt-long siRNAs (examples) in planta. Four- to five-week-old N. benthamiana plants were mechanically co-inoculated with 21 nt- and 22 nt-long synthetic esiRNAs/ERNAs and the genomic CMV RNAs. The percentage of asymptomatic plants (dpi=days post inoculation) is shown. Both esiRNA/ERNA versions have a significant antiviral effect. (C) Representative plant images 28 days after mechanical co-inoculation. The percentage of asymptomatic plants per siRNA is given. Nine plants each were used for the CMV-specific siRNAs and three plants each for the siR gf698 control. All further controls were carried out similarly to FIG. 3. The numbers of the siRNA candidates correspond to the designations given above and below in the figures, tables and text (with the abbreviation siR or siRCV).

FIG. 5. Slicer assay with esiRNAs/ERNAs identified against CMV RNA 3 (SEQ ID NO: 190). As described in the application examples, “eNA screens” were carried out with a double-stranded version of CMV RNA 3 (Fny type) and in the final step slicer assays were carried out with the siRNA candidates obtained (see also the analogous description in FIG. 2). AGO1 or AGO2 mRNA was translated in BYL in the presence of the siRNA to be tested. The respective RISCs were thus programmed with the siRNA to be characterized and the endonucleolytic hydrolysis of the radiolabeled CMV target RNA 3 was detected by agarose gel electrophoresis and autoradiography. Asterisks (*) mark the cleavage products generated by the slicer activity of the AGO/RISC. The standardized and stringent type of the slicer assay served for the final definition of an esiRNA/ERNA: those siRNAs that, in a slicer assay carried out in this way, induce the AGO/RISC-mediated hydrolysis of at least 25% of the quantity of target RNA originally used in the assay in question are designated as effective—and therefore as esiRNAs/ERNAs (see also table 2). (A) SiRNA candidates from screen with AGO1. (B) SiRNA candidates from screen with AGO2. All esiRNAs/ERNAs identified are summarized in table 2. (C) Schematic depiction of the binding sites of the most efficient esiRNAs/ERNAs on CMV RNA 3. Protein-coding regions are depicted as gray boxes. The numbers of the siRNA candidates correspond to the designations given above and below in the figures, tables and text (with the abbreviation siR or siRCV). As shown and described, a close correlation between in vitro RNA cleavage activity and in vivo antiviral activity was observed.

Table 2. esiRNA/ERNA candidates against CMV RNA 3 (SEQ ID NO: 190). 21 nt-long siRNAs from CMV RNA 3 identified in the scope of the “eRNA screens” are listed. The respective single-stranded guide and complementary passenger strands are specified. The siRNAs were named siRCV (CV for CMV) corresponding to the respective target RNA (CMV RNA 3) and the position of the 5′ end of the identified guide strand. The siRNAs were classified using standardized and stringent slicer assays as described: the percentage quantity of the respective target RNA cleaved by the esiRNAs/ERNAs in the standardized and stringent slicer assay is given. esiRNAs/ERNAs which were identified in the “eNA-screen” according to the definition (see above and FIG. 2) are highlighted in bold: AGO1-selected in black; AGO2-selected in gray. Other siRNAs that were also identified in the screen but did not meet the definition of esiRNAs/ERNAs are shown in normal font. Some of the latter siRNAs (e.g., siRCV32061, 1132) served as negative controls for infection experiments in N. benthamiana. The efficiency in protection experiments with N. benthamiana is given for siRNAs tested in planta (n.a.=not available): the proportion of asymptomatic plants 35 days after infection is given. Patent protection is accordingly preferably sought, according to the claims, for the siRNAs and variants thereof highlighted in bold (see table 6) used against CMV in the form of esiRNAs/ERNAs or sRNAs or eASOs (eNAs) derived therefrom. The respective SEQ ID NOs are given and listed separately as an “addendum to table 2.” As shown and described, a correlation between in vitro RNA cleavage activity and in vivo antiviral activity was observed. However, this was not as pronounced as in the case of the esiRNAs/ERNAs identified against CMV RNA 2 (see also text).

FIG. 6. Characterization of the protective efficiency in planta of esiRNAs/ERNAs directed against CMV RNA 3. Four- to five-week-old N. benthamiana plants were mechanically inoculated with the genomic CMV RNAs and with individual synthetic esiRNAs/ERNAs (see FIG. 3) and were monitored for the emergence of CMV-specific symptoms over a period of 28 days (dpi=days post inoculation). (A) The graph shows the percentage of asymptomatic plants over the period of the experiment. Trends with AGO1-specific esiRNAs/ERNAs are in black, and with AGO2-specific esiRNAs/ERNAs and the siR gf698 control are in gray. (B) The representative plant images illustrate the differences between asymptomatic and symptomatic plants 28 days after inoculation. The results come from three independent plant experiments. The percentage of asymptomatic plants and the number of plants used is given (n=9-15). All further controls were carried out similarly to FIG. 3. The numbers of the siRNA candidates correspond to the designations given above and below in the figures, tables and text (with the abbreviation siR or siRCV).

FIG. 7. Comparison of the target sites of CMV (Fny)-specific esiRNAs/ERNAs in RNA 2 with the corresponding potential target sites of these esiRNAs/ERNAs in the RNA 2 molecules of other CMV strains. (A) The diagram (“phylogenetic tree”) summarizes exemplary CMV strains and the specific subgroups (II, IB, IA) to which they belong. The virus Fny from subgroup IA used for the screening is highlighted with an arrow (modified according to Roossinck, 1999). (B) The overviews show exemplary AGO1-specific (top) and AGO2-specific (bottom) esiRNAs/ERNAs from CMV RNA 2 and the sequence matching thereof with the target sites in the target RNAs of different CMV strains. The “expectation” value is a measure of the mismatches between siRNA guide strand and the complementary target site. The higher the value, the lower the complementarity between siRNA and target site in the CMV RNA 2 of the CMV strain in question (https://www.zhaolab.org/psRNATarget/help#maxexpectation). The listed esiRNAs/ERNAs accordingly have very high complementarity to the genomic RNAs listed of the other CMV strains.

Table 3. CMV (Fny)-specific esiRNAs/ERNAs which are directed against RNAs 2 and 3 and are completely matched (complete matching of the corresponding target sites) to RNAs 2 and 3 of other CMV strains. (A) The table lists esiRNAs/ERNAs (in black from the AGO1 screening; gray from the AGO2 screening) which were identified from CMV (Fny) and for which the target sites match completely with potential target sites of selected CMV strains of subgroup IA (see also FIG. 7). The numbers correspond to the respective siR or siRCV in the preceding figures and tables. (A) The table lists esiRNAs/ERNAs (highlighted as above) which were identified from CMV (Fny) and for which the target sites match completely with potential target sites of selected CMV strains of subgroup IB. The esiRNAs/ERNAs identified from CMV (Fny) did not completely match with potential target sites in the RNAs 2 and 3 of selected CMV strains of subgroup II (see table 6). The numbers of the siRNA candidates correspond to the designations given above and below in the figures, tables and text (with the abbreviation siR or siRCV). The information is analogous for other sRNAs and eASOs, the sequence of which is derived from these esiRNAs/ERNAs.

Table 4. Protective potential against CMV 2 RNAs. The esiRNAs/ERNAs highlighted with a (+) have completely complementary target sites in the genomic RNA molecules of selected CMV strains. According to the data obtained with the Fny strain, particularly broad protection against CMV infection can be achieved by using the esiRNAs/ERNAs highlighted in gray and highlighted with a (+), identified from CMV RNA 2, as each of these have completely complementary target sites in five to six of the CMV strains selected for comparison. Thus, a combination of different esiRNAs/ERNAs makes it possible to provide protection against all the CMV strains of subgroups IA and IB considered herein (and probably other representatives of these subgroups) in protection experiments. Correspondingly varying the esiRNAs/ERNAs, i.e., substituting one or more of the indicated nucleotides (see table 6) makes it possible to also use the identified esiRNAs/ERNAs against strains of subgroup II. The information is analogous for other sRNAs and eASOs, the sequence of which is derived from these esiRNAs/ERNAs.

Table 5. Protective potential against CMV 3 RNAs. siRNAs highlighted with a (+) have completely complementary target sites in the selected CMV strains. Particularly broad protection against CMV infection can be achieved by using the esiRNAs/ERNAs highlighted in gray and highlighted with a (+), identified from CMV RNA 3, as each of these have completely complementary target sites in five to six of the CMV strains selected for comparison. Thus, a combination of different esiRNAs/ERNAs makes it possible to provide protection against all the CMV strains of subgroups IA and IB considered herein (and other representatives of these subgroups) in protection experiments. Correspondingly varying the esiRNAs/ERNAs, i.e., substituting one or more of the indicated nucleotides (see table 6) makes it possible to also use the identified esiRNAs/ERNAs against strains of subgroup II. The information is analogous for other sRNAs and eASOs, the sequence of which is derived from these esiRNAs/ERNAs.

Table 6. Matching of the target sites of CMV (Fny)-specific esiRNAs/ERNAs with potential target sites of these esiRNAs/ERNAs in the genomic RNAs of other CMV strains. (A) The table shows esiRNAs/ERNAs (the numbers correspond to the respective siR or siRCV in the preceding figures and tables) which were identified from CMV (Fny) and the target sites of which partially match with target sites of selected CMV strains of subgroup IA. The number of respective mismatches is given in brackets. (B) The table shows esiRNAs/ERNAs which were identified from CMV (Fny) and for which the target sites partially match with potential target sites of selected CMV strains of subgroups IB and II. The binding specificity of an sRNA to the target site of a target RNA is ensured in mismatches up to a number of 5; however, at higher melting points of the complementary RNA strands it is often still ensured even at a number of mismatches that is at least 2 nucleotides greater than that (Liu et al., 2014). As shown in table 6 except for 4 strains in which the number of mismatches is higher, all remaining strains afford potential protection in the RNA silencing method by means of the identified esiRNAs/ERNAs, even with a number of 7 mismatches to the target-site. On this basis, the claimed scope of protection (see below) is supported for eNAs that have modifications at 1-7 positions. The numbers of the candidates correspond to the designations given above and below in the figures, tables and text (with the abbreviation siR or siRCV). The information is analogous for other sRNAs and eASOs, the sequence of which is derived from these esiRNAs/ERNAs.

FIG. 8: Schematic depiction of the forms of organization of edsRNAs. edsRNAs are generated either by hybridization of two separate complementary RNA molecules (a) or by hybridization of complementary regions of one RNA molecule (b). In the case of (b), the RNA molecule contains a spacer (see text for definition). This spacer can be made smaller or completely removed (processed) by different mechanisms (e.g., splicing or endonuclease activity), as a result of which other forms of RNA hairpin molecules or similarly-constructed dsRNA molecules, like in case (a), can be formed. In the simplest case, edsRNAs as in (a) are obtained by hybridization of complementary RNA molecules generated independently of one another (see also the application examples and FIGS. 9 and 10). Black and gray boxes: pseudo (p)-siRNAs (see text for definition) with variable sequence at the 5′ and 3′ ends, respectively; (s) sense, (as) antisense. A, B . . . Y . . . Z represents 1 to n (or in the reverse order n−1) esiRNA/ERNA sequences identified from a screen with an AGO protein (A), or other AGO proteins (B . . . , Y, Z) and inserted into the edsRNA sequence; (s) sense, (as) antisense. R: Hammerhead or hepatitis delta virus (HDV) ribozyme. The ribozymes were used to generate the transcript ends via “self-splicing.” They are no longer functionally present after the generation of the respective edsRNA. Examples of edsRNAs constructed according to a) are given in FIGS. 9 and 10. Examples of the composition of cDNA constructs with which the edsRNAs designed according to a) and/or b) can be generated are given in FIG. 11.

FIG. 9. Exemplary edsRNA and control dsRNAs. The structure of an edsRNA is shown which was generated from two transcripts according to the scheme of FIG. 8A). The edsRNA contains 21 nt-long esiRNA/ERNA sequences which have been demonstrated to be effective against CMV RNA 2 in RNA silencing/RNAi processes and which afford antiviral protection in planta (numbering according to FIG. 2 and table 1). In addition, two dsRNAs are shown that are constructed conventionally, i.e., not from esiRNA/ERNA sequences, but rather from uninterrupted (continuous) regions of the target RNAs, and were used as controls. (A) Exemplary edsRNA ‘dsCMV6si21’. This consists of a 21 nt-long pseudo-siRNA at each end (symbolized by asterisks (*)) and six 21 nt-long sequences of esiRNAs/ERNAs directed against CMV RNA 2 which are active in plant AGO1/RISCs or AGO2/RISCs. Guide strands (gs) are each shown as arrows pointing in the 5′-3′ sense direction. The AGO1-specific gs are located on one RNA strand, the AGO2-specific gs are located on the other RNA strand. The example RNA shown here is blunt, i.e., there are no nucleotides overhanging at the ends. However, edsRNAs with overhanging ends (Ü) were also generated and tested (see below). The results achieved with respect to their protective efficiency were identical for both forms of the edsRNAs. The protective efficiency was also analogous with edsRNAs, which were generated from a transcript according to the scheme in FIG. 8 B) or FIG. 11 (not shown). (B) Control dsRNA 1. The dsCMV also consists of pseudo-siRNA sequences at the ends and also a double-stranded 126 nt-long section (corresponding to a length of six 21 nt-long siRNAs) from CMV RNA 2 and the complementary sequence corresponding to the section of CMV RNA 2. Important: By chance, the dsCMV also contains the sequences of two siRNAs that were identified as esiRNAs/ERNAs in the eNA screen against CMV RNA 2 (see above) (see FIG. 10). (C) Control dsRNA 2. The dsGFP also consists of pseudo-siRNA sequences at the ends and a 126 nt-long double-stranded section from the GFP mRNA (mRNA encoding green fluorescent protein) and the complementary sequence corresponding to this section of the GFP mRNA. The exact sequences of the dsRNAs shown are shown in FIG. 10.

FIG. 10. Structure/sequences of exemplary edsRNAs and the control dsRNAs used (schematically shown in FIG. 9). The sequences of two edsRNAs are shown, dsCMV6si21 and dsCMV6si22, which are constructed from pseudo-siRNA sequences (at the termini) and esiRNAs/ERNA sequences; the control dsRNAs are also shown, dsCMV and dsGFP, which are shown in FIG. 9 and are constructed from pseudo-siRNA sequences at the termini and otherwise from continuous regions of CMV RNA 2 or GFP mRNA (the sense and the antisense strands are shown in each case). The edsRNAs contain 21 nt- or 22 nt-long esiRNA/ERNA sequences for which it has been demonstrated that they are effective against CMV RNA 2 in the RNA silencing/RNAi process and afford antiviral protection in planta (FIG. 2-4). dsCMV6si21 consists of a 21 nt-long pseudo-siRNA at each end and six 21 nt-long CMV-specific esiRNAs/ERNAs (three of each active in AGO1/RISC and AGO2/RISC). The AGO1-specific guide strands (gs) are located on one RNA strand, the AGO2-specific gs are located on the other RNA strand. Since the gs of one siRNA on the dsRNA overlaps with the 2 nt 3′ overhang of the passenger strand of the respective following siRNA, some modifications were made at these sites to the two 3′-terminal nucleotides of the passenger strand, in order to obtain complete complementarity of the edsRNA. The dsCMV6si22 is constructed according to the same principle and consists of a 22 nt-long pseudo-siRNA at each end and six 22 nt-long CMV-specific esiRNAs/ERNAs (three of each active in AGO1/RISC and in AGO2/RISC). The AGO1-specific gs are located on one RNA strand, the AGO2-specific gs are located on the other RNA strand.

The different sections of the RNA are identified as follows.
dsCMV6si21 and 22 (5′-3′): bold, black—pseudo-siRNA, dark gray—siRCV21172 gs, light gray—siRCV2 1489 gs, black italic—siRCV2359 gs. gs—guide strand
dsCMV6si21 and 22 (3′-5′): bold, black—pseudo-siRNA, light gray—siRCV2380 gs, black italic—siRCV2 2041 gs, dark gray—siRCV21020 gs
The dsCMV also consists of pseudo-siRNA sequences at the ends and also a 126 nt-long double-stranded section (corresponding to a length of six 21 nt-long siRNAs) from CMV RNA 2. Important: By chance, the section selected from CMV RNA 2 also contains two siRNAs that were identified as esiRNAs/ERNAs in the eNA screen (see above) (siRCV2557 and siRCV2 540: underlined and italicized, respectively).
The dsGFP also consists of pseudo-siRNA sequences at the ends and a 126 nt-long double-stranded section of the GFP mRNA. This dsRNA contains the sequence of the control siRNA, siR gf698, (lowercase letters) used in previous experiments.
The respective SEQ ID NOs are indicated.

FIG. 11. Exemplary structure of cDNA constructs/templates by means of which edsRNAs having different structures can be generated in vitro or in vivo in various ways. The cDNAs shown contain different exemplary promoters (phage T7 RNA polymerase, Pol II and Pol I promoters (Pol II and Pol I promoters for example from Saccharomyces cerevisiae)), via which the edsRNAs of these cDNAs can be transcribed. The edsRNAs generated contain at least one pseudo-siRNA sequence and esiRNA sequences, which here correspond for example to the esiRNA sequences which were identified as directed against CMV (identical to the esiRNA/ERNA sequences in the edsRNA constructs shown in FIGS. 9 and 10). As mentioned, the esiRNA sequences used are exemplary; i.e., entirely different pseudo-siRNA sequences or esiRNA/ERNA sequences may be contained in correspondingly different, but otherwise analogously constructed cDNA constructs (see FIG. 8 and the text). In addition to the complementary pseudo-siRNA or esiRNA sequences, the cDNAs encode another spacer sequence, which here, again as an example, encodes a cellular intron (Actin 1) and can be spliced by the cellular splicing machinery or broken down by cellular RNAases. In addition, the cDNAs encode ribozymes (HH ribozyme or HDV ribozyme) which, after transcription, generate a terminus or both termini of the edsRNA by self-splicing. They also encode transcription terminators (here, for example, VSV or cellular Pol terminators) and restriction sites for cloning purposes. The respective sequences, which may also partly overlap (see text above), are identified in different ways. The respective SEQ ID NOs are indicated.

edsRNAs Generated Via T7 RNA Polymerase

Construct 1.1. edsRNA is generated as a hairpin transcript. Construct contains: promoter of T7 RNA polymerase (T7 promoter); esiRNA sequences and a pseudo-siRNA sequence that is located at one end of the esiRNA sequences; the Actin-1 intron as a spacer, the hepatitis D virus (HDV) ribozyme; the vesicular stomatitis virus (VSV) transcription terminator; restriction sites (Spe I/Xba I) for cloning.

Construct 1.2. edsRNA is generated as a hairpin transcript which is further processed. Construct contains: T7 promoter; esiRNA sequences and two pseudo-siRNA sequences that are located at both ends of the esiRNA sequences; the Actin-1 intron as a spacer; the hepatitis D virus (HDV) ribozyme; the VSV transcription terminator; restriction sites (Spe I/Xba I) for cloning.

edsRNAs Generated Via RNA Polymerase II (Pol II)

Construct 2. edsRNA is generated as a hairpin transcript which is further processed. cDNA is cloned downstream of cellular Pol II promoter, which is potentially inducible (e.g., Gall promoter of Saccharomyces cerevisiae) and termination occurs by means of a 3′-sided Pol II terminator (e.g., CYC1 terminator of Saccharomyces cerevisiae). Construct contains: hammerhead ribozyme (HH ribozyme); esiRNA sequences and a pseudo-siRNA sequence that is located at one end of the esiRNA sequences; the Actin-1 intron as a spacer, the hepatitis D virus (HDV) ribozyme; restriction sites (Hind III/Xba I) for cloning.

edsRNAs Generated Via RNA Polymerase I (Pol I)

Construct 3.1. edsRNA is generated as a hairpin transcript. Construct contains: a cellular Pol I promoter; esiRNA sequences and a pseudo-siRNA sequence that is located at one end of the esiRNA sequences; the Actin-1 intron as a spacer; the hepatitis D virus (HDV) ribozyme; a minimal Pol I terminator; restriction sites (Spe I/Xba I) for cloning.

Construct 3.2. edsRNA is generated as a hairpin transcript which is further processed. Construct contains: Pol I promoter; esiRNA sequences and two pseudo-siRNA sequences that are located on the 5′ and 3′ ends of the esiRNA sequences; the Actin-1 intron as a spacer; the HDV ribozyme; the Pol I terminator; restriction sites (Spe I/Xba I) for cloning.

FIG. 12. Processing of an edsRNA by DCLs in vitro. Radiolabeled dsCMV6si21 was added to BYL and processing by DCL4, DCL2 and DCL3, endogenously present in the extract, was monitored over a period of 24 h. Samples taken from the BYL after the indicated points in time after the dsRNA was added were separated by means of PAGE and visualized by means of autoradiography (M=21 nt siRNA as marker). The defined band pattern implies defined processing of the edsRNA by the DCL proteins. This results in a significant proportion of 21 nt siRNAs.

FIG. 13. Slicer-Assay with individual AGO1- and AGO2-specific esiRNAs/ERNAs from CMV RNA 2 and with the analogous esiRNAs/ERNAs that are generated from an edsRNA in BYL. In BYL, AGO1/and AGO2/RISC were tested with individual esiRNAs/ERNAs, with a corresponding mix of these esiRNAs/ERNAs, or esiRNAs/ERNAs were reconstituted which were processed in BYL by the DCL present there from the edsRNA ‘dsCMV6si21’. The endonucleolytic hydrolysis of the radiolabeled target RNA was detected by means of agarose gel electrophoresis and autoradiography. Asterisks (*) mark the cleavage products generated by the slicer activity of the AGO/RISC. Both the individual siRNAs and those from the edsRNA-processed siRNAs lead to efficient cleavage of the target RNA into the expected cleavage products.

FIG. 14. NGS RNA-Seq analysis of siRNAs which were generated in BYL from an edsRNA (which contains the sequences of 21 nt esiRNAs/ERNAs). The proportions of reads of 21 nt esiRNAs/ERNAs relative to the total of all 21 nt reads are given. (A) Proportion of esiRNA/ERNA guide and passenger strand reads based on the position thereof on the edsRNA dsCMV6si21, used here. (B) Proportion of guide and passenger strand reads per esiRNA/ERNA. It was possible to detect all CMV-specific esiRNAs/ERNAs. It was thus possible to demonstrate that they are processed from the corresponding edsRNA by the DCL4 contained in BYL. (C) Comparison of the detectable proportions of guide and passenger strands of CMV-specific siRNAs generated from the edsRNA. It is clear that the esiRNAs/ERNAs used were found at a high proportion (approx. 60% of the reads) and are thus preferably generated from the edsRNA by DCL4.

FIG. 15. Comparison of the protective effect of different dsRNAs in planta. Four- to five-week-old N. benthamiana plants were mechanically inoculated with different dsRNAs or single-stranded components of specific dsRNAs. The genomic, infectious CMV RNAs which, in the absence of mediated protection, trigger an infection, were administered (co-inoculated) at the same time. The percentage of asymptomatic N. benthamiana plants in each case over a period of 35 days (dpi=days post inoculation) is shown. (A) Percentage of asymptomatic plants after administration of dsCMV62i21-Ü, dsCMV6si22-Ü, dsCMV and dsGFP. (B) Representative plant images of the experiment shown in (A). The use of dsCMV6si21-Ü, consisting of six 21 nt-long CMV-specific esiRNAs/ERNAs, provides very efficient (100%) protection against CMV infection. With the analogous construct, consisting of 22 nt-long variants of the same esiRNAs/ERNAs, reduced protection (30%) of the plants against CMV infection was achieved. The conventionally constructed dsCMV (contains two esiRNA/ERNA sequences) provides even more significantly reduced protection, while dsGFP provides no protection. (C) Comparison of the protective effect of dsRNAs and single-stranded RNAs corresponding to the constituent single strands of the dsRNAs (see the text of the application example). As shown, only the double-stranded RNA consisting of a plurality of CMV-specific esiRNAs/ERNAs (dsCMV6si21-Ü) affords very effective protection against CMV infection. In contrast, the conventionally constructed dsCMV and all single-stranded components of dsRNAs do not provide any protection. This implies that the protection against virus infection provided by the edsRNA is based on its processing into esiRNAs/ERNAs by plant DCLs (see the text of the application examples).

Table 7. Sequences and activities of the esiRNAs/ERNAs identified against three different mRNA targets in the respective screenings against M. incognita. The siRNAs (again, each single-stranded guide and complementary passenger strand is listed), were identified in the manner described in the application examples using “eNA screens” and were classified as esiRNAs/ERNAs by means of slicer assays: the percentage quantity of the respective target RNA cleaved by the esiRNAs/ERNAs in the standardized and stringent slicer assays is given here. As indicated by the highlighting (bold), all listed siRNAs met the corresponding criterion of at least 25% cleavage efficiency of the originally used target RNA. The siRNAs were named siRMI (MI for M. incognita) and corresponding to the respective target RNA (SPF-Splicing factor SEQ ID NO: 191; INT-Integrase SEQ ID NO: 192; ACT-Actin 4 SEQ ID NO: 193) and the position of the 5′ end of the identified guide strand. It is also indicated whether the esiRNAs/ERNAs have a nematicidal effect in planta (yes/n.t.—not yet tested). Finally, the activity of the esiRNAs/ERNAs after treating the animals (soaking) in vivo is given: the normalized expression rate (NER) of the respective mRNAs, determined by qRT-PCR (see also FIG. 14) is given here. The values (in percent) reflect the proportion of cleaved product: e.g., in the case of siRMISPF 441, 90% of the mRNA is sliced in vivo after treatment (compared to 0% when treated with siR gf698). As shown and described, a close correlation between in vitro and in vivo RNA cleavage activity was observed. Those RNAs for which, along with variants thereof, patent protection is claimed in accordance with the claims, and which are used against M. incognita in the form of esiRNAs/ERNAs or sRNAs or eASO (eNAs) derived therefrom, are listed. The respective SEQ ID NOs are given and listed separately as an “addendum to table 7.”

FIG. 16. Silencing of the splicing factor mRNA of M. incognita by identified esiRNAs/ERNAs in vivo and in planta. A) In vivo: normalized expression rate (NER) of the splicing factor mRNA (SPF SEQ ID NO: 191), determined by qRT-PCR (the quantity of mRNA that can still be measured is shown) after incubation of J2-stage nematode larvae (J2s) for 24 hours in siRNA solution (siRMISPF 166, siRMISPF 220 and siRMISPF 441). For the gene-specific reverse transcription (RevertAid Reverse Transcriptase) to cDNA according to a standard method, approx. 500 ng of total RNA or 1/5 diluted cDNA was used as PCR template. The MI 18S rRNA (HE667742) was used as a reference gene in the quantitative RT-PCR. The normalized expression rates (NER) were calculated according to the mathematical method 2-ddCt using the mean Ct value of the reference gene. Two representative experiments with two replicates each are shown (water-water control without siRNA; siR GFP (or siR gf698)-negative control: measured mRNA quantity 100%). B) In planta/number of eggs (eggs-J2 s) in tomato roots. J2s were incubated for 24 hours in the presence of control siRNA (siR GFP or siR gf698) or test siRNA in water. The ability of the nematodes to establish infection in roots of tomato plants (two weeks old, n=15) and to complete their life cycle was analyzed by counting the eggs (eggs J2) 56 days after infection. Error bar: standard deviation of the mean (SDM). Asterisks indicate statistical differences from controls at p≤0.05 (*), p≤0.01 (**) and p≤0.001 (***), determined using a two-tailed Student's t-test. A representative experiment is shown.

FIG. 17. Silencing of Actin 4 and Integrase mRNAs in vivo. The representative experiments shown here were carried out analogously to FIG. 17. A: Normalized expression rate (NER) of Actin 4 mRNA (ACT SEQ ID NO: 193), determined by qRT-PCR (see FIG. 15) after incubation of J2-stage nematode larvae (J2s) for 24 hours in siRNA solutions (siR 154, siR 200, siR 303, siR 419, siR 433, siR 435 and siR 661). A: Normalized expression rate (NER) of integrase mRNA (INT SEQ ID NO: 192), determined by qRT-PCR (see FIG. 15) after incubation of J2-stage nematode larvae (J2s) for 24 hours in siRNA solutions (siR 135, siR 273, siR 423 and siR 444). Error bar: standard deviation of the mean (SDM). Asterisks indicate statistical differences from controls at p≤0.05 (*), p≤0.01 (**) and p≤0.001 (***), determined using a two-tailed Student's t-test.

Table 8. Sequences of the esiRNAs/ERNAs identified against different mRNA targets in the respective screenings against B. cinerea. The siRNAs (each single-stranded guide and complementary passenger strand is listed), were identified in the manner described in the application examples using “eNA screens” and were classified as esiRNAs/ERNAs by means of slicer assays: the percentage quantity of the respective target RNA cleaved by the esiRNAs/ERNAs in the standardized and stringent slicer assays is given here. The siRNAs were named siRBC (BC for B. cinerea) and corresponding to the respective target RNA (VDS-VDS51 SEQ ID NO: 194; DCTN-DCTN1 SEQ ID NO: 195; SAC-Sac1 SEQ ID NO: 196; ERG-ERG27 SEQ ID NO: 197; EF-EF2 SEQ ID NO: 198; CHS-CHS1 SEQ ID NO: 199) and the position of the 5′ end of the identified guide strand. esiRNAs/ERNAs are highlighted in bold: patent protection in accordance with the claims is preferably sought for these siRNAs highlighted in bold and variants thereof (see table 6), used against B. cinerea in the form of esiRNAs/ERNAs or sRNAs or eASOs (eNAs) derived therefrom. The respective SEQ ID NOs are given and listed separately as an “addendum to table 8.”

FIG. 18. Slicer assays with esiRNAs/ERNAs that were identified against various B. cinerea mRNAs. The siRNAs were identified by means of “eNA screens” in the manner described in the application examples, and were classified by means of standardized and stringent slicer assays.

The slicer assays were carried out as described above with AGO1 from Colletotrichum graminicula (see text). The translation reaction of C. graminicula AGO1 was carried out in the presence of the synthetic siRNA duplexes to be tested, resulting in the incorporation of the desired siRNAs into the AGO/RISC. The radiolabeled mRNAs were then added as target RNA. Total RNA was isolated from the batches and analyzed for cleavage products by means of denaturing PAGE and autoradiography. The target RNAs used (VDS represents VDS51; DCTN represents DCTN1; SAC represents Sac1; ERG represents ERG27; EF represents EF2) and the resulting cleavage products are highlighted in each case. The siRNAs are named in accordance with table 8. For the comparison, siRNAs (257, 470, 653, 808) determined by in silico prognosis (https://www.zhaolab.org/pssRNAit/) were used in each case in the assay. The Slicer assay was carried out without siRNA (−) as a control. As a further control, originally used dsRNAs (ds) and pools of siRNAs (“pool”) were applied to the gel.

FIG. 19. Inhibition of the growth of B. cinerea by topical application of esiRNAs/ERNAs that are directed against defined target RNAs. (A) Bar charts representing the size of B. cinerea lesions which developed on leaves of Arabidopsis thaliana (wild-type; Col-0). The plants were inoculated with suspensions of B. cinerea spores and various combinations of siRNAs: i) a mixture of six esiRNAs/ERNAs directed against Erg27 mRNA (siRBCERG); ii) a mixture of four esiRNAs/ERNAs directed against Erg27 mRNA and two esiRNA/ERNAs directed against SacI mRNA and one esiRNA/ERNA directed against Ef2 mRNA (siRBCMIX); and iii) one siRNA directed against GFP-mRNA (siR gf698 or siR GFP). Twelve plants were used for each treatment, and three leaves (leaves 8, 9 and 10) were inoculated per plant. A drop of the suspensions containing the spores and 400 ng (siR gf698 or siR GFP), 2400 ng (siRBCERG) or 2800 ng (siRBCMIX or siR GFP) of the RNAs was added to each leaf. Suspensions without RNAs (“water”) were used as fungal growth controls. The lesion area on the leaves (see examples in the lower panel) was determined 3 days after inoculation (dpi) using ImageJ software, and the sizes were divided into five categories: i) >50 mm² (+++), ii) 20-50 mm² (++), iii) 10-20 mm² (+), iv) 1-10 mm² (+/−), and v) 0 mm² (−). The different categories are given in percentages. Representative images of leaves with lesions of the different categories (lower panel). (B) qRT-PCR determination of the Erg27 mRNA level in vivo (during the fungal infection process) 3 dpi, total RNA was extracted from the fungal lesions of the leaves and cDNA was generated using a standard protocol. For the subsequent PCR amplification, two primer sets were used, covering the regions targeted by the various esiRNA/ERNAs (upper region). The level of Erg27 mRNA was normalized to the endogenous level of B. cinerea Actin mRNA (“housekeeping”). The normalized expression rate (NER; see also previous figures) is given. The bars represent the mean of four biological replicates (each containing six lesions from independent leaves), the error bars and the SDM (standard deviation of the mean; lower region). Statistically significant differences from the water control (“Water”) were determined using a two-tailed Student's t-test: *p≤0.05, *p≤0.01, ***p≤0.001.

### APPLICATION EXAMPLES

### Application Example 1: Nucleic Acid Active Agents Against Cucumber Mosaic Virus, CMV

The “eNA screening” method was used, first of all in this form, on two RNA segments of the CMV genome (Fny strain) as target RNAs: RNA 2 (SEQ ID NO: 189) encodes the 2a protein, and the subgenomic RNA 4A, which in turn encodes the viral suppressor of RNA silencing (VSR), 2b. RNA 3 (SEQ ID NO: 190) encodes the 3a protein, and the subgenomic RNA 4, which in turn encodes the capsid protein (CP) (FIG. 2 D and FIG. 5). The screening was carried out with both AGO1 (L version; Gursinsky et al., 2015) and AGO2 from Nicotiana benthamiana (Nb). The target RNAs were used in double-stranded form in the screens.

Thus, a series of siRNAs, which were classified as esiRNAs/ERNAs, were identified against CMV RNA 2 with NbAGO1L and NbAGO2 (FIG. 2; table 1). As described above, the classification as esiRNAs/ERNAs took place in the final step of the “eNA screen” in standardized and stringent slicer assays (protocol modified from WO 2019/001602; WO 2022/200407 and Gago-Zachert et al., 2019; see also general description of the method above): to form the RISC, 0.5 μmol of the mRNA of the AGO protein to be used was translated in a reaction solution (Gago-Zachert et al., 2019) containing 50% (v/v) BYL with defined protein quantity and translation activity, in the presence of 10-100 nM of the synthetic siRNA to be characterized and a 10-fold excess (0.1-1 μM) of a competitor siRNA (siR gf698). The quantity used of the siRNA to be tested was adjusted to the activity of the AGO protein in question with the siRNA siR gf698 on the GFP mRNA target thereof. siR gf698 meets the criteria of an esiRNA/ERNA on the GFP mRNA (Schuck et al., 2013). After an incubation time of 2.5 h at 25° C., 3.4 μmol of a nonspecific mRNA (encoding the Firefly luciferase protein) were added per batch as a further competitor RNA, and also 10 fmol of the radiolabeled target RNA were added, and the reaction batches were incubated again for 15 min at 25° C. During this incubation, the target RNA is potentially cleaved by the AGO/RISC formed. The further conditions and the analysis of the cleavage reaction was in accordance with Gago-Zachert et al., 2019: after gel electrophoresis of the extracted RNA, the remaining quantity of target RNA or the resulting cleavage products compared to a control reaction (carried out without siRNA) were quantified by measuring the band intensities (ImageQuantTL or ImageJ). Classification as esiRNA/ERNAs was made on the basis of the cleavage activity (slicer activity) measured in this way, of the respective RISC formed with this siRNA, on the target RNA (see also the tables): if at least 25% of the quantity of target RNA originally used in the assay is endonucleolytically converted to cleavage products under the standardized and stringent (competitive) conditions, this siRNA is designated as effective, i.e., as esiRNA/ERNA. The setting of 25% cleavage efficiency as the threshold value was chosen on account of earlier data (Gago-Zachert et al. 2019) and also the data obtained here, which show that esiRNAs/ERNAs that have this characteristic in vitro have a clearly measurable antipathogenic effect in vivo compared to control siRNAs (see below). esiRNAs/ERNAs which, with AGO1, are particularly cleavage-active on CMV RNA 2, were characterized on this basis. These were, for example, siRCV2359, siRCV21172 and siRCV21489 (the candidates are named with “siR,” “CV” for CMV, the respective RNA and the position of the viral (+)RNA to which the 5′ nucleotide of the siRNA guide strand is complementary). siRCV2149, siRCV2 186, siRCV21613, siRCV21982, siRCV22441, siRCV22562 and siRCV22727 also had high cleavage activity. The functional in vitro data with these siRNAs are shown in FIG. 2; the sequences of these siRNAs are summarized in table 1 (in the addendum to table 1 with the respective SEQ ID NO).

Analogously, siRCV2 380, siRCV2 1020 and siRCV2 2041 were identified as particularly cleavage-active RNA active agents in the RISC complex together with AGO2. siRCV2407, siRCV2449, siRCV2540, siRCV2557, siRCV21054 and siRCV21248 also had a high cleavage rate (FIG. 2; table 1). The binding sites of the particularly cleavage-active (most efficient) siRNAs located on the RNA 2 are schematically illustrated in FIG. 2 C.

By way of example, the esiRNAs/ERNAs siRCV21020, 1172, 359, 1489, 380 and 2041 identified in vitro were used in protection experiments in planta. For this purpose, a statistically representative number of N. benthamiana plants (n=12-15) were co-inoculated using carborundum by means of a standard protocol (“rub-in”) with 150 μmol (˜1 μg) of the siRNA to be tested (synthetically obtained from a company) and 20 fmol each of the genomic CMV RNAs (1-3; produced by in vitro transcription from “infectious cDNAs” (obtained from Prof. Fernando García-Arenal Rodríguez (Polytechnic University of Madrid) and Prof. John Carr (University of Cambridge); Rizzo and Palukaitis 1990) per plant (RNAs dissolved in 15 mM KH2PO4, 25 mM glycine solution). Infection with the CMV-RNAs was carried out such that 100% of the respective quantity of RNAs, when inoculated without additives in N. benthamiana, led to an infection with a significant development of symptoms (what is referred to as “maximal challenge”). The nonspecific siR gf698, or siRNAs having no or only slight cleavage activity on the target RNA in the screen carried out beforehand (siRCV21844 and siRCV22634), were used as controls. The plants were examined for symptom development for 35 days (dpi; days post infection). FIG. 3A shows representative images of plants treated in the manner mentioned. FIG. 3 B shows the overall trends across multiple experiments (three biological replicates). It was clear that siRCV2 359 and siRCV21489 each achieve 93% protection against a CMV infection and siRCV21020 and siRCV21172 each achieve 100% protection against a CMV infection: in other words, all, or the vast majority of, plants treated in this way remain asymptomatic in the maximal challenge used. 60% protection against a CMV infection was achieved with siRCV2380 and siRCV2 2041: in other words, 40% of these plants developed symptoms during the challenge. The various negative control siRNAs provided little or no protection: only 0-7% of the plants treated in this way remained asymptomatic during the challenge. It was also possible to confirm this protective effect through the fact that, in plants that were identified “by eye” as asymptomatic 35 dpi after treatment with the respective protective esiRNAs/ERNAs, it was no longer possible to detect genomic CMV RNA by RT-PCT (standard procedure) (FIG. 3 C).

It was also possible to achieve plant protection against a CMV infection using 22 nt versions of the respective esiRNAs/ERNAs. This is shown by way of example in FIG. 4: in in vitro slicer assays, 22 nt esiRNA/ERNA versions exhibited similar, or occasionally slightly lower, slicer activity on the target RNA in the respective AGO/RISC. A similar trend was observed in plant protection experiments: either the protection with 22 nt siRNAs was comparably good, or it was slightly reduced compared to the situation with 21 nt esiRNAs/ERNAs (shown by way of example in FIG. 4 with siRCV2359 and siRCV21020). Slicer assays and protection experiments with 24 nt-long versions of the respective esiRNAs/ERNAs showed similar results to the experiments carried out with the 22 nt esiRNAs/ERNAs (not shown).

Using the same method, esiRNAs/ERNAs against CMV RNA 3 were identified; use was again made here of NbAGO1L and NbAGO2. Here, too, a series of siRNAs were identified which, with AGO1, induce very high (siRCV3239, siRCV3507, siRCV3985) or high (siRCV3 151, siRCV3988, siRCV31098) slicer activity against the CMV target RNA 3 and which, with AGO2, induce very high (siRCV3593, siRCV31019, siRCV31569) or high (siRCV3358, siRCV3478, siRCV3496, siRCV3592, siRCV3733, siRCV31394) slicer activity against the CMV target RNA 3 (FIG. 5A, B). The position of the most efficient siRNA guide strands in the slicer assay on the RNA 3 is shown in FIG. 5 C. Table 2 summarizes the results from the screen of esiRNAs/ERNAs (in the addendum to table 2 with the respective SEQ ID NO) against CMV RNA 3.

In comparison, the esiRNAs/ERNAs characterized against CMV RNA 2 tended to exhibit higher slicer activity than the esiRNAs/ERNAs identified against RNA 3 (see tables 1 and 2). The reasons for this could be a slightly higher stability or fundamentally more compact structure of CMV RNA 3 (lower accessibility of a-sites) compared to the situation for CMV RNA 2 (data not shown).

In protection experiments in plants, the 21 nt and 22 nt esiRNAs/ERNAs characterized against CMV RNA 3 exhibited effective protection against the virus. As expected from the in vitro experiments, this was somewhat lower than was the case with the esiRNA/ERNA active agents against CMV RNA 2 (FIG. 6). Slicer assays and protection experiments with 24 nt-long versions of the respective esiRNAs/ERNAs showed similar results to the experiments carried out with the 22 nt esiRNAs/ERNAs (not shown).

Sequence comparisons revealed that many of the esiRNAs/ERNAs identified here from the CMV Fny strain are also effective on the analogous RNA segments of other CMV strains, because there is complete complementarity between the sequences of the respective guide strands of these esiRNAs/ERNAs and the respective target sites on the viral RNAs. FIG. 7 shows, as an example, the matches of the sequences to the complementary target sites on the RNA 2 genome segments of CMV for six esiRNAs/ERNAs (modified according to Roossinck, 1999). The figure also shows calculated expectation rates for RNA-RNA mismatches (missing base pairing at one or more positions in the nucleic acid when the siRNA guide strand binds to the target site). Based on these data, table 3 provides a complete overview of the identified esiRNAs/ERNAs, the guide strands of which bind with complete complementarity, i.e., without mismatches, to the target sites in RNAs 2 and 3 of the respective CMV strains. As is clear from the table, these esiRNAs/ERNAs, used either individually or in combination, are thus protective against practically all CMV strains of groups IA and IB. This is clarified in tables 4 and 5.

As is clear from tables 3-5, none of the identified esiRNAs/ERNAs bind without mismatch to the corresponding target sites in RNAs 2 and 3 of CMV strains of subgroup II. As explained above and previously described in an application (WO 2022/200407), esiRNAs/ERNAs indicate what are referred to as a-sites, i.e., regions containing the target sites of these esiRNAs/ERNAs in target RNAs of complex structure which, despite this structuring, are accessible to RISC or other endonuclease-containing cellular complexes (WO 2022/200407). Accordingly, by adapting the sequences of the esiRNAs/ERNAs identified here to the target sites in the target RNAs, i.e., by avoiding mismatches, silencing of the genomic RNAs 2 and 3 of the strains of subgroup II can nevertheless take place. This is summarized in table 6 which shows the number of mismatches of the guide strands of the identified esiRNAs/ERNAs upon binding to the respective target sites in RNAs 2 and 3 of the various CMV strain subgroups. The table shows all forms of mismatches. These can involve what is referred to as the seed region of the siRNAs, the most important region of interaction of an siRNA with the target RNA during the AGO/RISC-mediated silencing process (Jackson und Linsley, 2010), and also the 5′ end of the RNA which has been proven not to be involved in the binding of the siRNA. The binding specificity of an sRNA to the target site of a target RNA is ensured in mismatches up to a number of 5; however, at higher melting points of the complementary RNA strands it is often still ensured even at a number of mismatches that is at least 2 nucleotides greater than that (Liu et al., 2014). As shown in table 6 except for 4 strains in which the number of mismatches is higher, all remaining strains afford potential protection in the RNA silencing method by means of the identified esiRNAs/ERNAs, even with a number of 7 mismatches to the target-site.

To summarize this data, the population of esiRNAs/ERNAs identified here can either be used in unmodified form (as is the case for most of the members of subgroups 1a and 1b) or in a form varied at 1 to a maximum of 7 nucleotides (table 6) for the purpose of protection against most CMV strains. As is clear from the table, according to the invention, practically 95% of all CMV strains compared can be targeted by the mapped esiRNAs/ERNAs, even with a range of variations at 7 positions. In the same context, it is important to note that the esiRNAs/ERNAs can either be used individually or, much more effectively, as a combination (mix) in RNAi methods (see also below). In this way, even in the case of existing mismatches of individual esiRNAs/ERNAs, escape via antigenic drifts or shifts can be particularly effectively prevented. The analogous finding applies to other sRNAs and eASOs (collectively referred to as eNAs), the sequences of which could be derived from the corresponding esiRNAs/ERNAs.

Summary of application example 1: the stated problems were solved. According to the invention, esiRNA/ERNA active agents or eNA active agents derived therefrom were identified which have an antiviral effect against CMV and can be used in plant protection against CMV. The identified e NAs can be used either in unmodified form or in a form modified at 1 to 7 nucleotide positions for the purpose of protection against 95% of known CMV variants. The eNAs can either be used in plant protection individually or, even more effectively, as a combination (mix) in RNA silencing approaches against CMV.

### Application Example 2: Double-Stranded RNA Active Agents that Contain

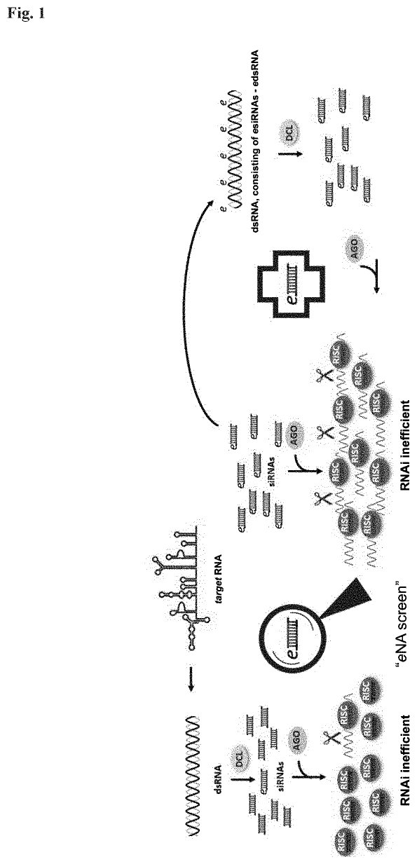

esiRNA/ERNA sequences or sequences of other related sRNAs. The object was to construct double-stranded RNAs, what are referred to as edsRNAs, which are optimized for the practical application of esiRNAs/ERNAs or related sRNAs in the RNA-silencing/RNAi process. edsRNAs should accordingly contain the sequences of a plurality of esiRNAs/ERNAs and/or sRNAs derived therefrom (see FIG. 1). When used in the target organism, the edsRNAs should be processed by the DCLs/Dicer present therein to give a high proportion of the originally constituent esiRNAs/ERNAs or sRNAs, and these should then be active in the corresponding RISC against target RNAs. In order to generate a maximally effective silencing response by these edsRNAs against one or even a plurality of target RNAs, these edsRNAs should, as a further property, consist of sequences of esiRNAs/ERNAs or related sRNAs which can be active in RNA silencing/RNAi in various AGO proteins and thus various RISCs.

According to the invention, the design of the edsRNAs was based on a hitherto incompletely proven hypothesis. This hypothesis assumes that DCLs/Dicer can be active at both ends (termini) of a dsRNA. The hypothesis also includes it being possible for DCLs/Dicer to be forced into “synchronized processing” by the presence of what are referred to as “pseudo-siRNA sequences” at the ends of the dsRNA. A pseudo-siRNA sequence within the meaning of the invention is therefore a double-stranded ribonucleotide sequence of any composition which, according to the planned processing of the respective edsRNA by DCLs/Dicer, is 21, 22, 23 or 24 nt long. Pseudo-siRNA sequences should accordingly be located at the termini of the double-stranded RNA and their presence should force the DCLs/Dicer active on this dsRNA into synchronized processing. Synchronized processing means that, depending on the position and length of the pseudo-siRNA sequences and accordingly on the activity of the DCLs/Dicer involved, the endonucleolytic cleavage of the dsRNA occurs in such a way that the sRNAs preferably generated in the process have the same length as the pseudo-siRNAs. For example, if the pseudo-siRNA sequences are 21 nt long and DCL4 is the processing enzyme (DCL4 preferably generates 21 nt siRNAs from a dsRNA), then 21 nt sRNAs are preferably generated from an edsRNA constructed in this way, and they are generated in the order in which they appear in the sequence after the pseudo-siRNAs in the edsRNA. DCL4 acts processively: starting from the pseudo-siRNA sequences at the termini of the edsRNA, the enzyme makes endonucleolytic cuts one after the other. If an edsRNA is constructed such that, e.g., 21 nt-long esiRNA/ERNA sequences directly follow 21 nt-long pseudo-siRNA sequences (see FIG. 8-11), 21 nt-long esiRNAs/ERNAs should then be formed preferentially.

Pseudo-siRNA sequences may also contain elements that are important for successful transcription and processing of the respective edsRNAs: these can be regions of the respective transcription promoter or terminator; but they can also be parts of a ribozyme which generates the correct 5′ or 3′ end of the respective RNAs (see FIGS. 8 and 11). The latter applies, for example, to HH ribozymes; an example was used in this application example. Here, the pseudo-siRNA sequence contains a defined number of nucleotides that are complementary to the 5′ end of the ribozyme and can form the helix I thereof and thus the functional structure thereof through hybridization (FIG. 11).

The basic principle of the construction of edsRNAs designed in this way is thus presented as follows (FIG. 8 and following):
The sequence contains at least one pseudo-siRNA sequence which, as stated, enables synchronized processing by Dicer/DCLs. The sequence of the edsRNAs also contains a number of sequences, “in a row” from 5′ to 3′, of esiRNAs/ERNAs or of other sRNAs, for example miRNAs, derived from esiRNAs/ERNAs, described in FIG. 8 as 1-n in the sense(s) direction and n−1 in the antisense (as) direction. These sequences can originate from various “eNA screens,” and the esiRNA/ERNAs or sRNAs derived therefrom can accordingly be active in different AGO/RISC (described in FIG. 8 as A-Z). In the optimal case, the esiRNA/ERNAs or sRNAs derived therefrom, which constitute an edsRNA, can also be directed against different target RNAs from one or more different organisms. n in FIG. 8 is a number between 2 and infinity, preferably between 2 and 100, or is as described above for group d.
An edsRNA can be generated in two ways: from two complementary RNA molecules or from one transcribed RNA molecule containing two complementary sections (FIG. 8). In the latter case, the two complementary sections of the transcribed RNA are connected to each other by a spacer and form a hairpin. The spacer is a sequence of any composition having a minimum length of 4 nucleotides (the definition, and that for hairpins, is given above) which however can contain functional regions such as ribozymes, transcription promoters or transcription terminators, transport signals or splice sites for the specific purpose of edsRNA construction. In this way, in addition to the function of connecting the two complementary single-stranded components of a dsRNA, the spacer sequence also serves further purposes: if it contains, for example, splice signals, the spacer can be truncated by the cell's splicing machinery in the course of in vivo RNA expression. Since, in contrast to the double-stranded RNA regions, the spacer is sensitive to ribonucleases (such as the single-strand-specific RNases T1 or A), this sequence can also be completely removed by these RNases (see also FIG. 8).
The transcription of RNAs can take place in vitro or in vivo. The double strand is obtained by hybridization of the complementary RNA strands (FIG. 8). Transcription can take place via a wide variety of promoters (see for example FIG. 11). Transcription termination can take place via any type of transcription terminators (see for example FIG. 11).
Depending on the manner of generation, the termini of the edsRNAs are either blunt or they contain an overhang (−Ü). They can be generated in various ways, for example by “run-off transcription” or by terminating the polymerases in question using the activity of ribozymes (e.g., HH or HDV ribozymes as used in this application example) via self-splicing.
The sequences of both strands are designed in such a way that each of the authentic sRNA guide and passenger strand sequences are generated during processing by DCLs. Processing, which predominantly leads to the generation of the constituent esiRNAs/ERNAs or sRNAs derived therefrom, is ensured by the presence of the pseudo-siRNA sequences and the associated “synchronized processing” by the DCLs/Dicer (see above and FIG. 8-14).

The hypothesis of “synchronized processing” and therefore the functionality of various edsRNA constructs constructed according to this principle was verified and confirmed according to the invention, i.e., using the iterative trial and error principle. Examples of the composition of simply designed edsRNAs that can be generated via hybridization between two complementary RNA molecules are given in FIGS. 9 and 10 (SEQ ID NO: 181 and 185, and 182 and 186). Such edsRNAs can be generated by in vitro transcription of conventionally constructed cDNAs consisting of a promoter, the coding sequence and a terminator/run-off. Examples according to the invention for the composition of edsRNAs which can be generated both in vitro and in vivo are given via the depiction of the underlying cDNA constructs (SEQ ID NOs: 200, 201, 201, 203 and 204) in FIG. 11. The latter edsRNAs all contain a spacer which can remain in place for the use or, as described above, can be truncated or broken down.

FIGS. 12-15 demonstrate the functionality of edsRNAs designed in this way. This is shown by way of example using the example of the edsRNA ‘dsCMV6si21’ (FIGS. 9, 10 and 15; SEQ ID NOs 181 and 185). This edsRNA contains six different, 21 nt-long esiRNAs/ERNA sequences directed against CMV RNA 2. Three of these esiRNAs/ERNAs were shown in application example 1 to be active in plant AGO1/RISC against CMV RNA 2; another three of these esiRNAs/ERNAs have previously been shown to be active in plant AGO2/RISC against CMV RNA 2. In this case, the functionality of the edsRNA was independent of whether it had been generated in vitro or in vivo (FIG. 11 and not shown) or whether this RNA was blunt or had overhanging ends (−Ü). The functionality of the edsRNA was also independent of whether it had been constructed from 21, 22 or 24 nt-long pseudo-siRNAs and esiRNAs/ERNAs.

First, it was demonstrated that, in BYL, which has been shown to contain DCLs 4, 2 and 3 in active form (Schuck et al., 2013), the edsRNA is processed to give 21 nt-, 22 nt- and 24 nt-long siRNAs, respectively. Thus, the fundamental processability of the edsRNA constructs designed by Dicer/DCL was demonstrated (FIG. 12).

By using the edsRNA in in vitro slicer assays with the target RNA, it was possible to demonstrate that the cleavage products to be expected by the activity of the esiRNAs/ERNAs obtained are generated from the target RNA (as an example CMV RNA 2) (FIG. 13). This was also confirmed in a follow-up experiment. There, the siRNAs processed from an edsRNA, dsCMV6si21, in BYL (by the DCLs present and active therein) were determined by NGS (RNA-seq): it is clear that high proportions of both the pseudo-siRNAs and the esiRNAs/ERNAs (identifiable on the detectable guide or passenger strands) are processed from the edsRNA (FIG. 14A+B; 21 nt reads are shown in each case). The hypothesis put forward according to the invention, which led to the determined structure of edsRNAs, therefore proved to be correct: from dsRNAs constructed in this way, DCL4 preferably generates the 21 nt pseudo-siRNAs and esiRNAs/ERNAs: in total, these account for approx. 60% of all the 21 nt siRNAs generated (FIG. 14 C). According to the invention, it was shown that processing by the DCLs takes place from both ends of the dsRNA in the form of a “synchronized processing.”

Finally, plant protection experiments demonstrated the high efficacy (protectiveness) of using edsRNAs against a CMV challenge (infection with a lethal concentration of CMV) and thus ultimately demonstrated the functionality of the edsRNAs (FIG. 15).

Several important aspects became clear from the exemplary experiments shown:
Firstly, it was shown that an edsRNA (dsCMV6si21 or dsCMV621-Ü) constructed from 21 nt esiRNA/ERNAs is 100% protectively effective compared to a nonspecific dsRNA (control dsRNA 2, dsGFP; consisting of the sequence of the analogous length from the GFP mRNA and the RNA complementary thereto). In other words, all of the plants treated with the edsRNA remained asymptomatic in the challenge experiment with CMV, compared to the dsRNA constructed nonspecifically (FIG. 15).
Compared to a conventionally* constructed dsRNA containing a ds sequence from CMV RNA 2 which is the analogous length as dsCMV6si21 (control dsRNA 1, dsCMV; consisting of a region of CMV RNA2 of the same length and the RNA complementary thereto; see FIGS. 9 and 10), edsRNAs had a significantly more efficient antiviral efficacy:
*“Conventional dsRNAs” means that one strand of these RNAs consists of an exact copy of the targeted target RNAs and that this strand is then hybridized with a complementary RNA strand: dsRNAs constructed according to this principle are currently used in RNAi-mediated plant protection.

Although the conventionally constructed dsCMV (control dsRNA 1) exhibited a certain degree of protection compared to dsGFP (control dsRNA 2), this protection did not last over the entire test period of 35 dpi. In comparison, as mentioned, the edsRNA constructed from esiRNA/ERNAs had protection that remained at 100% over the entire test period (FIG. 15). It is important to note here that the dsCMV used as a control also contained two of the sequences characterized here previously as esiRNAs/ERNAs (shown in FIG. 9). Thus, this control was a very good reflection of the situation of a treatment with a conventional dsRNA in which only a few, if any, esiRNA/ERNA sequences are present (schematically shown in FIG. 1).
edsRNAs that generated 21 nt esiRNAs/ERNAs were the most efficiently protective against CMV; edsRNAs that produced the same esiRNAs/ERNAs but as 22 nt versions were less protective: in contrast to the 21 nt edsRNA, the 22 nt edsRNA did not exhibit 100% protection over the entire test period. Nevertheless, the protection provided by the edsRNA generating the 22 nt esiRNAs/ERNAs was significantly more pronounced in comparison to the conventionally constructed dsCMV (FIG. 15). The same applies to an edsRNA generating 24 nt esiRNAs/ERNAs (not shown).
When the 170 nt-long single-stranded components of the dsRNAs (dsCMV6si21 or dsCMV621-Ü and dsCMV) were tested in protection experiments, they were found not to provide any protection (FIG. 15 C). This experiment, conducted as a further control, demonstrated that edsRNAs used are actually exclusively active in the double-stranded form. The observed protective effect must therefore be based on the (synchronized) processing of the edsRNAs by the plant DCLs in vivo to give esiRNA/ERNAs. In other words, it was possible to rule out that the 21, 22 or 24 nt-long sections from the single-stranded components of the dsRNAs associated by chance in the course of the experiment with the target RNAs and thereby triggered AGO/RISC-mediated slicing and thus protection.

Summary of application example 2: according to the invention, entirely newly designed and constructed edsRNA active agents were developed that can be simply produced by in vitro or in vivo transcription and from which significant quantities of the various constituent sRNAs are generated by processing by Dicer/DCIs. edsRNAs constructed in this way can be used as efficient antipathogenic active agents.

### Application Example 3: Nucleic Acid Active Agents Against Meloidogyne incognita

esiRNAs/ERNAs against three mRNAs of Meloidogyne incognita target genes (SEQ ID NOs: 191 to 193) were identified in BYL using the method described with the AGO2 protein of Nicotiana benthamiana (Nb): these target genes encode the proteins “splicing factor,” “Actin-4” and “integrase.” Alongside other factors, it was assumed that each of these proteins has an essential function in the life cycle of Meloidogyne incognita (see also above).

The respective gene or target RNA sequences (see appendix) were established as follows. In the case of Actin-4: the sequence was determined using the accession number of Actin-4 for C. elegans in https://wormbase.org//#012-34-5. A sequence comparison (Blast N) was thus carried out in the database https://meloidogyne.inrae.fr/. The sequence of the predicted cDNA in M. incognita was determined in this way. This was in turn confirmed by sequence comparison of the derived protein sequence (Blast P) in the NCBI database (National Center for Biotechnology Information) and cloned. In the case of Splicing factor and Integrase: EST (expressed sequence tag) sequences of M. incognita (AW828516 and AW871671, respectively) from the NCBI database were used here. These were used for a Blast N in the database https://meloidogyne.inrae.fr/. After the predicted cDNA sequences were found, a Blast P was carried out with the derived protein sequences, making it possible to confirm that they are homologous to the splicing factor and integrase sequences of C. elegans. These cDNAs were accordingly cloned.

The esiRNAs/ERNAs summarized in table 7 (SEQ ID NO in addendum to table 7) were identified and classified in accordance with the above-defined characteristics. Naming is devised as follows: “siR,” “MI” for M. incognita, the respective mRNA (SPF, Splicing factor; INT, Integrase; ACT, Actin 4) and the position in the RNA to which the 5′ nucleotide of the siRNA guide strand is complementary. They were validated in various ways (see FIGS. 16 and 17).
Standardized slicer assays according to the protocol described above. The esiRNAs/ERNAs and edsRNAs derived therefrom (FIGS. 8 and 11) were validated in slicer assays with NbAGO2 with respect to their cleavage (slicer) activity on the respective target RNAs in vitro. All 14 of the siRNAs identified using the “eNA screen” exhibited induced hydrolysis of the target RNAs which was more than 25% of the quantity of these target RNAs originally used (summarized in table 7).
In vivo. In addition, the cleavage efficiency of the respective mRNA after the siRNAs have been taken up was tested in vivo (i.e., in animals). To this end, J2 M. incognita animals were obtained, and 10,000 of them were incubated (soaked) for 24 hours in 40 μl of water including 50-200 ng/μl RNA in order for them to take up the RNAs. The siRNA siR gf698 (siR GFP) was used as a negative control. The nematodes were then digested using a standard method, the total RNA was extracted and the cleavage of the respective target mRNA was investigated using corresponding primers and quantitative real-time PCR (qRT-PCR). For the gene-specific reverse transcription (RevertAid Reverse Transcriptase, ThermoFisher) to cDNA, also according to a standard method, approx. 500 ng of the total RNA or 1/5 diluted cDNA was used as PCR template. The reference gene used was the M. incognita GAPDH gene (Minc3s07075g40689). The normalized expression rates (NER), which reflect the measurable quantity of target mRNA originally used in the assay and remaining after cleavage (see also FIG. 14), were calculated according to the mathematical method 2-ddCt using the mean Ct value of the reference gene.
In vivo/in planta. Finally, the ability of the esiRNAs/ERNAs to suppress the complete nematode life cycle in planta was tested. In a first form of the experiments, J2 animals were placed (“soaked”) in water-soluble RNAs or control RNA for 24 hours, as described above. Subsequently, two-week-old tomato plants were infected with the nematodes treated in this way according to a standard protocol (500 animals per plant) and it was investigated whether this treatment led to a reduction in egg laying after infection or to reduced pathogenesis (in particular gall formation) of the infected plants.

In a second form of these experiments, infection studies were carried out on fresh seedlings. To this end, the surfaces of tomato seeds were sterilized by soaking in 70% ethanol. After removal of the ethanol, for further treatment, the seeds were treated with a solution containing 30% (v/v) NaOCl and 0.02% Triton X-100 and were incubated for 20-30 minutes. They were then washed with sterile distilled water and placed in Petri dishes containing approximately 6 mm of 0.6% Phytagel, pH 6.4, in 1/4 MS (Murashige and Skoog medium) including 0.5% sucrose. Ten seeds were randomly distributed in each Petri dish, the dishes were then sealed with Nescofilm and kept at 28° C. with a 16-hour light/8-hour dark cycle. Seven to ten days after sowing, each seedling was inoculated with 100-200 sterile and soaked J2s. To this end, the surface of the J2 animals was sterilized for 5 minutes in a solution containing 0.004% mercury chloride, 0.004% sodium azide and 0.002% Triton X-100, washed with sterile water and suspended in agarose (0.1%). The soaking was carried out as described above. The inoculated seedlings were grown under the conditions described above. The infection process was monitored by examining gall formation three weeks after inoculation.

In the tests, the esiRNAs/ERNAs listed in table 7 had a significant silencing or nematicidal effect (see FIGS. 16 and 17).

Summary of application example 3: the stated problems were solved. According to the invention, esiRNA/ERNA active agents or eNA active agents derived therefrom were identified which have a nematicidal effect against M. incognita and can be used in plant protection against M. incognita. The identified eNAs can be used either in unmodified form or in a form modified at 1 to 7 nucleotides for the purpose of protection against all known M. incognita variants. The eNAs can either be used in plant protection individually or, much more effectively, as a combination (mix) in RNA silencing approaches against M. incognita. It was also possible to use edsRNAs containing the sequences of identified esiRNAs/ERNAs or other sRNAs derived therefrom in plant protection against M. incognita.

### Application Example 4: Nucleic Acid Active Agents Against Botrytis cinerea

esiRNAs/ERNAs against various mRNAs of B. cinerea target genes were identified and classified in BYL using the method described with the AGO1 protein of Colletotrichum graminicula (C. graminicula, like B. cinerea, is a plant-pathogenic fungus). Three of the target RNAs (SEQ ID NOs: 197, 199, 198) were selected because of the fact that each of the encoded proteins, “Cytochrome P450-Monooxygenase” (Erg27), “Chitin Synthase 1” (CHS1) and “Elongation factor 2” (EF2), were already known as target molecules of various fungicides (see the introduction regarding B. cinerea). Three more target RNAs (SEQ ID NOs: 194, 195, 196) were selected because of the fact that each of the encoded proteins, “Vacuolar protein sorting 51” (VPS51), “Dynactin” (DCTN1) and “Suppressor of actin” (SAC1), are involved in vesicular transport path in the fungus and are essential virulence factors of B. cinerea during interaction with plant hosts.

The cDNAs for these genes were generated and cloned from an mRNA preparation. The mRNA preparation was prepared from B. cinerea-infected Arabidopsis thaliana plants according to a standard method.

The esiRNAs/ERNAs summarized and highlighted in table 8 were identified (SEQ ID NO in “addendum to table 8”). Naming of the candidates is devised as follows: “siR,” “BC” for B. cinerea, the respective RNA (ERG-Erg 27; CHS-Chitin-Synthase 1; EF
Elongation factor 2; VPS-Vacuolar protein sorting 51; DCTN-Dynactin1; SAC-Suppressor of actin) and the position in the RNA to which the 5′ nucleotide of the siRNA guide strand is complementary. They were validated as follows.
   Standardized slicer assays according to the protocol described above. To this end, the RNAs were tested in vitro with C. graminicula AGO1 for the cleavage (slicer) activity of the respective targetRNAs: 24 of a total of 30 siRNAs identified using the “eNA screen” exhibited induced hydrolysis of the respective target RNA, which was more than 25% of the quantity of these target RNAs originally used (FIG. 18 and table 8).
   In vitro fungal growth experiments. B. cinerea B05.10 was cultured on fruit. After the fungus had been able to sporulate for five days, the spores were collected and diluted in the corresponding medium to a final concentration of 1×10⁵ spores/ml (standard method). Germination of the spores was then initiated by adding phosphate. To enable this to happen uniformly, 10 μl of 1 M phosphate buffer, pH 6.4, were added to 990 μl of spore solution. The effects of the esiRNAs/ERNAs on the growth of B. cinerea were tested in vitro on potato dextrose agar (PDA) plates. 10 μl from a spore solution having a final concentration of 1×10⁵/ml were added to the center of a plate. For the treatment, 400-8000 ng of RNA were added to the spore solution, and were then continuously added every 12 h. In addition to the B. cinerea-specific esiRNAs/ERNAs and edsRNAs, siR gf698 (siR GFP) and water were added to the spores as controls. The growth dynamics under the influence of the RNAs compared to the controls were assessed by measuring the fungal colony diameter (ImageJ software) on day 1, 2, 3, and 5 after inoculation.
In vivo/in planta fungal growth experiments. Here, the ability of the esiRNAs/ERNAs to suppress the complete life cycle of B. cinerea in planta was tested. To this end, RNAs at a final concentration of 400-8000 ng were added to the solution of induced spores (1×10⁵/ml, see above) and the solution was incubated for one and a half hours at room temperature. For infection, the leaves of A. thaliana were moistened with a 10 μl drop of the incubation solution. The plants were then stored in high humidity chambers for two days; thereafter, the sizes of the lesions caused by the fungus were compared with those of control plants (ImageJ software) infected with spores containing the siR gf698 control or water (see also FIG. 19). To further determine fungal growth, the tissue of the leaf sections was removed and B. cinerea DNA was quantified by means of qPCR using the standard curve method. The B. cinerea Actin gene and a control gene from a plasmid preparation which had been spiked in the plant samples were also detected. The plasmid control gene was used to estimate the efficiency of the DNA extraction process and to normalize the amplification of cut A. Finally, the quantity of B. cinerea DNA was determined by interpolating the normalized cut A values on the standard curve.
By transcript analysis. In order to evaluate the silencing of the target RNAs in B. cinerea, an mRNA expression analysis was carried out by means of quantitative real-time PCR (qRT-PCR). To this end, 1×10⁵ spores were taken up in 2 mL of liquid medium and RNA was added (see above). The samples were then grown at room temperature with shaking for 24, 48 and 72 hours. RNA was extracted from the fungal sample using TRIzol, according to the manufacturer's instructions. The qRT-PCR was carried out as described in application example 3 (see also FIG. 19).

The esiRNAs/ERNAs listed in table 8 had a significant silencing effect in vitro (FIG. 19) or fungicidal effect (FIG. 19). As is also clear from FIG. 19, the treatment in planta, even with nonspecific NAs, had a slightly fungicidal effect; however, the treatment with eNAs specifically directed against target RNAs had a significantly stronger fungicidal effect.

Summary of application example 4: the stated problems were solved. According to the invention, esiRNA/ERNA active agents or eNA active agents derived therefrom were identified which have a nematicidal effect against B. cinerea and can be used in plant protection against B. cinerea. The identified eNAs can be used either in unmodified form or in a form modified at 1 to 7 nucleotides for the purpose of protection against all known B. cinerea variants. The eNAs can either be used in plant protection individually or, much more effectively, as a combination (mix) in RNA silencing approaches against B. cinerea. It was also possible to use edsRNAs containing the sequences of identified esiRNAs/ERNAs or other sRNAs derived therefrom in plant protection against B. cinerea.

### REFERENCES

1. Shabalina, S. A. and Koonin, E. V. (2008) Origins and evolution of eukaryotic RNA interference, Trends Ecol Evol, 23, 578-587.
2. Carthew, R. W. and Sontheimer, E. J. (2009) Origins and Mechanisms of miRNAs and siRNAs, Cell, 136, 642-655.
3. Borges, F. and Martienssen, R. A. (2015) The expanding world of small RNAs in plants, Nat Rev Mol Cell Biol, 16, 727-741.
4. Zhan, J. and Meyers B. C. (2023) Plant small RNAs: Their biogenesis, regulatory roles, and functions, Annu Rev Plant Biol, 74, 21-51.
5. Ding, S.-W. (2010)RNA-based antiviral immunity, Nat Rev Immunol, 10, 632-644.
6. Zvereva, A. S. and Pooggin, M. M. (2012) Silencing and innate immunity in plant defense against viral and non-viral pathogens, Viruses, 4, 2578-2597.
7. Gammon, D. B. and Mello, C. C. (2015)RNA interference-mediated antiviral defense in insects, Curr Opin Insect Sci, 8, 111-120.
8. Guo, Z., Li, Y. and Ding, S.-W. (2019) Small RNA-based antimicrobial immunity, Nat Rev Immunol, 19, 31-44.
9. Fukudome, A. and Fukuhara, T. (2017) Plant dicer-like proteins: double-stranded RNA-cleaving enzymes for small RNA biogenesis, J Plant Res, 130, 33-44.
10. Song, M.-S. and Rossi, J. J. (2017) Molecular mechanisms of Dicer: Endonuclease and enzymatic activity, Biochem J, 474, 1603-1618.
11. Deleris, A., Gallego-Bartolome, J., Bao, J., Kasschau, K. D., Carrington, J. C. and Voinnet, O. (2006) Hierarchical action and inhibition of plant Dicer-like proteins in antiviral defense, Science, 313, 68-71.
12. Parent, J.-S., Bouteiller, N., Elmayan, T. and Vaucheret, H. (2015) Respective contributions of Arabidopsis DCL2 and DCL4 to RNA silencing, Plant J, 81, 223-232.
13. Elbashir, S. M., Harborth, J., Lendeckel, W., Yalcin, A., Weber, K. and Tuschl, T. (2001a) Duplexes of 21-nucleotide RNAs mediate RNA interference in cultured mammalian cells, Nature, 411, 494-498.
14. Elbashir, S. M., Martinez, J., Patkaniowska, A., Lendeckel, W. and Tuschl, T. (2001b) Functional anatomy of siRNAs for mediating efficient RNAi in Drosophila melanogaster embryo lysate, EMBO J, 20, 6877-6888.
15. Meister, G. (2013) Argonaute proteins: functional insights and emerging roles, Nat Rev Genet, 14, 447-459.
16. Kobayashi, H. and Tomari, Y. (2016) RISC assembly: Coordination between small RNAs and Argonaute proteins, Biochim Biophys Acta, 1859, 71-81.
17. Carbonell, A. and Carrington, J. C. (2015) Antiviral roles of plant ARGONAUTES, Curr Opin Plant Biol, 27, 111-117.
18. Mi, S., Cai, T., Hu, Y., Chen, Y., Hodges, E., Ni, F., Wu, L., Li, S., Zhou, H., Long, C., Chen, S., Hannon, G. J. and Qi, Y. (2008) Sorting of small RNAs into Arabidopsis argonaute complexes is directed by the 5′ terminal nucleotide, Cell, 133, 116-127.
19. Takeda, A., Iwasaki, S., Watanabe, T., Utsumi, M. and Watanabe, Y. (2008) The mechanism selecting the guide strand from small RNA duplexes is different among argonaute proteins, Plant Cell Physiol, 49, 493-500.
20. Schuck, J., Gursinsky, T., Pantaleo, V., Burgyán, J. and Behrens, S.-E. (2013) AGO/RISC-mediated antiviral RNA silencing in a plant in vitro system, Nucleic Acids Res, 41, 5090-5103.
21. Liu, Q., Wang, F. and Axtell, M. J. (2014) Analysis of complementarity requirements for plant microRNA targeting using a Nicotiana benthamiana quantitative transient assay, Plant Cell, 26, 741-753.
22. Elbashir, S. M., Lendeckel, W. and Tuschl, T. (2001c)RNA interference is mediated by 21- and 22-nucleotide RNAs, Genes Dev, 15, 188-200.
23. Wang, Y., Sheng, G., Juranek, S., Tuschl, T. and Patel, D. J. (2008) Structure of the guide-strand-containing argonaute silencing complex, Nature, 456, 209-213.
24. Brodersen, P., Sakvarelidze-Achard, L., Bruun-Rasmussen, M., Dunoyer, P., Yamamoto, Y. Y., Sieburth, L. and Voinnet, O. (2008) Widespread translational inhibition by plant miRNAs and siRNAs, Science, 320, 1185-1190.
25. Iwakawa, H.-O. and Tomari, Y. (2013) Molecular insights into microRNA-mediated translational repression in plants, Mol Cell, 52, 591-601.
26. Khalid, A., Zhang, Q., Yasir, M. and Li, F. (2017) Small RNA Based Genetic Engineering for Plant Viral Resistance: Application in Crop Protection, Front Microbiol, 8, 43.
27. Pooggin, M. M. (2017)RNAi-mediated resistance to viruses: a critical assessment of methodologies, Curr Opin Virol, 26, 28-35.
28. Tomilov, A. A., Tomilova, N. B., Wroblewski, T., Michelmore, R. and Yoder, J. I. (2008) Trans-specific gene silencing between host and parasitic plants, Plant J, 56, 389-397.
29. Banerjee, S., Banerjee, A., Gill, S. S., Gupta, O. P., Dahuja, A., Jain, P. K. and Sirohi, A. (2017) RNA Interference: A Novel Source of Resistance to Combat Plant Parasitic Nematodes, Front Plant Sci, 8, 834.
30. Majumdar, R., Rajasekaran, K. and Cary, J. W. (2017)RNA Interference (RNAi) as a Potential Tool for Control of Mycotoxin Contamination in Crop Plants: Concepts and Considerations, Front Plant Sci, 8, 200.
31. Price, D. R. G. and Gatehouse, J. A. (2008)RNAi-mediated crop protection against insects, Trends Biotechnol, 26, 393-400.
32. Setten, R. L., Rossi, J. J. and Han, S.-P. (2019) The current state and future directions of RNAi-based therapeutics, Nat Rev Drug Discov, 18, 421-446.
33. Qureshi, A., Tantray, V. G., Kirmani, A. R. and Ahangar, A. G. (2018) A review on current status of antiviral siRNA, Rev Med Virol, 28, e1976.
34. Herrera-Estrella, L., Simpson, J. and Martínez-Trujillo, M. (2005) Transgenic plants: an historical perspective, Methods Mol Biol, 286, 3-32.
35. Dong, O. X. and Ronald, P. C. (2019) Genetic Engineering for Disease Resistance in Plants: Recent Progress and Future Perspectives, Plant Physiol, 180, 26-38.
36. Koch, A. and Wassenegger, M. (2021) Host-induced gene silencing-mechanisms and applications, New Phytol, 231, 54-59.
37. Jan, F.-J., Pang, S.-Z., Tricoli, D. M. and Gonsalves, D. (2000) Evidence that resistance in squash mosaic comovirus coat protein-transgenic plants is affected by plant developmental stage and enhanced by combination of transgenes from different lines, J Gen Virol, 81, 2299-2306.
38. Savenkov, E. I. and Valkonen, J. P. T. (2001) Coat protein gene-mediated resistance to Potato virus A in transgenic plants is suppressed following infection with another potyvirus, J Gen Virol, 82, 2275-2278.
39. Simón-Mateo, C. and García, J. A. (2006) MicroRNA-guided processing impairs Plum pox virus replication, but the virus readily evolves to escape this silencing mechanism, J Virol, 80, 2429-2436.
40. Tabashnik, B. E., Brévault, T. and Carrière, Y. (2013) Insect resistance to Bt crops: lessons from the first billion acres, Nat Biotechnol, 31, 510-521.
41. Kung, Y.-J., You, B.-J., Raja, J. A. J., Chen, K.-C., Huang, C.-H., Bau, H.-J., Yang, C.-F., Huang, C.-H., Chang, C.-P. and Yeh, S.-D. (2015) Nucleotide sequence-homology-independent breakdown of transgenic resistance by more virulent virus strains and a potential solution, Sci Rep, 5, 9804.
42. Lucht, J. M. (2015) Public Acceptance of Plant Biotechnology and GM Crops, Viruses, 7, 4254-4281.
43. Dalakouras, A., Wassenegger, M., Dadami, E., Ganopoulos, I., Pappas, M. L. and Papadopoulou, K. (2020) Genetically Modified Organism-Free RNA Interference: Exogenous Application of RNA Molecules in Plants, Plant Physiol, 182, 38-50.
44. Robinson, K. E., Worrall, E. A. and Mitter, N. (2014) Double stranded RNA expression and its topical application for non-transgenic resistance to plant viruses, J Plant Biochem Biotechnol, 23, 231-237.
45. Kaur, G., Cheung, H.-C., Xu, W., Wong, J. V., Chan, F. F., Li, Y., McReynolds, L. and Huang, L. (2018) Milligram scale production of potent recombinant small interfering RNAs in Escherichia coli, Biotechnol Bioeng, 115, 2280-2291.
46. Le Page, M. (2017) Gene-silencing spray lets us modify plants without changing DNA, New Scientist, 3108.
47. Dowdy, S. F. (2017) Overcoming cellular barriers for RNA therapeutics, Nat Biotechnol, 35, 222-229.
48. Jackson, A. L. and Linsley, P. S. (2004) Noise amidst the silence: off-target effects of siRNAs?, Trends Genet, 20, 521-524.
49. Jackson, A. L., Burchard, J., Schelter, J., Chau, B. N., Cleary, M., Lim, L. and Linsley, P. S. (2006) Widespread siRNA “off-target” transcript silencing mediated by seed region sequence complementarity, RNA, 12, 1179-1187.
50. Senthil-Kumar, M. and Mysore, K. S. (2011) Caveat of RNAi in plants: the off-target effect, Methods Mol Biol, 744, 13-25.
51. Casacuberta, J. M., Devos, Y., Du Jardin, P., Ramon, M., Vaucheret, H. and Nogué, F. (2015) Biotechnological uses of RNAi in plants: risk assessment considerations, Trends Biotechnol, 33, 145-147.
52. Kamola, P. J., Nakano, Y., Takahashi, T., Wilson, P. A. and Ui-Tei, K. (2015) The siRNA Non-seed Region and Its Target Sequences Are Auxiliary Determinants of Off-Target Effects, PLOS Comput Biol, 11, e1004656.
53. Qu, J., Ye, J. and Fang, R. (2012) Artificial microRNAs for plant virus resistance, Methods Mol Biol, 894, 209-222.
54. Dalakouras, A., Wassenegger, M., McMillan, J. N., Cardoza, V., Maegele, I., Dadami, E., Runne, M., Krczal, G. and Wassenegger, M. (2016) Induction of Silencing in Plants by High-Pressure Spraying of In vitro-Synthesized Small RNAs, Front Plant Sci, 7, 1327.
55. Birmingham, A., Anderson, E., Sullivan, K., Reynolds, A., Boese, Q., Leake, D., Karpilow, J. and Khvorova, A. (2007) A protocol for designing siRNAs with high functionality and specificity, Nat Protoc, 2, 2068-2078.
56. Cerritelli, S. M. and Crouch, R. J. (2009) Ribonuclease H: the enzymes in eukaryotes, FEBS J, 276, 1494-1505.
57. Miozzi, L., Gambino, G., Burgyan, J. and Pantaleo, V. (2013) Genome-wide identification of viral and host transcripts targeted by viral siRNAs in Vitis vinifera, Mol Plant Pathol, 14, 30-43.
58. Fakhr, E., Zare, F. and Teimoori-Toolabi, L. (2016) Precise and efficient siRNA design: a key point in competent gene silencing, Cancer Gene Ther, 23, 73-82.
59. Carbonell, A., López, C. and Daròs, J.-A. (2018) Fast-forward Identification of Highly Effective Artificial Small RNAs Against Different Tomato spotted wilt virus Isolates, Mol Plant Microbe Interact, 32, 142-156.
60. Eastman, P., Shi, J., Ramsundar, B. and Pande, V. S. (2018) Solving the RNA design problem with reinforcement learning, PLOS Comput Biol, 14, e1006176.
61. Han, Y., He, F., Chen, Y., Liu, Y. and Yu, H. (2018) SiRNA silencing efficacy prediction based on a deep architecture, BMC Genomics, 19, 669.
62. Gago-Zachert, S., Schuck, J., Weinholdt, C., Knoblich, M., Große, I., Pantaleo, V., Gursinsky, T. and Behrens, S.-E. (2019) Highly efficacious antiviral protection of plants by small interfering RNAs identified in vitro, Nucleic Acids Res, 47, 9343-9357.
63. Shen, X. and Corey, D. R. (2018) Chemistry, mechanism and clinical status of antisense oligonucleotides and duplex RNAs, Nucleic Acids Res, 46, 1584-1600.
64. Bennett, C. F. (2019) Therapeutic Antisense Oligonucleotides Are Coming of Age, Annu Rev Med, 70, 307-321
65. Wdowikowska, A. and Janicka, M. (2022) Antisense oligonucleotide technology as a research tool in plant biology, Funct Plant Biol, 49, 1-12.
66. Crooke, S. T., Liang, X-H, Bakre B. F., Crooke, R. M. (2021) Antisense technology: A review, J Biol Chem 296, 100416
67. Bachman, P., Fischer, J., Song, Z., Urbanczyk-Wochniak, E. and Watson, G. (2020) Environmental Fate and Dissipation of Applied dsRNA in Soil, Aquatic Systems, and Plants, Front Plant Sci, 11, 21.
68. Scholthof, K.-B. G., Adkins, S., Czosnek, H., Palukaitis, P., Jacquot, E., Hohn, T., Hohn, B., Saunders, K., Candresse, T., Ahlquist, P., Hemenway, C. and Foster, G. D. (2011) Top 10 plant viruses in molecular plant pathology, Mol Plant Pathol, 12, 938-954.
69. Rybicki, E. P. (2015) A Top Ten list for economically important plant viruses, Arch Virol, 160, 17-20.
70. Gallitelli, D. (2000) The ecology of Cucumber mosaic virus and sustainable agriculture, Virus Res, 71, 9-21.
71. Garcia-Arenal, F. and Palukaitis, P. (2008) Cucumber Mosaic Virus, In: Mahy, B. W. J. and van Regenmortel, M. H. V. (eds.), Encyclopedia of Virology, 3^{rd} Ed., Academic Press, Oxford, 614-619.
72. Jacquemond, M. (2012) Cucumber mosaic virus, Adv Virus Res, 84, 439-504.
73. Roossinck, M. J. (2001) Cucumber mosaic virus, a model for RNA virus evolution, Mol Plant Pathol, 2, 59-63.
74. Roossinck, M. J. (2002) Evolutionary history of Cucumber mosaic virus deduced by phylogenetic analyses, J Virol, 76, 3382-3387.
75. Palukaitis, P. (2016) Satellite RNAs and Satellite Viruses, Mol Plant Microbe Interact, 29, 181-186.
76. Nouri, S., Arevalo R., Falk B. W. and Russell, L. G. (2014) Genetic Structure and Molecular Variability of Cucumber mosaic virus Isolates in the United States, PLOS One, 9, e96582.
77. Mochizuki, T. and Ohki, S. T. (2012) Cucumber mosaic virus: viral genes as virulence determinants, Mol Plant Pathol, 13, 217-225.
78. Holeva, M. C., Sklavounos, A., Rajeswaran, R., Pooggin, M. M. and Voloudakis, A. E. (2021) Topical Application of Double-Stranded RNA Targeting 2b and CP Genes of Cucumber mosaic virus Protects Plants against Local and Systemic Viral Infection, Plants (Basel), 10, 963.
79. Ali, A. and Kobayashi, M. (2010) Seed transmission of Cucumber mosaic virus in pepper, J Virol Methods, 163, 234-237.
80. O'Keefe, D. C., Berryman, D. I., Coutts, B. A. and Jones, R. A. C. (2007) Lack of Seed Coat Contamination with Cucumber mosaic virus in Lupin Permits Reliable, Large-Scale Detection of Seed Transmission in Seed Samples, Plant Dis, 91, 504-508.
81. Nicaise, V. (2014) Crop immunity against viruses: outcomes and future challenges, Front Plant Sci, 5, 660.
82. Nematodes, Wikipedia
83. Bird, A. F. and Bird, J. (1991a) The Exoskeleton, In: The structure of nematodes, 2^{nd} Ed., Academic Press Inc, San Diego, 44-74.
84. Perry, R. N. and Moens, M. (2011) Introduction to plant-parasitic nematodes: modes of parasitism, In: Jones, J., Fenoll, C. and Gheysen, G. (eds.), Genomics and Molecular Genetics of Plant-Nematode Interactions, Springer Netherlands, Dordrecht, 3-20.
85. Bird, A. F. and Bird, J. (1991b) The Nervous System, In: The structure of nematodes, 2^{nd} Ed., Academic Press Inc, San Diego, 129-156.
86. Bird, A. F. and Bird, J. (1991c) Digestive System, In: The structure of nematodes, 2^{nd} Ed., Academic Press Inc, San Diego, 183-229.
87. Blok, V. C., Jones, J. T., Phillips, M. S. and Trudgill, D. L. (2008) Parasitism genes and host range disparities in biotrophic nematodes: the conundrum of polyphagy versus specialization, BioEssays, 30, 249-259.
88. Perry, R. N. (1996) Chemoreception in plant parasitic nematodes, Annu Rev Phytopathol, 34, 181-199.
89. Semblat, J. P., Rosso, M. N., Hussey, R. S., Abad, P. and Castagnone-Sereno, P. (2001) Molecular cloning of a cDNA encoding an amphid-secreted putative avirulence protein from the root-knot nematode Meloidogyne incognita, Mol Plant Microbe Interact, 14, 72-79.
90. Curtis, R. H. C. (2007) Plant parasitic nematode proteins and the host-parasite interaction, Brief Funct Genomic Proteomic, 6, 50-58.
91. Trudgill, D. L. and Blok, V. C. (2001) Apomictic, polyphagous root-knot nematodes: exceptionally successful and damaging biotrophic root pathogens, Annu Rev Phytopathol, 39, 53-77.
92. Abad, P. and Williamson, V. M. (2010) Plant nematode interaction: a sophisticated dialogue, Adv Bot Res, 53, 147-192.
93. Jones, M. G. K. and Payne, H. L. (1978) Early stages of nematode-induced giant-cell formation in roots of Impatiens balsamina, J Nematol, 10, 70-84.
94. Caillaud, M.-C., Dubreuil, G., Quentin, M., Perfus-Barbeoch, L., Lecomte, P., de Almeida Engler, J., Abad, P., Rosso, M.-N. and Favery, B. (2008) Root-knot nematodes manipulate plant cell functions during a compatible interaction, J Plant Physiol, 165, 104-113.
95. Castagnone-Sereno, P., Danchin, E. G. J., Perfus-Barbeoch, L. and Abad, P. (2013) Diversity and evolution of root-knot nematodes, genus Meloidogyne: new insights from the genomic era, Annu Rev Phytopathol, 51, 203-220.
96. Papadopoulou, J. and Traintaphyllou, A. C. (1982) Sex differentiation in Meloidogyne incognita and anatomical evidence of sex reversal, J Nematol, 14, 549-566.
97. Hussey, R. S. and Mims, C. W. (1991) Ultrastructure of feeding tubes formed in giant-cells induced in plants by the root-knot nematode Meloidogyne incognita, Protoplasma, 162, 99-107.
98. Chitwood, D. J. and Perry, R. N. (2009) Reproduction, physiology and biochemistry, In: Perry, R. N., Moens, M. and Starr, J. L. (eds.), Root-knot nematodes, CABI, Wallingford, 182-200.
99. Curtis, R. H., Robinson, A. F. and Perry, R. N. (2009). Hatch and host location, In: Perry, R. N., Moens, M. and Starr, J. L. (eds.) Root-knot nematodes, CABI, Wallingford, 139-162.
100. Evans, K., Trudgill, D. L. and Webster, J. M. (eds.) (1993) Plant parasitic nematodes in temperate agriculture, CABI, Wallingford.
101. Chen, S. Y. and Dickson, D. W. (2004) Biological control of nematodes by fungal antagonists, In: Chen, Z. X., Chen, S. Y. and Dickson, D. W. (eds.), Nematology: advances and perspectives. Volume 2: Nematode management and utilization, CABI, Wallingford, 979-1039.
102. Stirling, G. R. (2014) Biological control of plant-parasitic nematodes: soil ecosystem management in sustainable agriculture. CABI, Wallingford.
103. Arguel, M.-J., Jaouannet, M., Magliano, M., Abad, P. and Rosso, M.-N. (2012) siRNAs trigger efficient silencing of a parasitism gene in plant parasitic root-knot nematodes, Genes, 3, 391-408.
104. Bakhetia, M., Charlton, W., Atkinson, H. J. and McPherson, M. J. (2005)RNA interference of dual oxidase in the plant nematode Meloidogyne incognita, Mol Plant Microbe Interact, 18, 1099-1106.
105. Banakar, P., Hada, A., Papolu, P. K. and Rao, U. (2020) Simultaneous RNAi Knockdown of Three FMRFamide-Like Peptide Genes, Mi-flp1, Mi-flp12, and Mi-flp18 Provides Resistance to Root-Knot Nematode, Meloidogyne incognita, Front Microbiol, 11, 573916.
106. Chaudhary, S., Dutta, T. K., Shivakumara, T. N. and Rao, U. (2019)RNAi of esophageal gland-specific gene Mi-msp-1 alters early stage infection behaviour of root-knot nematode, Meloidogyne incognita, J Gen Plant Pathol, 85, 232-242.
107. Dalzell, J. J., McMaster, S., Fleming, C. C. and Maule, A. G. (2010a) Short interfering RNA-mediated gene silencing in Globodera pallida and Meloidogyne incognita infective stage juveniles, Int J Parasitol, 40, 91-100.
108. Dalzell, J. J., Warnock, N. D., Stevenson, M. A., Mousley, A., Fleming, C. C. and Maule, A. G. (2010b). Short interfering RNA-mediated knockdown of drosha and pasha in undifferentiated Meloidogyne incognita eggs leads to irregular growth and embryonic lethality, Int J Parasitol, 40, 1303-1310.
109. Danchin, E. G., Arguel, M. J., Campan-Fournier, A., Perfus-Barbeoch, L., Magliano, M., Rosso, M.-N., Da Rocha, M., Da Silva, C., Nottet, N., Labadie, K., Guy, J., Artiguenave, F. and Abad, P. (2013) Identification of novel target genes for safer and more specific control of root-knot nematodes from a pan-genome mining, PLOS Pathog, 9, e1003745.
110. Dong, L., Li, X., Huang, L., Gao, Y., Zhong, L., Zheng, Y. and Zuo, Y. (2014) Lauric acid in crown daisy root exudate potently regulates root-knot nematode chemotaxis and disrupts Mi-flp-18 expression to block infection, J Exp Bot, 65, 131-141.
111. Dong, L., Xu, J., Chen, S., Li, X. and Zuo, Y. (2016) Mi-flp-18 and Mi-mpk-1 genes are potential targets for Meloidogyne incognita control, J Parasitol, 102, 208-213.
112. Dutta, T. K., Papolu, P. K., Banakar, P., Choudhary, D., Sirohi, A. and Rao, U. (2015) Tomato transgenic plants expressing hairpin construct of a nematode protease gene conferred enhanced resistance to root-knot nematodes, Front Microbiol, 6, 260.
113. Huang, G., Allen, R., Davis, E. L., Baum, T. J. and Hussey, R. S. (2006) Engineering broad root-knot resistance in transgenic plants by RNAi silencing of a conserved and essential root-knot nematode parasitism gene, Proc Natl Acad Sci USA, 103, 14302-14306.
114. Iqbal, S., Fosu-Nyarko, J. and Jones, M. G. K. (2020) Attempt to silence genes of the RNAi pathways of the root-knot nematode, Meloidogyne incognita results in diverse responses including increase and no change in expression of some genes, Front Plant Sci, 11, 328.
115. Niu, J., Jian, H., Xu, J., Chen, C., Guo, Q., Liu, Q. and Guo, Y. (2012)RNAi silencing of the Meloidogyne incognita Rpn7 gene reduces nematode parasitic success, Eur J Plant Pathol, 134, 131-144.
116. Papolu, P. K., Gantasala, N. P., Kamaraju, D., Banakar, P., Sreevathsa, R. and Rao, U. (2013) Utility of host delivered RNAi of two FMRF amide like peptides, flp-14 and flp-18, for the management of root knot nematode, Meloidogyne incognita, PLOS One, 8, e80603.
117. Shivakumara, T. N., Papolu, P. K., Dutta, T. K., Kamaraju, D., Chaudhary, S. and Rao, U. (2016) RNAi-induced silencing of an effector confers transcriptional oscillation in another group of effectors in the root-knot nematode, Meloidogyne incognita, Nematology, 18, 857-870.
118. Elad, Y. (1997) Responses of plants to infection by Botrytis cinerea and novel means involved in reducing their susceptibility to infection, Biol Rev, 72, 381-422.
119. Choquer, M., Fournier, E., Kunz, C., Levis, C., Pradier, J.-M., Simon, A. and Viaud, M. (2007) Botrytis cinerea virulence factors: new insights into a necrotrophic and polyphageous pathogen, FEMS Microbiol Lett, 277, 1-10.
120. van Kan, J. A. L. (2006) Licensed to kill: the lifestyle of a necrotrophic plant pathogen, Trends Plant Sci, 11, 247-253.
121. Williamson, B., Tudzynski, B., Tudzynski, P. and van Kan, J. A. L. (2007) Botrytis cinerea: the cause of grey mould disease, Mol Plant Pathol, 8, 561-580.
122. Nakajima, M. and Akutsu, K. (2014) Virulence factors of Botrytis cinerea, J Gen Plant Pathol, 80, 15-23.
123. Pinedo, C., Wang, C.-M., Pradier, J.-M., Dalmais, B., Choquer, M., Le Pêcheur, P., Morgant, G., Collado, I. G., Cane, D. E. and Viaud, M. (2008) Sesquiterpene synthase from the botrydial biosynthetic gene cluster of the phytopathogen Botrytis cinerea, ACS Chem Biol, 3, 791-801.
124. Dean, R., van Kan, J. A. L., Pretorius, Z. A., Hammond-Kosack, K. E., Di Pietro, A., Spanu, P. D., Rudd, J. J., Dickman, M., Kahmann, R., Ellis, J. and Foster, G. D. (2012) The Top 10 fungal pathogens in molecular plant pathology, Mol Plant Pathol, 13, 414-430.
125. Pedras, M. S. C., Hossain, S. and Snitynsky, R. B. (2011) Detoxification of cruciferous phytoalexins in Botrytis cinerea: Spontaneous dimerization of a camalexin metabolite, Phytochemistry, 72, 199-206.
126. Villa-Rojas, R., Sosa-Morales, M. E., López-Malo, A. and Tang, J. (2012) Thermal inactivation of Botrytis cinerea conidia in synthetic medium and strawberry puree, Int J Food Microbiol, 155, 269-272. 127. Malhat, F. M., Haggag, M. N., Loutfy, N. M., Osman, M. A. M. and Ahmed, M. T. (2015) Residues of organochlorine and synthetic pyrethroid pesticides in honey, an indicator of ambient environment, a pilot study, Chemosphere, 120, 457-461.
128. Oliveira, B. R., Penetra, A., Cardoso, V. V., Benoliel, M. J., Barreto Crespo, M. T., Samson, R. A. and Pereira, V. J. (2015) Biodegradation of pesticides using fungi species found in the aquatic environment, Environ Sci Pollut Res Int, 22, 11781-11791.
129. Tomenson, J. A. and Matthews, G. A. (2009) Causes and types of health effects during the use of crop protection chemicals: data from a survey of over 6,300 smallholder applicators in 24 different countries, Int Arch Occup Environ Health, 82, 935-949.
130. Leroux, P. (2007) Chemical Control of Botrytis and its Resistance to Chemical Fungicides, In: Elad, Y., Williamson, B., Tudzynski, P. and Delen, N. (eds.) Botrytis: Biology, Pathology and Control, Springer Netherlands, Dordrecht, 195-222.
131. Garfinkel, A. R. (2021) The history of Botrytis taxonomy, the rise of phylogenetics, and implications for species recognition, Phytopathology, 111, 437-454.
132. Fournier, E., Levis, C., Fortini, D., Leroux, P., Giraud, T. and Brygoo, Y. (2003) Characterization of Bc-hch, the Botrytis cinerea homolog of the Neurospora crassa het-c vegetative incompatibility locus, and its use as a population marker, Mycologia, 95, 251-261.
133. Fournier, E., Giraud, T., Albertini, C. and Brygoo, Y. (2005) Partition of the Botrytis cinerea complex in France using multiple gene genealogies, Mycologia, 97, 1251-1267.
134. Nair, N. G., Guilbaud-Oulton, S., Barchia, I. and Emmett, R. (1995) Significance of carry over inoculum, flower infection and latency on the incidence of Botrytis cinerea in berries of grapevines at harvest in New South Wales, Aust J Exp Agric, 35, 1177-1180.
135. ten Have, A., Mulder, W., Visser, J. and van Kan, J. A. (1998) The endopolygalacturonase gene Bcpg1 is required for full virulence of Botrytis cinerea, Mol Plant Microbe Interact, 11, 1009-1016.
136. Li, H., Tian, S. and Qin, G. (2019) NADPH oxidase is crucial for the cellular redox homeostasis in fungal pathogen Botrytis cinerea, Mol Plant Microbe Interact, 32, 1508-1516.
137. Liu, J.-K., Chang, H.-W., Liu, Y., Qin, Y. H., Ding, Y.-H., Wang, L., Zhao, Y., Zhang, M.-Z., Cao, S.-N., Li, L.-T., Liu, W., Li, G.-H. and Qin, Q.-M. (2018) The key gluconeogenic gene PCK1 is crucial for virulence of Botrytis cinerea via initiating its conidial germination and host penetration, Environ Microbiol, 20, 1794-1814.
138. Nerva, L., Sandrini, M., Gambino, G. and Chitarra, W. (2020) Double-stranded RNAs (dsRNAs) as a sustainable tool against gray mold (Botrytis cinerea) in grapevine: Effectiveness of different application methods in an open-air environment, Biomolecules, 10, 200.
139. Qiao, L., Lan, C., Capriotti, L., Ah-Fong, A., Nino Sanchez, J., Hamby, R., Heller, J., Zhao, H., Glass, N. L., Judelson, H. S., Mezzetti, B., Niu, D. and Jin, H. (2021) Spray-induced gene silencing for disease control is dependent on the efficiency of pathogen RNA uptake, Plant Biotechnol J, 19, 1756-1768. 140. Schumacher, J., Viaud, M., Simon, A. and Tudzynski, B. (2008) The Ga subunit BCG1, the phospholipase C (BcPLC1) and the calcineurin phosphatase co-ordinately regulate gene expression in the grey mould fungus Botrytis cinerea, Mol Microbiol, 67, 1027-1050.
141. Segmüller, N., Kokkelink, L., Giesbert, S., Odinius, D., van Kan, J. and Tudzynski, P. (2008) NADPH oxidases are involved in differentiation and pathogenicity in Botrytis cinerea, Mol Plant Microbe Interact, 21, 808-819.
142. Soulié, M. C., Perino, C., Piffeteau, A., Choquer, M., Malfatti, P., Cimerman, A., Kunz, C., Boccara, M. and Vidal-Cros, A. (2006) Botrytis cinerea virulence is drastically reduced after disruption of chitin synthase class III gene (Bcchs3a), Cell Microbiol, 8, 1310-1321.
143. Ren, W., Sang, C., Shi, D., Song, X., Zhou, M. and Chen, C. (2018) Ubiquitin-like activating enzymes BcAtg3 and BcAtg7 participate in development and pathogenesis of Botrytis cinerea, Curr Genet, 64, 919-930.
144. Yang, Q., Chen, Y. and Ma, Z. (2013) Involvement of BcVeA and BcVelB in regulating conidiation, pigmentation and virulence in Botrytis cinerea, Fungal Genet Biol, 50, 63-71.
145. Zheng, L., Campbell, M., Murphy, J., Lam, S. and Xu, J. R. (2000) The BMP1 gene is essential for pathogenicity in the gray mold fungus Botrytis cinerea, Mol Plant Microbe Interact, 13, 724-732.
146. Wang, M., Weiberg, A., Lin, F.-M., Thomma, B. P. H. J., Huang, H.-D. and Jin, H. (2016) Bidirectional cross-kingdom RNAi and fungal uptake of external RNAs confer plant protection, Nat Plants, 2, 16151.147. Weiberg, A., Wang, M., Lin, F.-M., Zhao, H., Zhang, Z., Kaloshian, I., Huang, H.-D. and Jin, H. (2013) Fungal small RNAs suppress plant immunity by hijacking host RNA interference pathways, Science, 342, 118-123.
148. McLoughlin, A. G., Wytinck, N., Walker, P. L., Girard, I. J., Rashid, K. Y., de Kievit, T., Fernando, W. G. D., Whyard, S. and Belmonte, M. F. (2018) Identification and application of exogenous dsRNA confers plant protection against Sclerotinia sclerotiorum and Botrytis cinerea, Sci Rep, 8, 7320.
149. Gebremichael, D. E., Haile, Z. M., Negrini, F., Sabbadini, S., Capriotti, L., Mezzetti, B. and Baraldi, E. (2021)RNA interference strategies for future management of plant pathogenic fungi: prospects and challenges, Plants (Basel), 10, 650.
150. Iki, T., Yoshikawa, M., Nishikiori, M., Jaudal, M. C., Matsumoto-Yokoyama, E., Mitsuhara, I., Meshi, T. and Ishikawa, M. (2010) In vitro assembly of plant RNA-induced silencing complexes facilitated by molecular chaperone HSP90. Mol. Cell, 39, 282-291.
151. Gursinsky, T., Schulz, B. and Behrens, S. E. (2009) Replication of Tomato bushy stunt virus RNA in a plant in vitro system. Virology, 390, 250-260.
152. Bradford assay, Wikipedia
153. Firefly luciferase, Wikipedia
154. Roossinck, M. J., Zhang, L. and Hellwald, K. H. (1999) Rearrangements in the 5′ nontranslated region and phylogenetic analyses of cucumber mosaic virus RNA 3 indicate radial evolution of three subgroups, J Virol, 73, 6752-6758.
155. Gursinsky, T., Pirovano, W., Gambino, G., Friedrich, S., Behrens, S.-E. and Pantaleo, V. (2015) Homeologs of the Nicotiana benthamiana antiviral ARGONAUTE1 show different susceptibilities to microRNA168-Mediated Control, Plant Physiol, 168, 938-952.
156. Meyer M. and Masquida B. (2014) cis-acting 5′ hammerhead ribozyme optimization for in vitro transcription of highly structured RNAs, Methods Mol Biol, 1086, 21-40.
157. Avis J. M., Conn G. L. and Walker S. C. (2012). Cis-acting ribozymes for the production of RNA in vitro transcripts with defined 5′ and 3′ ends, Methods Mol Biol, 941, 83-98.
158. Rizzo, T. M. and Palukaitis, P. (1990) Construction of full-length cDNA clones of cucumber mosaic virus RNAs 1, 2 and 3: Generation of infectious RNA transcripts, Mol Gen Genet, 222, 249-256.
159. Jackson A. L. and Linsley, P. S. (2010) Recognizing and avoiding siRNA off-target effects for target identification and therapeutic application, Nat Rev Drug Discov, 9, 57-67.

1. Nucleic acid for the protection of plants against the plant pathogens cucumber mosaic virus, Meloidogyne incognita and Botrytis cinerea, wherein
a. the nucleic acid is a small interfering RNA (siRNA) that consists of 21, 22, 23 or 24 base pairs and contains two single-stranded RNAs selected from a guide strand and a passenger strand, wherein the guide strand and the passenger strand are selected from the group consisting of the nucleic acids having SEQ ID NOs: 1-4, 6-11, 14-17, 21-25, 27-30, 32-37, 40-43, 47-51, 53, 55-65, 69-70, 73, 75-85, 89-90, 93-120, 124, 126-132, 134-138, 140-150, 154, 156-162, 164-168 and 170-180; or
b. the nucleic acid is an siRNA according to group a., wherein at least one of the single-stranded RNAs selected from a guide strand and a passenger strand has modifications at 1 to 7 positions in the nucleotide sequence; or
c. the nucleic acid is a small RNA selected from an siRNA and a micro RNA (miRNA), wherein the RNA double strand thereof consists of complementary or partially complementary nucleic acids of group a. and/or group b.; or
d. the nucleic acid is a double-stranded RNA containing nucleotide sequences of at least two siRNAs or sRNAs of groups a., b., or c.; or
e. the nucleic acid is a single-stranded DNA that consists of 12 to 25 nucleotides and contains a sequence of 12 or more nucleotides which are homologous to one of the nucleotide sequences of the single-stranded RNAs of groups a. or b.; or
f. the nucleic acid is a single-stranded DNA according to group e. which has modifications at 1 to 7 positions in the nucleotide sequence,
wherein the nucleic acid is provided for protection against plant pathogens using a method for the targeted identification of effective small interfering RNAs (esiRNAs/ERNAs) and sRNAs derived therefrom, and also effective antisense DNA oligonucleotides (eASO) of different lengths, collectively referred to as effective Nucleic Acids (eNAs), comprising the steps of
(i) an RNA which was selected as a target for RNA silencing (RNAi) is produced using in vitro transcription and converted to small interfering RNAs (siRNAs) in cytoplasmic extracts of plant cells by means of endogenous Dicer-like proteins (DCL);
(ii) a DCL-generated siRNA pool being formed from the RNA used and the siRNAs contained in this pool being determined by means of RNA-seq analysis;
(iii) a messenger RNA (mRNA) of an argonaute (AGO) protein, which mRNA was synthesized by in vitro transcription, is added to the cytoplasmic plant cell extract, the mRNA being constructed such that it encodes the AGO protein in question with a tag;
(iv) AGO protein molecules are formed using in vitro translation, said AGO protein molecules forming RNA-induced silencing complexes (RISC) with the DCL-generated siRNAs that are present;
(v) tagged siRNA-loaded AGO/RISC are immunoprecipitated from BYL and the bound siRNA guide strands are determined by means of RNA-seq analysis;
(vi) those siRNAs that are enriched in AGO/RISC are determined using the comparison of the RNA-seq data with the RNA-seq data from step (ii);
(vii) subsequently produced synthetically and tested for their functionality in a Slicer assay with labeled target RNA, and
(viii) in this way, esiRNAs/ERNAs are identified, and sRNAs, derived therefrom, as well as eASO, collectively referred to as eNAs, are determined;
wherein
I. in order to form the RISC in step (vii), 0.5 pmol of the mRNA of the AGO protein to be used is translated in a reaction solution containing 50% (v/v) BYL, in the presence of 10-100 nM of the synthetic siRNA to be characterized and a 10-fold excess (0.1-1 μM) of a competitor siRNA (e.g., siR gf698, selected from the group consisting of SEQ ID NOs: 205, 206, 207, and 208);
II. after an incubation time of 2.5 h at 25° C. per batch in step I., 3.4 pmol of a nonspecific mRNA (e.g., encoding the Firefly luciferase protein, SEQ ID NO: 209, Schuck et al., 2013) is added as a further competitor RNA, and also 10 fmol of the target RNA is added, the target RNA being labeled, and the reaction batches are incubated again for 15 min at 25° C.;
III. during the incubation in step II., the target RNA is cleaved by the AGO/RISC formed;
IV. after gel electrophoresis of the extracted RNA, the remaining quantity of target RNA or the resulting cleavage products compared to a control reaction (carried out without siRNA) is quantified by measuring the band intensities (ImageQuantTL or ImageJ);
V. classification as esiRNAs/ERNAs takes place using the cleavage activity (slicer activity) on the target RNA, measured in this way, of each RISC formed with this siRNA,
VI. esiRNAs/ERNAs which endonucleolytically convert at least 25% or more of the quantity of target RNA originally used in method step II. to cleavage products are selected.

2. Nucleic acid according to claim 1, wherein the pathogen is cucumber mosaic virus (CMV) and contains the ribonucleotide or deoxyribonucleotide sequence of at least one nucleic acid which is directed against a target RNA of CMV, wherein the target RNA of CMV is selected from the target RNAs having SEQ ID NOs: 189 and 190.

3. Nucleic acid according to claim 1, wherein the pathogen is cucumber mosaic virus (CMV) and contains the ribonucleotide or deoxyribonucleotide sequence of at least one nucleic acid selected from the group consisting of the nucleic acids having SEQ ID NOs: 1-4, 6-11, 14-17, 21-25, 27-30, 32-37, 40-43, 47-51, 53, 55-66, 69-70, 73, 75-86, 89-90.

4. Nucleic acid according to claim 1, wherein the pathogen is Meloidogyne incognita and contains the ribonucleotide or deoxyribonucleotide sequence of at least one nucleic acid which is directed against a target RNA of Meloidogyne incognita, wherein the target RNA of Meloidogyne incognita is selected from the target RNAs having SEQ ID NOs: 191, 192 and 193.

5. Nucleic acid according to claim 1, wherein the pathogen is Meloidogyne incognita and contains the ribonucleotide or deoxyribonucleotide sequence of at least one nucleic acid selected from the group consisting of the nucleic acids having SEQ ID NOs: 93 to 120.

6. Nucleic acid according to claim 1, wherein the pathogen is Botrytis cinerea and contains the ribonucleotide or deoxyribonucleotide sequence of at least one nucleic acid which is directed against a target RNA of Botrytis cinerea, wherein the target RNA of Botrytis cinerea is selected from the target RNAs having SEQ ID NOs: 194, 195, 196, 197, 198 and 199.

7. Nucleic acid according to claim 1, wherein the pathogen is Botrytis cinerea and contains the ribonucleotide or deoxyribonucleotide sequence of at least one nucleic acid selected from the group consisting of the nucleic acids having SEQ ID NOs: 124, 126-132, 134-138, 140-150, 154, 156-162, 164-168 and 170-180.

8. Nucleic acid according to claim 1, wherein the nucleic acid is a double-stranded RNA and this double-stranded RNA contains nucleotide sequences consisting of pseudo-siRNA sequences and sequences of at least two siRNAs or sRNAs according to group a, b, or c.

9. Nucleic acid according to claim 8, wherein the nucleic acid has blunt or overhanging ends.

10. Nucleic acid according to claim 8, wherein the nucleic acid contains a spacer.

11. Nucleic acid according to claim 10, wherein the pseudo-siRNA sequences and spacer in the nucleic acid contain elements selected from transcription promoters, transcription terminators, transport signals, splice sites and ribozymes.

12. Nucleic acid according to claim 8, wherein the nucleic acid is selected from the group consisting of the nucleic acids having SEQ ID NOs: 181, 182, 185, 186 and 200 to 204.

13. Nucleic acid according to claim 1, the nucleic acid having one or more chemical modifications, wherein the chemical modifications are selected from conjugates, base modifications, sugar modifications, “backbone” modifications, and also sugar-phosphate modifications.

14. Composition for use in pest control in plants, comprising one or more nucleic acids according to claim 1 and one or more carrier substances and/or adjuvants.

15. Use of a nucleic acid or composition according to claim 1 in plants for the prophylaxis and/or treatment of infestations and/or infections by pathogens.
